(19) United States
(12) Patent Application Publication
(10) Pub. No.: US 2017/0051149 A1
(43) Pub. Date: Feb. 23, 2017

(54) AZOARYLS AS REVERSIBLY MODULATABLE TUBULIN INHIBITORS

(71) Applicants: ECOLE NORMALE SUPERIEURE DE LYON, Lyon Cedex 07 (FR); UNIVERSITE CLAUDE BERNARD LYON I, Villeurbanne Cedex (FR); CENTRE NATIONAL DE LA RECHERCHE SCIENTIFIQUE (CNRS), Paris Cedex 16 (FR); LUDWIG-MAXIMILIANS-UNIVERSITÄT MÜNCHEN, Munich (DE)

(72) Inventors: Oliver THORN-SESHOLD, München (DE); Malgorzata BOROWIAK, Brodnica (PL); Dirk TRAUNER, München (DE); Jens HASSERODT, Lyon (FR)

(73) Assignees: ECOLE NORMALE SUPERIEURE DE LYON, Lyon Cedex 07 (FR); UNIVERSITE CLAUDE BERNARD LYON I, Villeurbanne Cedex (FR); CENTRE NATIONAL DE LA RECHERCHE SCIENTIFIQUE (CNRS), Paris Cedex 16 (FR); LUDWIG-MAXIMILIANS-UNIVERSITÄT MÜNCHEN, Munich (DE)

(21) Appl. No.: 15/305,395

(22) PCT Filed: Apr. 29, 2014
(86) PCT No.: PCT/IB2014/001246

§ 371 Date: Oct. 20, 2016

(51) Int. Cl. C09B 29/12; C09B 29/08; C09B 29/036; G01N 33/58; C09B 29/26

(52) U.S. Cl. CPC C09B 29/12; G01N 33/582; C09B 29/26; C09B 29/0048; C09B 29/081

(57) ABSTRACT

The invention concerns a new class of tubulin polymerisation inhibitors and their applications in research and medicine, notably in chemotherapy. The invention proposes new azoaryl derivatives of formula (I):

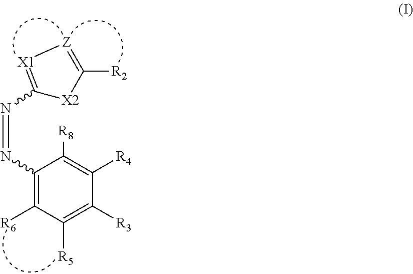

as defined in Claim 1, which may be fully reversibly interconverted between non-tubulin-binding trans and tubulin-binding as isomeric forms, either by irradiation or spontaneously. The invention also concerns compounds with a azoaryl structure for use in studying the cytoskeleton and/or its associated processes, or in the treatment of a disease for which a tubulin polymerisation inhibition activity has a beneficial effect, wherein the compound is administered to the cell, organism or patient in need of such treatment in the trans form of the diazenyl bond, and where this trans form is inactive as regards a tubulin polymerisation inhibition effect, and where after photoisomerisation in vitro, in cellulo or in vivo to an azoaryl compound in its cis isomeric form of the diazenyl bond by the application of light, optionally with modification in vitro, in cellulo or in vivo of one or more substituents, the resultant cis form is active as regards a tubulin polymerisation inhibition effect.

The invention concerns a new class of tubulin polymerisation inhibitors and their applications.

As well as extensive use in research, a notable application of inhibitors of tubulin polymerisation dynamics is as antimitotic chemotherapeutic agents in medicine (eg. Vinca alkaloids, taxanes, etc). All currently clinically used anticancer drugs generate significant toxicity, even though drugs with mechanistic selectivity for some modes of cancer disruption (eg, antivascular agents such as members of the combretastatin family) have been developed. Often the toxicity of chemotherapeutics forces their use at concentrations below the optimal therapeutic dose (dose-limiting toxicity). As a result, while they may show satisfactory in cellulo toxicity, their therapeutic potential in vivo may be limited.

The structurally related colchicine series of antitubulin agents (eg. natural products such as colchicine, podophyllotoxin and steganacin, as well as synthetic analogues including biphenyls, diphenylmethanes, benzopyrans, chalcones, sulfonates, sulfonamides, benzyl phenyl ethers, phenstatins, etc) includes the combretastatin family of stilbenes and stilbene derivatives. Combretastatins (eg. combretastatins A-4 and A-1) are among the most powerfully-acting of the colchicine series, with promising applications including use as both antitubulin and vascular disrupting agents^{[1,2]}. Different prior art works (eg publications^{[1,3,4]} and references therein) have explored in detail their structure-activity relationships (abbreviated SAR), usually evaluated by the biochemical and pharmacological consequences of their antitubulin effects (including tubulin polymerisation inhibition in vitro, cytotoxicity in cellulo, antitumour and antiangiogenic effects in vivo).

This literature shows that members of the combretastatin family display very significantly stronger antitubulin effects in their cis isomeric forms than in their trans isomeric forms^{[2-4]}. One major axis of research into improved combretastatins has therefore focused on developing derivatives more rigidly held in a cis configuration than the original lead compounds, thereby aiming at more powerful biological activity.^{[1,5]}

The group led by Dr. Nobuyuki and Dr. Fukaminato (Research Institute for Electronic Science, Hokkaido University, Japan) works on light-isomerisible azobenzenes which can interact with tubulin and myosin. At the International Symposium on Photochromism in Berlin, 2013, they disclosed an azobenzene compound with the following structure P1:

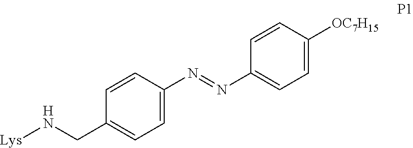

They report that P1 shows light-modulatable cis<->trans isomerism, that the cis and trans isomers show differential inhibition of tubulin polymerization, and that the trans isomer is the more inhibitory form and has an in vitro IC₅₀ (50%-inhibitory concentration for in vitro tubulin polymerisation) of roughly 100 μM. P1 thus displays an IC₅₀ value two orders of magnitude higher than the best of the combretastatin family (eg combretastatin A-4,^{[1]} abbreviated CA4). This may significantly restrict the scope of applications of P1 since at the high doses required for its biological activity, it may encounter problems of solubility, pharmacokinetics, and synthetic cost. It is important in the context of the invention to stress that P1 was reported to be more inhibitory in its trans form than in its cis form. Off-target toxicity is known to be a severely limiting factor for current antitubulin agents;^{[6]} and azobenzenes are well known to spontaneously undergo thermal cis->trans isomerism with first-order kinetics and thus revert completely to their trans form in the absence of illumination^{[7]}: therefore with trans-active azobenzene derivatives such as P1, in order to reduce off-target toxicity, it would be necessary to illuminate near-continuously all the off-targeted zones of the organism to suppress the accumulation of a significant amount of trans isomer, which is impractical for complex settings. Furthermore, even in the irradiated zones, a small but significant percentage of trans isomer almost always remains due to the photostationary state (abbreviated PSS) equilibrium distribution of the compound between cis and trans forms^{[7]}; and that percentage too may generate off-target toxicity in the case of trans-active azobenzenes.

Dr Bisby, Dr Hadfield, Dr McGown and Dr Scherer (University of Salford, UK) have pursued stilbenoid analogues of combretastatins which may be photoisomerised from the trans to the as form. They have explored single-photon absorption for the E (trans) to Z (cis) isomerization of stilbenes such as members of the combretastatin class.^{[8]} This has several practical and theoretical problems, some of which they have discussed themselves^{[9]}, and which are selectively summarised here. The wavelengths required for single-photon stilbene isomerization are typically 310-350 nm, ie well outside the biocompatible wavelength range due to strong tissue scattering and absorption, as well as extreme phototoxicity of such high-energy radiation.^{[10]} The single-photon isomerisation of stilbenes also suffers in general from a low product of absorption coefficient and isomerisation quantum efficiency, which the members of the combretastatin family are not known to overcome. As a result, their activation in vivo to the as form would require a high dose of short-wavelength UV irradiation, incurring light-toxicity problems, as well as light delivery problems (if the light cannot be applied via a very short path length, then scattering and absorptive losses will require that extremely high intensities be used to ensure that enough light reaches the target)^{[10]}.

They have also explored multiphoton absorption to photoswitch combretastatin A-4 from the trans to the as isomer.^{[9]} Although the longer wavelengths needed for multiphoton absorption reduce the problematicity of tissue scatter and biological compatibility of the excitatory light wavelength relative to that for the single-photon process, the intensities required for the non-linear multiphoton absorption process are in general vastly increased relative to single-photon absorption, in the range of MWcm⁻² to GWcm⁻² at the site of desired activation for these compounds^{[9]}. This brings extensive practical problems for light delivery, as the heat to be dissipated during such illumination may itself prove very toxic to a biological sample; it also necessitates elevated cost and complexity in a practical setting, and it still does not address the suboptimal multiphoton absorption cross-section of stilbenoid molecules nor their suboptimal quantum efficiencies of isomerisation.

Yet, regardless of whether single-photon or multiphoton isomerisation is used, a major problem is incurred by invoking the photoisomerisation of stilbenes^{[11,12]}. Cis-stilbenes may efficiently undergo a reversible 6-n electrocyclisation reaction upon absorption of light, giving a metastable dihydrophenanthrene product. This dihydrophenanthrene is however prone to be irreversibly converted to a phenanthrene or other fused polycyclic aromatic system by a variety of spontaneous chemical reactions, eg. oxidation or elimination, especially when molecular oxygen or iron(III) are present (ie. dissolved) in a reaction mixture. This photochemical reaction has been used for decades as an efficient synthetic access to a variety of polycyclic aromatic systems^{[11,12]}. Dissolved oxygen is however a constant feature in medical and biological applications. Thus, exposing a stilbene mixture to light sufficient for photoisomerisation in an in vivo setting may give substantial degradation of the cis-stilbene population by electrocyclisation-oxidation. The planar phenanthrenoid degradation products lack the crucial twisted orientation of the aromatic rings which allows colchicine-site inhibitors of tubulin polymerisation to bind to tubulin, therefore they cannot present antitubulin activity^{[1]}. Consequently, this degradation will deplete the potential biological activity the stilbene could exert in its cis form. There is also the risk that these byproducts act as potent nonspecific toxic carcinogens, as has been well known for planar polycyclic aromatic compounds in general for decades^{[13,14]}. In sophisticated applications where many repeated trans<->cis photoisomerisations are desirable, eg. for reducing off-target effects by dynamic spatiotemporal localisation of each isomeric form, it is considered therefore that stilbenes of the combretastatin class may quickly suffer extensive or total degradation to non-tubulin binding byproducts showing highly undesirable biological activity.

In this context, the purpose of the invention is to propose a new class of tubulin polymerisation inhibitors which, compared to standard “always-active” inhibitors and chemotherapeutics, offers the possibility of reduced undesirable off-target effects by allowing the reversible and spatiotemporal control of their inhibition properties. In this context of the state of the art, the invention proposes a new class of azoaryl derivatives with fully reversibly light-controllable tubulin polymerisation inhibitor activity which are active in a cis isomeric form of the diazenyl bond. These compounds answer the above-mentioned problems facing the prior art, and in particular offer the possibility to reduce undesired off-target inhibitory/toxic effects.

First, the invention concerns the compounds of formula (I):

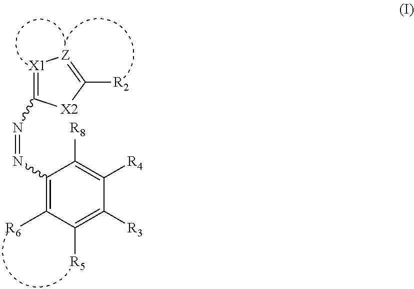

wherein:
the aryl ring containing X2 is denoted the “north ring”, and the aryl ring bearing R₃ is denoted the “south ring”,
dotted lines indicate sites where a fused ring may be present,
X1, X2, Z and R₂ are defined as follows:
   X2 is —C(R₁₀)═C(R₉)—, X1 is C(R₇) and Z is C(R₁); or
   X2 is —C(R₁₀)═C(R₉)—, X1 is C, Z is C, and X1 and Z are linked together to form a fused phenyl ring either unsubstituted or substituted with one or several groups Rₘ, identical or different, such that the north ring is a naphthalene; or
   X2 is —C(R₁₀)═C(R₉)—, X1 is C(R₇), Z is C, and Z and R₂ are linked together forming a fused phenyl ring either unsubstituted or substituted with one or several groups Rₘ, identical or different, such that the north ring is a naphthalene; or
   Z is C(R₁), X2 is S, and X1 is C(R₇); or
   Z is C(R₁), X2 is NH, and X1 is C(R₇); or
   Z is N, X2 is NH, and X1 is C(R₇); or
   Z is C(R₁), X2 is NH, and X1 is N; or
   Z is N, X2 is O, and X1 is C(R₇); or
   Z is C(R₁), X2 is O, and X1 is N; or
   Z is N, X2 is S, and X1 is C(R₇); or
   Z is C(R₁), X2 is S, and X1 is N; or
   X1 is C(R₇), X2 is —C(R₁₀)═C(R₉)—, and Z is N, N(Me)⁺ or N⁺(O⁻); or
   X1 is N, N(Me)⁺ or N⁺(O⁻), Z is C(R₁), and X2 is —C(R₁₀)═C(R₉)—; or
   X1 is N, X2 is —N═C(R₉)—, Z is C(R₁); or
   X1 is C(R₇), X2 is —C(R₁₀)═N—, Z is N; or
   X2 is —C(R₁₀)═C(R₉)—, X1 is C, Z is C, and X1 and Z are linked together and form a fused 2-pyridine ring either unsubstituted or substituted with one or several groups Rₘ, identical or different, such that the north ring is a quinoline; or
   X2 is —C(R₁₀)═C(R₉)—, X1 is C(R₇), Z is C, and R₂ and Z are linked together forming a fused 2-pyridine ring either unsubstituted or substituted with one or several groups Rₘ, identical or different, such that the north ring is a quinoline; or
   X2 is —C(R₁₀)═N—, X1 is CR₇, Z is C, and R₂ and Z are linked together forming a fused phenyl ring unsubstituted or substituted with one or several groups Rₘ, identical or different, such that the north ring is an isoquinoline; or
   X2 is —C(R₁₀)═N—, X1 is C, Z is C, and X1 and Z are linked together forming a fused phenyl ring unsubstituted or substituted with one or several groups Rₘ, identical or different, such that the north ring is an isoquinoline; or
   X2 is —C(R₁₀)═C(R₉)—, X1 is C, Z is C and X1 and Z are linked together and form a fused 3-pyridine ring either unsubstituted or substituted with one or several groups Rₘ, identical or different, such that the north ring is an isoquinoline; or
   X2 is —C(R₁₀)═C(R₉)—, X1 is C(R₇), Z is C, and R₂ and Z are linked together forming a fused 3-pyridine ring either unsubstituted or substituted with one or several groups Rₘ, identical or different, such that the north ring is an isoquinoline; or
   X2 is —N═C(R₉)—, X1 is C(R₇), Z is C and R₂ and Z are linked together forming a fused phenyl ring either unsubstituted or substituted with one or several groups Rₘ, identical or different, such that the north ring is an isoquinoline; or
   X2 is —N═C(R₉)—, X1 is C, Z is C, and X1 and Z are linked together forming a fused phenyl ring either unsubstituted or substituted with one or several groups Rₘ, identical or different, such that the north ring is an isoquinoline; or
   X2 is —C(R₁₀)═C(R₉)—, X1 is C(R₇), Z is C, and R₂ is a group —OCH₂O— or —OCH₂CH₂O— which forms a bridge between Z and the phenyl carbon to which R₂ is joined (in para to the diazenyl bond);
   and R₂, when it is not linked to Z, is chosen among —OCH₃, —OCF₃, —F, —CH₃, —CF₃, —CH₂CH₃, —OCH₂CH₃, —SCH₃, —SCF₃, —NHCH₃, —N(CH₃)₂ and —CN;
R₁ is chosen among hydrogen, —Y₁Rₐ, —S₂R_{b}, —NHR_{d}, —ORₑ, —OPO₃H₂, —NO₂, —B(OH)₂, —B(OR_{b})₂, —B(OR_{b}O), —N₃, —F, —Cl, —Br, —I, —CHO, —CO₂H, —CONH₂, —CN, —NC, —SO₃H, —CO₂R_{b}, —SO₂NH₂ and —R_{b};
R₆ and R₇, identical or different, are chosen among hydrogen, —Y₂R_{f}, —S₂R_{g}, —NHRᵢ, —ORⱼ, —OPO₃H₂, —NO₂, —B(OH)₂, —B(OR_{g})₂, —B(OR_{g}O), —N₃, —F, —Cl, —Br, —I, —CHO, —CO₂H, —CONH₂, —CN, —NC, —SO₃H, —CO₂R_{g}, —SO₂NH₂, —R_{g}, —CO₂NHR_{g}, —CO₂NR_{g}Rₕ, —N-piperazinyl, —N-morpholinyl, piperidinyl, and -linker-reporter units; or R₆ and R₅ are linked together forming a fused phenyl, 2-pyridinyl or 3-pyridinyl ring, the said phenyl, 2-pyridinyl or 3-pyridinyl being unsubstituted or substituted with one or several groups Rₙ, identical or different, such that the south ring is respectively a naphthalene, quinoline or isoquinoline;
R₃ is chosen among —OCH₃, —OCF₃, —F, —CH₃, —CF₃, —CH₂CH₃, —OCH₂CH₃, —SCH₃, —NHCH₃, —N(CH₃)₂ and —CN;
R₄ and R₅, identical or different, are chosen among —OCH₃, —OCF₃, —F, —CH₃, —CF₃, —CH₂CH₃, —OCH₂CH₃, —SCH₃, —SCF₃, —NH₂, —NHCH₃, —N(CH₃)₂ and —CN; or R₅ and R₆ are linked together as outlined above (ie. forming a fused phenyl, 2-pyridinyl or 3-pyridinyl ring, the said phenyl, 2-pyridinyl or 3-pyridinyl being unsubstituted or substituted with one or several groups Rₙ, identical or different, such that the south ring is respectively a naphthalene, quinoline or isoquinoline);
when R₉ is present, it is in meta to the diazenyl bond,
when R₁₀ is present, it is in ortho to the diazenyl bond,
R₃, R₉ and R₁₀, identical or different, are chosen among H, F, Br, Cl or I;
Y₁═O, S, NH or NRₖ;
Y₂═O, S, NH or NRₗ;
Rₐ is chosen among hydrogen, —R_{b}, —COR_{b}, —CO₂R_{b}, —CONH₂, —CONR_{b}R_{c}, —CONHR_{b}, —CH₂OC(O)R_{b}, and cleavable groups, which after cleavage, for instance in vivo, lead either to R₁═—OH when Y₁═O, or to R₁═—NH₂ when Y₁═NH, or to R₁═—NHRₖ when Y₁═NRₖ, or to R₁═—SH when Y₁═S;
R_{b}, R_{c}, R_{g}, Rₕ, Rₖ and Rₗ, identical or different, are chosen among (C₁-C₆)alkyl, (C₁-C₆)alkyl-OH, (C₁-C₆)alkenyl, (C₁-C₆)alkynyl, (C₃-C₇)cycloalkyl, aryl, heteroaryl, heterocycle, (C₁-C₆)alkyl(C₃-C₇)cycloalkyl, (C₁-C₆)alkylaryl, (C₁-C₆)alkylheteroaryl, and (C₁-C₆)alkylheterocycle;
R_{d} and Rᵢ are identical or different, and are a peptidic group attached via its carboxyl terminus;
Rₑ and Rⱼ are identical or different, and are a glycosidyl group;
R_{f} is chosen among hydrogen, —R_{g}, —COR_{g}, —CO₂R_{g}, —CONH₂, —CONR_{g}Rₕ, —CONHR_{g}, —OCH₂OC(O)R_{g}, and cleavable groups which after cleavage, for instance in vivo, lead either to R₆ or R₇═—OH when Y₂═O, to R₆ or R₇═—NH₂ when Y₂═NH, or to R₆ or R₇═—NHRₗ when Y₂═NRₗ, or to R₆ or R₇═—SH when Y₂═S;
Rₘ and Rₙ are identical or different, and are chosen among —CH₃, —OH, —NH₂, —NHCOCH₃, —SO₃H, —CO₂H, —CONH₂, —CO₂CH₃, —PO₃H₂, —NO₂, —B(OH)₂, —N₃, —CN, —C≡CH, and —SO₂NH₂;
   and their hydrates, pharmaceutically acceptable salts and solvates, as a mixture of isomers in any proportions and also as pure isomer;

but explicitly excluding 1-(4-methoxynaphthalen-1-yl)-2-(3,4,5-trimethoxyphenyl)diazene and 1-(4-methoxyphenyl)-2-(3,4,5-trimethoxyphenyl)diazene in all mixtures of isomers and also as pure isomers. The excluded structures correspond to the following compounds P2 and P3 described in 1949-1952 by Seligman et al.^{[13,14]}:

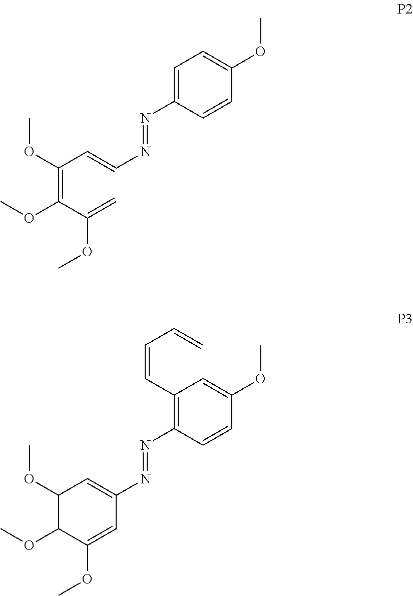

These two compounds were pursued as part of a larger study into polyaromatic carcinogens, which did not consider any effects of these compounds on tubulin. The authors did not attempt any trans<->cis photoisomerisation of either compound; also they found no significant biological activity of P3, and inhibition of only one of the two types of tumour tested with P2; and the biochemical target of the compounds was not determined. Seligman et al. did not consider, nor suggest, the potential for these or similar structures to act as photoswitchable inhibitors of tubulin polymerisation and thus spatiotemporally-controllable photopharmaceuticals, contrary to the inventors of the present invention.

Compounds according to the invention can be used in the transform:

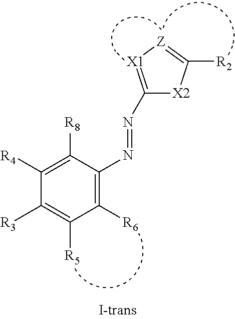

or in the cis form:

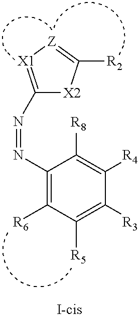

wherein X1, X2, Z, R₂, R₃, R₄, R₅, R₆ and R₈ are as defined for formula (I).

According to preferred embodiments, in compounds of formula (I), X1, X2, Z and R₂ are defined as follows:
X2=—C(R₁₀)═C(R₉)—, X1=C(R₇) and Z═C(R₁); or
X2=—C(R₁₀)═C(R₉)—, X1=C, Z═C, and X1 and Z are linked together to form a fused phenyl ring either unsubstituted or substituted with one or several groups Rₘ, identical or different, such that the north ring is a naphthalene; or
X2=—C(R₁₀)═C(R₉)—, X1=C(R₇), Z═C, and R₂ and Z are linked together forming a fused phenyl ring either unsubstituted or substituted with one or several groups Rₘ, identical or different, such that the north ring is a naphthalene; or
X2=S, X1=C(R₇), and Z═C(R₁); or
X1=C(R₇), X2=—C(R₁₀)═C(R₉)—, and Z═N, N(Me)⁺ or N⁺(O⁻); or
X1=N, N(Me)⁺ or N⁺(O⁻), Z═C(R₁), and X2=—C(R₁₀)═C(R₉)—; or
X1=N, X2=—N═C(R₉)—, Z═C(R₁); or
X2 is —C(R₁₀)═C(R₉)—, X1 is C, Z is C, and X1 and Z are linked together and form a fused 2-pyridine ring either unsubstituted or substituted with one or several groups Rₘ, identical or different, such that the north ring is a quinoline; or
X2 is —C(R₁₀)═C(R₉)—, X1 is C(R₇), Z is C, and R₂ and Z are linked together forming a fused 2-pyridine ring either unsubstituted or substituted with one or several groups Rₘ, identical or different, such that the north ring is a quinoline; or
X2=—C(R₁₀)═C(R₉)—, X1=C(R₇), Z═C and R₂ is a group —OCH₂O— or —OCH₂CH₂O— which forms a bridge between Z and the phenyl carbon to which R₂ is joined (in para to the diazenyl bond);
and R₂, when it is not linked to Z, is chosen among —OCH₃, —OCF₃, —F, —CH₃, —CF₃, —CH₂CH₃, —OCH₂CH₃, —SCH₃, —SCF₃, —NHCH₃, —N(CH₃)₂ and —CN;
   and R₁, R₃, R₄, R₅, R₆, R₇, R₈, R₉, R₁₀, Rₘ and Rₙ are as defined for formula (I).

According to one set of embodiments of the compounds (I), groups X1, X2 and Z are chosen to give (heteroaryldiazenyl)phenyl molecules which may possess improved isomerisation parameters relative to azoaryls of formula (A) while still retaining appropriate steric parameters for satisfactory tubulin binding. Mention may be made of preferred embodiments such as five-membered heteroaryl rings (which preferentially feature R₂═—CH₃) including thiophenes (eg. X2=S, X1=C(R₇), and Z═C(R₁)), imidazoles (eg. Z═N, X2=NH, and X1=C(R₇)), and oxazoles (eg. Z═N, X2=O, and X1=C(R₇)), where in all cases R₇ and R₁ are as defined according to the invention. Mention may also be made of preferred embodiments such as six-membered heteroaryl rings (which preferentially feature R₂═—OCH₃) including pyridines or their simple derivatives (eg. X1=C(R₇), X2=—C(R₁₀)═C(R₉)—, and Z═N or N(Me)⁺ or N⁺O⁻; or else eg. X1=N or N(Me)⁺ or N⁺O⁻, Z═C(R₁), and X2=—C(R₁₀)═C(R₉)—), and pyrimidines (eg. X1=N, X2=—N═C(R₉)—, Z═C(R₁)), where in all cases R₇, R₉, R₁₀ and R₁ are as defined according to the invention.

A preferred family of compounds according to the invention concerns compounds wherein X2 is —C(R₁₀)═C(R₉)—, and corresponding to one of the following formulae:

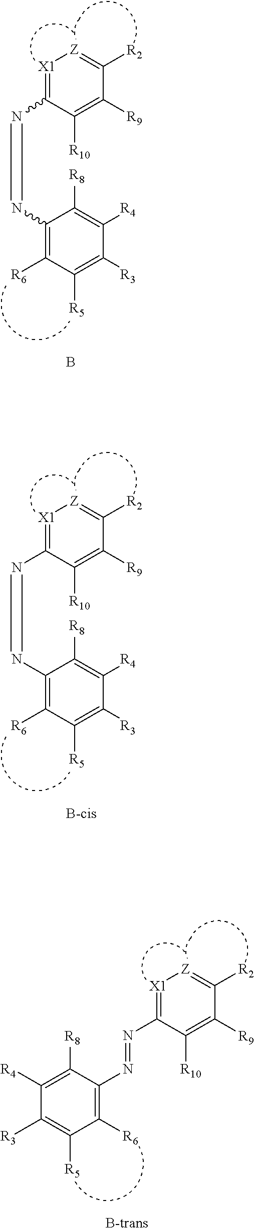

wherein X1, Z and R₂ are defined as follows:
   X1=C(R₇) and Z═C(R₁); or
   X1=C, Z═C, and X1 and Z are linked together to form a fused phenyl ring either unsubstituted or substituted with one or several groups Rₘ, identical or different, such that the north ring is a naphthalene; or
   X1=C(R₇), Z═C, and R₂ and Z are linked together to form a fused phenyl ring either unsubstituted or substituted with one or several groups Rₘ, identical or different, such that the north ring is a naphthalene; or
   X1=C, Z═C and X1 and Z are linked together and form a fused 2-pyridine ring either unsubstituted or substituted with one or several groups Rₘ, identical or different, such that the north ring is a quinoline; or
   X1=C(R₇), Z═C and R₂ and Z are linked together and form a fused 2-pyridine ring either unsubstituted or substituted with one or several groups Rₘ, identical or different, such that the north ring is a quinoline; or
   X1=C(R₇), Z═C and R₂ is a group —OCH₂O— or —OCH₂CH₂O-which forms a bridge between Z and the phenyl carbon to which R₂ is joined (in para to the diazenyl bond)
   and R₂, when it is not linked to Z, is chosen among —OCH₃, —OCF₃, —F, —CH₃, —CF₃, —CH₂CH₃, —OCH₂CH₃, —SCH₃, —SCF₃, —NHCH₃, —N(CH₃)₂ and —CN;
      and R₁, R₃, R₄, R₅, R₆, R₇, R₈, R₉ and R₁₀ are as defined for formula (I).

A particularly preferred family of compounds according to the invention concerns compounds wherein X2 is —C(R₁₀)═C(R₉)—, and corresponding to one of the following formulae:

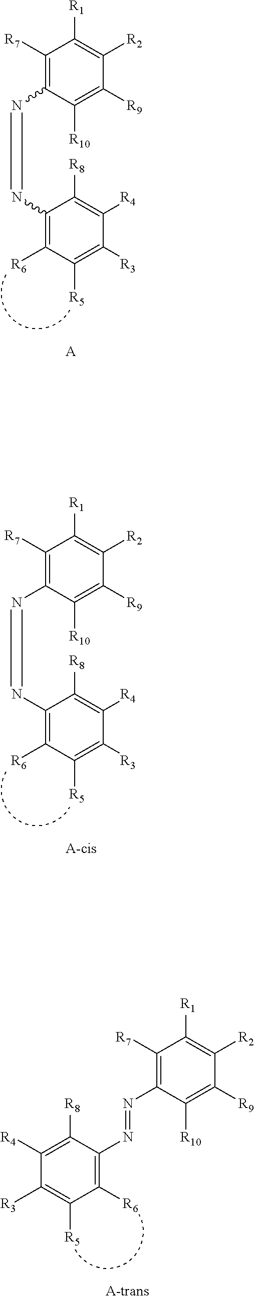

R₁, R₂, R₃, R₄, R₅, R₆, R₇, R₈, R₉ and R₁₀ are as defined for formula (I). Advantageously, at least two of the substituents R₇, R₁, R₂, R₉ and R₁₀ are different from hydrogen and/or at least one of the substituents R₆, R₇ and R₁ are different from hydrogen.

Before detailing the preferred compounds according to the invention, their uses and advantages, a certain number of definitions, conventions and abbreviations are quoted.

When azobenzene compounds of the invention are drawn, they are typically oriented vertically along the N═N bond, with the ring bearing group R₃ drawn at the bottom; this is herein named the “south ring” and corresponds to the generally accepted definition of the “A ring” in the literature of the combretastatins. The other aromatic moiety is called the “north ring” and corresponds to the “B ring” in the literature of the combretastatins.

The cis and trans descriptors which are used in the present invention, are always used to specify the configuration of the diazenyl bond present in the compounds of formula (I), (B) or (A). The compounds of the invention are then typically drawn in the as configuration with the more polar south ring substituents (eg. —OCH₃) oriented towards the right, even if the compounds are intended to represent a mixture of cis and trans forms with undefined ratio. The rotamer where the steric bulk of the north ring is oriented as much as possible away from the south ring is typically depicted. This standard representation then defines the right side of the cis form as the binding face which is likely to be most important for interactions with tubulin. This standard representation is used to achieve consistent numbering of substituents for the purposes of logically defining the compounds of the invention and relating them to the literature of the combretastatins. The compounds of the invention are referred to collectively as (I), (B) or (A) which correspond to two of the preferred families, and these should be understood to include both trans and as isomeric forms, in any relative amounts or in an enriched or pure form of one of the trans and cis isomers. In the description of the invention, as or E and trans or Z are used equally.

The term “alkyl” is intended to mean, when not otherwise specified, a linear or branched, saturated hydrocarbon group. The term “(C₁-C₆)alkyl” is intended to mean an alkyl group which comprises from 1 to 6 carbon atoms. By way of examples of such an alkyl group, mention may be made of methyl, ethyl, n-propyl, isopropyl, n-butyl, isobutyl, sec-butyl, t-butyl, n-pentyl and n-hexyl groups.

The term “alkenyl” is intended to mean a linear or branched, unsaturated hydrocarbon group, including at least one double bond. By way of examples of such an alkenyl group, mention may be made of vinyl (—CH═CH₂), allyl (—CH₂CH═CH₂), and 5-hexenyl (—CH₂CH₂CH₂CH₂CH═CH₂) groups.

The term “alkynyl” is intended to mean a linear or branched, unsaturated hydrocarbon group, including at least one triple bond. By way of examples of such an alkynyl group, mention may be made of acetylenyl (—C≡CH) and propargyl (—CH₂C≡CH) groups.

The term “cycloalkyl” is intended to mean a cyclic and saturated hydrocarbon group. By way of examples of such an cycloalkyl group, mention may be made of cyclopentyl, cyclohexyl and cycloheptyl groups.

The term “aryl” is intended to mean an aromatic hydrocarbon with 6 to 14 carbon atoms. Typical aryl groups are benzene, fluorene and naphthalene.

The term “heteroaryl” is intended to mean an aromatic heterocycle having one or more heteroatoms in the ring chosen among oxygen, sulfur, and nitrogen, as well as 1 to 13 carbon atoms. Non-limiting examples of heteroaryl groups include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, tetrazolyl, tetrazinyl, 1,2,3-triazolyl, 1,2,4-triazolyl and the like.

The term “heterocycle” is intended to mean a non-aromatic heterocycle having at least one heteroatom in the ring. Non-limiting examples of suitable heteroatoms which can be included in the aromatic ring include oxygen, sulfur, and nitrogen. A heterocycle group can have 3 to 10 carbon atoms. Non-limiting examples of heterocycle groups include dihydropyridinyl, piperidinyl, tetrahydrothiophenyl, morpholinyl, tetrahydrofuranyl and pyrrolidyl.

The term “(C₁-C₆)alkyl-OH”, “(C₁-C₆)alkyl(C₃-C₇)cycloalkyl”, “(C₁-C₆)alkylaryl”, “(C₁-C₆)alkylheteroaryl”, and “(C₁-C₆)alkylheterocycle” mean respectively a hydroxy, (C₃-C₇)cycloalkyl, aryl, heteroaryl and heterocycle group linked by a bivalent alkyl (also named alkylene) group comprising from 1 to 6 carbon atoms.

A “peptidic group” is intended to mean preferably a linear oligopeptide sequence of 1 to 4 proteinogenic or non-natural α-amino acids, including D-configured peptides (eg. D-alanine). Each amino acid unit may optionally be in a protective form, and the amine terminus may be the free base, the ammonium salt, or a protective form such as the acetamide. SExamples of such peptidic groups are (L or D)-Leu-, (L or D)-Ser-, or D-Ala-Phe-Lys-^{[15]}, employing the standard three-letter abbreviations used by those skilled in the art. A peptidic group may be the substrate of a peptidase (optionally after in vivo modification of the protecting group or groups), preferentially of an exopeptidase. The peptidic group is either specifically hydrolysed by a limited subset of peptidases (“high specificity substrate”) or else rapidly and potentially nonspecifically hydrolysed (“high activation rate”). Preferentially, a high specificity substrate is chosen if it targets a peptidase of biomedical interest such as a disease-associated peptidase, eg. the peptidic group D-Ala-Phe-Lys- which may be specifically hydrolysed by the tumour-associated peptidase plasmin^{[15,16]}. A high activation rate may be attained especially with monopeptides such as Leu- or Ser-, and these peptidic groups may therefore serve as solubilising moieties in a prodrug strategy to efficiently release an azoaryl compound of the invention after peptidolysis.

The term “glycosidyl group” is intended to mean, when not otherwise specified, a naturally occurring C5-C9 monosaccharide group as is understood by those skilled in the art, optionally in a protective form, such that the glycosidyl group may be the substrate of a glycosidase optionally after in vivo modification of the protecting group(s); preferred examples are -1-(β-D-galactopyranose) (galactopyranose is also named galactosyl), -1-(α-D-galactopyranose), -1-(β-D-glucopyranose), -1-(α-D-glucopyranose), -1-(β-D-glucuronic acid), or -3-(N-acetyl-D-glucosamine).

The term “reporter” is intended to mean, when not otherwise specified, a fluorophore, a chromophore, an antenna or a tag moiety. The reporter is chosen either to allow effecting resonant energy transfer to an azoaryl moiety (eg. by the FRET effect), or else to enable the local concentration of a compound (I) to be measured conveniently and/or sensitively, such as by fluorescence or absorbance measurement, or by selective enzymatic reaction or pulldown of the tag moiety. Preferred examples of reporters therefore include moieties with strong single-photon absorption and/or fluorescence^{[17]} such as a fluorescein, rhodamine, coumarin, phenoxazine, acridine, boron-dipyrromethene (BODIPY), dansyl, propidium, nitrobenzofurazan, resorufin, cyanine, Cascade Yellow, Nile Red, carborhodamine, silarhodamine (SiR), DABCYL, black hole quencher (BHQ) moiety or their analogues; or a moiety known to possess strong two-photon absorption such as (E)-4,4′-bis(diethylamino)stilbene; or well-known pulldown tags such as biotin which may be pulled down by streptavidin; or enzymatic reaction tags such as substrate moieties for enzymatic labelling systems such as the “SNAP-tag” (eg. substrate O⁶-benzylguanine), “CLIP-tag” (eg. substrate O²-benzylcytosine) or “Halo-tag” (eg. substrate —((CH₂)₂O)₂(CH₂)₆Cl) systems.

The term “linker” is intended to mean, when not otherwise specified, a low-molecular-weight bifunctional group which is at one end covalently attached to the azoaryl moiety of a compound according to the invention, and at the other end is attached to a reporter. Preferred linkers include but are not limited to (C₁-C₁₂)alkylene (eg L1); (C₁-C₁₂)alkenylene (eg L4); —(CH₂)ₘ₁(C₃-C₇)cycloalkyl(CH₂)ₘ₂— and —(CH₂)ₘ₁aryl(CH₂)ₘ₂— (eg L3), with m1 and m2, identical or different, being integers chosen in the range 0 to 6; or else a moiety including between 1 to 10 carbon atoms and 1 to 6 heteroatoms chosen from among oxygen, nitrogen and sulfur, which therefore includes but is not limited to typical linker systems such as —(CH₂)ₘ₁heteroaryl(CH₂)ₘ₂— especially when heteroaryl is a triazole, tetrazole or pyridazine (eg 15 and L6), —(CH₂)ₘ₁heterocycle(CH₂)ₘ₂—, oligo(ethyleneglycol) (eg L7), —(CH₂)ₘ₁—C(O)O—(CH₂)ₘ₂—, —(CH₂)ₘ₁—C(O)NH—(CH₂)ₘ₂—, —C(O)— (eg L8), —(CH₂)ₘ₁—S—S—(CH₂)ₘ₂— (eg L9), —(CH₂)ₘ₁—N-succinimide-3-S(CH₂)ₘ₂— (eg L12), —C(O)-(4-cyclohexyl)-CH₂—N-succinimide-3-S(CH₂)ₘ₂— (eg L11), —(CH₂)ₘ₁—S—CH₂C(O)—(CH₂)ₘ₂— (eg L10), with m1 and m2, identical or different, being integers chosen in the range 0 to 6. For clarity, those selected examples of preferred linkers L1-112 are drawn below indicating the sites where the two moieties M1 and M2 should be attached; either of these moieties may be the azoaryl moiety, the other moiety is then the reporter. Such linkers are abbreviated in the text as -L-, thus a linked construct is represented as M1-L-M2. For unsymmetrical linkers, this is intended to cover both left-to-right and right-to-left orientations of the linker, ie. M1-L-M2 and M2-L-M1.

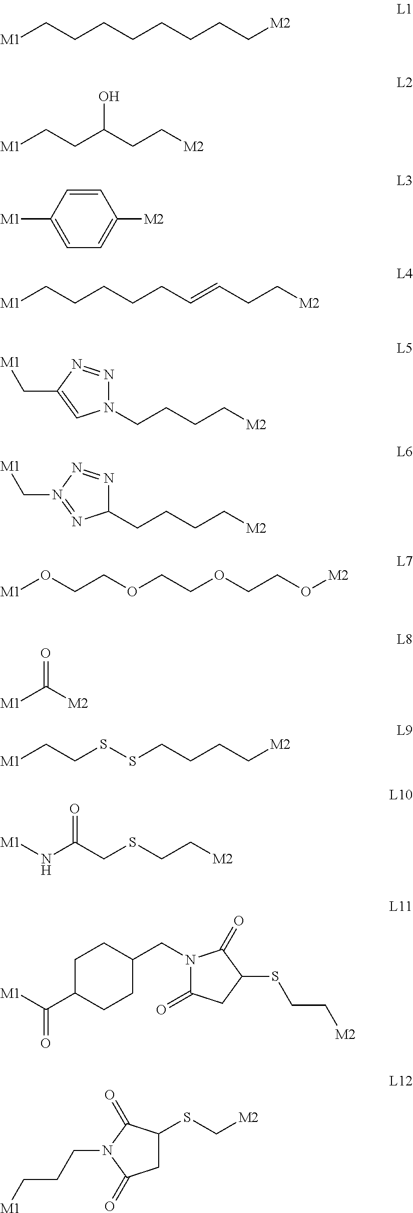

A “cleavable group” is intended to mean a group which (i) may be attached to an oxygen (in an alcohol or phenol), nitrogen (in an amine or aniline) or sulfur (in a thiol or thiophenol) atom of a compound of the invention, and (ii) where this cleavable group may undergo a chemical, enzymatic or photochemical triggering reaction which is followed by a cascade of reactions that eventually release the compound of the invention as the free corresponding alcohol, phenol, amine, aniline, thiol or thiophenol. The cleavage, and so the elimination of the cleavable group leading to an —NH, —OH or —SH function, is activated by a chemical, enzymatic or photochemical stimulus. A compound of the invention bearing a cleavable group may therefore function as a prodrug. In particular, the cleavage may occur in vivo, after the administration of the compound to a patient, or in vitro or in cellulo, depending on the application of the compound.

The term “prodrug” as used herein refers to any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically active compound. In particular, compounds of Formula (I), (B) or (A) are prodrugs when they include a cleavable group. The man skilled in the art will recognize that substituents and other moieties of the compounds of Formula (I), (B) or (A) should be selected in order to provide a compound which is sufficiently stable to provide a pharmaceutically useful compound which can be formulated into an acceptably stable pharmaceutical composition.

The term “treatment” denotes any therapeutic measure which is prophylactic or which suppresses a disease or disorder resulting in a desirable clinical effect or in any beneficial effect, including in particular the suppression or the reduction of one or more symptoms, or the regression, slowing down or cessation of the progression of the disorder which is associated therewith.

The expression “therapeutically effective amount” denotes any amount of a composition which improves one or more of the characteristic parameters of the affection treated.

The invention proposes to use compounds according to formula (I), (B) or (A) specifically for their capacity to be reversibly isomerised from their trans (E) to their as (Z) form upon exposure to light. The as form can be reconverted to the trans form by exposure to light or by spontaneous thermal conversion independent of light (eg. in the absence of an efficiently-absorbed wavelength or in the dark).

The key to the desirable properties of the present invention is that the cis and the trans forms of the same compound (I) have significantly different biochemical activity. The as form (I-cis) can directly present a tubulin polymerisation inhibitory activity and is named the active form; this is by contrast to its corresponding trans form (I-trans), named the inactive form, which does not present a significant tubulin polymerisation inhibitive activity when at a similar concentration as is needed to show this effect for (I-cis). It is also possible that structures (I-cis) feature a significant increase in tubulin polymerisation inhibitory activity after modification in vivo of one of the substituents, when the compound (I) acts as a prodrug. The invention therefore achieves novel inhibitors of tubulin polymerisation which can be fully reversibly switched between strongly and very weakly inhibitory forms in a predictable, practical fashion, such that these inhibitors possess distinct functional advantages over current antitubulin/antimitotic/antiproliferative/vascular disrupting/antiangiogenic/chemotherapeutic agents. Scheme 1 hereafter illustrates the principle used in the invention, for the case of compound I.1.

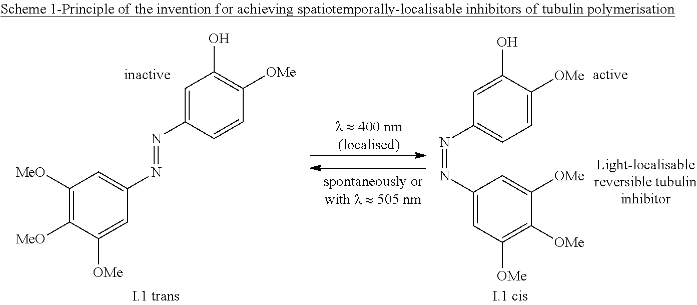 [SMILES: COc1ccc(/N=N/c2cc(OC)c(OC)c(OC)c2)cc1O ; COc1ccc(/N=N\c2cc(OC)c(OC)c(OC)c2)cc1O]

According to the invention, it was demonstrated that the azoaryls according to formulae (I), (B) and (A) are active as tubulin polymerisation inhibitors, directly or after modification of one of their substituents in vivo, in a cis form of the diazenyl bond and inactive in the corresponding trans isomeric form. This demonstration was given for several compounds according to the invention, and the invention validates that the azoaryls according to formulae (I), (B) and (A) can display similar biological activity to that known for their closely isosteric analogues, which are the known pharmacophore nuclei of the combretastatin family of colchicine-domain tubulin binding agents.

So, in general, the preferred embodiments for most compounds (I), (B) and (A) follow the well-known literature of the SAR of the combretastatin family. As a result, the compounds (I), (B) and (A) have preferably one of the following characteristics, any combination of the following characteristics, or all the following characteristics, when they do not exclude each other:
R₂ and R₃ are chosen separately among —OCH₃, —OCF₃, —F, —CH₃, —CF₃, —CH₂CH₃ and —OCH₂CH₃; R₂ and R₃ are —OCH₃ preferably;
R₂, R₃, R₄, and R₅ are chosen separately among —OCH₃, —OCF₃, —F, —CH₃, —CF₃, —CH₂CH₃ and —OCH₂CH₃;
R₂═R₃═R₄═R₅═OCH₃;
R₂═R₃═R₄═R₅═OCH₃; R₆, and when X₁═C(R₇), R₇ also, are chosen separately among —H, —F, —Cl, —NHCOCH₃, —N(CH₃)₂, and —OCH₃; and R₈═R₉═R₁₀═H;
R₆, and when X₁═C(R₇), R₇ also, are chosen among —H, —F, —Cl, —NO₂, —NHCOCH₃, —N(CH₃)₂, and —OCH₃; R₆═H preferably;
R₈, and if present then R_{g} and R₁₀ also, are hydrogen;
Z═C(R₁) with R₁═Y₁Rₐ, with Y₁═O, NH or S and with Rₐ being as defined for formula (I).

For the compounds which are the pharmacologically most active, usually Rₐ═H, but Rₐ≠H may allow advantageous tuning of solubility, biodistribution, and enable prodrug targeting, as is well established in the literature for other compounds bearing these functional groups^{[18,19]}.

R₆ and R₇ can be chosen to modify photoisomerisation, biodistribution or solubility parameters, which may be especially advantageous when R₁ is chosen as —H, —OH, —NH₂, —F or —SH. Indeed, a relatively large variation in the choice of R₆ and/or R₇ will be well tolerated while maintaining satisfactory biological effects, since these groups are not on the binding face of the azoaryl moiety. Therefore, these groups may preferentially be chosen to increase solubility, to effect biological targeting via prodrug strategies, or to lead to optimised photoisomerisation properties. In general, τ (the halflife of spontaneous cis->trans isomerisation) affects the required dosage and irradiation schedule in an experiment; some applications in research or medicine would be best with short τ (eg 1-10 minutes), but some with longer (eg 2-48 hours) T. Some variation of the R₆ and/or R₇ groups (ie. R₆ and/or R₇≠H) can be made, while leaving the other substituents untouched, to tune τ while keeping an approximately similar biological activity. This is illustrated in the Examples. The efficiencies and completeness of trans->cis and especially of cis->trans photoisomerisations are also important. A preferred compound according to the purposes of the current invention would be one that is rapidly cis->trans photoisomerisable at a certain “photorelaxation” wavelength giving nearly 100% of the trans form, as well as efficiently trans->cis photoisomerisable at a given “photoactivation” wavelength giving a substantial percentage (eg >70%) of the cis form. This is because, if photorelaxation leaves a substantial proportion of the sample in the as form (ie. the cis->trans photoisomerisation is not quantitative), then in some applications and also depending on τ, there may be residual toxicity after photorelaxation (due to residual cis isomer) even where and/or when none is desired. In order to reduce this residual toxicity by pursuing more quantitative photorelaxation, modifications can be made to the structures and again this may preferentially be performed at the R₆ and/or R₇ positions, as is illustrated in the Examples. The wavelengths of trans->cis and cis->trans photoisomerisations are also important. A major goal in biomedical applications is to obtain photopharmaceutical compounds which may be photocontrolled by single-photon absorption at long wavelengths such as λ>600 nm, to ensure maximum scope and accuracy of applications in deep tissues. These positions R₆ and/or R₇ can be used to pursue such “spectral red-shifting” (increasing the wavelengths of absorption) and this is illustrated in the Examples. Thus these positions R₆ and/or R₇ can be used to pursue spectral red-shifting, and/or to modify halflife τ, and/or to favour solubility, and/or to achieve prodrug targeting, or to connect to another functional moiety which is preferentially a reporter as defined in the invention, via a linker as defined in the invention. Linkers may preferentially be attached to an azoaryl moiety by eg. alkylation or acylation reactions of R₆ and/or R₇ when R₆ and/or R₇═OH or NH₂, or by a “click” reaction as is well understood by the man skilled in the art, when either an azide or alkyne-bearing functionality is attached at one of these positions.

In particular these considerations of R₆ and R₇ lead to preferred embodiments where X1=C(R₇) with R₇═—Y₂R_{f}, with Y₂═O, NH or S, and with R_{f} as defined for formula (I); and/or where R₆═—Y₂R_{f} with Y₂═O, NH or S, and with R_{f} as defined for formula (I); and/or where X1=C(R₇) with R₇═—NH-peptidic group; and/or where R₆═—NH-peptidic group; and/or where R₆ is a linker-reporter unit -Link1-Rep1 and/or X1=CR₇ in which R₇ is a linker-reporter unit -Link2-Rep2 where:
the reporters Rep1 and Rep2, identical or different, are chosen as fluorophores, chromophores, antennas or tag moieties, and especially among fluorescein, rhodamine, coumarin, phenoxazine, acridine, boron-dipyrromethene, dansyl, propidium, nitrobenzofurazan, resorufin, cyanine, Cascade Yellow, Nile Red, carborhodamine, silarhodamine, DABCYL, black hole quencher moieties, (E)-4,4′-bis(diethylamino)stilbene, biotin, substrates for tag proteins such as “SNAP-tag” (eg. substrate O⁶-benzylguanine), “CLIP-tag” (eg. substrate O²-benzylcytosine), “Halo-tag” (eg. substrate —((CH₂)₂O)₂(CH₂)₆Cl); and their derivatives, and
the linkers Link1 and Link2, identical or different, are chosen among bivalent (C₁-C₁₂)alkyl; bivalent (C₁-C₁₂)alkenyl; —(CH₂)ₘ₁(C₃-C₇)cycloalkyl(CH₂)ₘ₂—; —(CH₂)ₘ₁aryl(CH₂)ₘ₂—; moieties including between 1 to 10 carbon atoms and 1 to 6 heteroatoms chosen from among oxygen, nitrogen and sulfur, such as —(CH₂)ₘ₁heteroaryl(CH₂)ₘ₂— especially when heteroaryl is a triazole, tetrazole or pyridazine, —(CH₂)ₘ₁heterocycle(CH₂)ₘ₂—, oligo(ethyleneglycol), —(CH₂)ₘ₁—C(O)O—(CH₂)ₘ₂—, —(CH₂)ₘ₁—C(O)NH—(CH₂)ₘ₂—, —C(O)—, —(CH₂)ₘ₁—S—S—(CH₂)ₘ₂—, —(CH₂)ₘ₁—N-succinimide-3-S(CH₂)ₘ₂—, —C(O)-(4-cyclohexyl)-CH₂—N-succinimide-3-S(CH₂)ₘ₂— and —(CH₂)ₘ₁—S—CH₂C(O)—(CH₂)ₘ₂— with m1 and m2, identical or different, being integers chosen in the range 0 to 6.

Prodrug strategies are used especially when Z═C(R₁) with R₁═Y₁Rₐ and Rₐ being a cleavable group; and/or when X1=C(R₇) with R₇═—Y₂R_{f} and R_{f} being a cleavable group; and/or when R₆═—Y₂R_{f} with R_{f} being a cleavable group.

A “cleavable group” is intended to mean a group which (i) may be attached to an oxygen (in an alcohol or phenol), nitrogen (in an amine or aniline) or sulfur (in a thiol or thiophenol) atom of a compound of the invention, and (ii) where this cleavable group may undergo a chemical, enzymatic or photochemical triggering reaction which is followed by a cascade of reactions that eventually release the compound of the invention as the free alcohol, phenol, amine, aniline, thiol or thiophenol. A compound of the invention bearing a cleavable group may therefore function as a prodrug, which is an especially preferred embodiment of the invention.

Cleavable groups may contain one or two sequentially-attached subunits chosen, identical or different, from among cyclisation spacers, elimination spacers, or photolabile protecting groups. When a cleavable group contains one subunit, this is then called the upstream subunit (abbreviated U-); when a cleavable group contains two subunits, the subunit directly connected to the azoaryl moiety of the compound of the invention is called the downstream spacer, and the other subunit is called the upstream subunit (U-).

The cleavage of the whole cleavable group is initiated following a triggering reaction which acts on the upstream subunit; a cascade of reactions follows which liberates the leaving group of the upstream subunit. When a cleavable group contains one subunit, the leaving group is preferably a phenol, aniline or thiophenol group of the azoaryl moiety of a compound of the invention according to formula (I), (B) or (A). When a cleavable group contains two subunits, the leaving group of the upstream subunit is (following appropriate protonation) the triggered functional group of the downstream spacer; this downstream spacer then undergoes a further cascade of reactions to liberate the azoaryl moiety of a compound of the invention (I); either cyclisation or elimination spacers may be used as downstream spacers. It will be recognised by those skilled in the art that such concatenations (upstream subunit-downstream spacer-compound) are in regular use as “chemical adaptor systems”^{[20]}. The leaving group (abbreviated LG for clarity in chemical descriptions) of either the upstream subunit or of a downstream spacer may in general be an alcohol, phenol, amine, aniline, thiol or thiophenol. Thus the upstream subunit connected to its leaving group may be abbreviated U-LG.

Three families of cleavable group subunits are intended, each of which contains between 3-12 carbons and between 2-12 heteroatoms chosen, identical or different, among O, N, P and S, subject to the restrictions given below. These families are illustrated and described further, below. Note that the cleavage reaction descriptions below all implicitly assume appropriate protonation from the biological medium as required, which will be clear to those skilled in the art. Different examples of the cleavable group subunits that can be used in the invention are described in the literature cited hereafter and can be represented as follows:

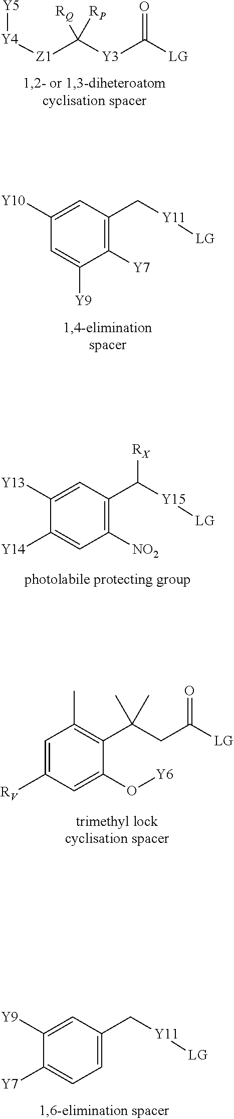

The first subunit family are 1,5- and 1,6-cyclisation spacers. Cyclisation spacers are either substituted 1,2- and 1,3-diheteroatom cyclisation spacers, which as upstream subunits may be directly triggered by peptidases^{[21,22]} or disulfide reductases^{[23]}, or else are trimethyl lock cyclisation spacers, which as upstream subunits may be directly triggered by esterases, glycosidases or phosphatases^{[24]}; both types may also be used as downstream spacers. Cyclisation spacers act following a triggering reaction which unmasks, as the triggered functional group, a thiol, amine or alcohol; this group then performs an intramolecular attack onto a substituted carbonyl group bearing LG via a five-membered or six-membered cyclic transition state, which results in the release of LG.

1,2- and 1,3-diheteroatom cyclisation spacers have the general formula LG-C(O)—Y3-C(R_{P})(R_{Q})—Z1-Y4-Y5, where —Z1- is —CH(R_{S})— in the case of 1,2-diheteroatom-ethanes, or —C(R_{T})(R_{U})—CH(R_{S})— in the case of 1,3-diheteroatom-propanes. In either case, the triggered functional group is —Y4H. Their mechanisms of releasing LG are illustrated below:

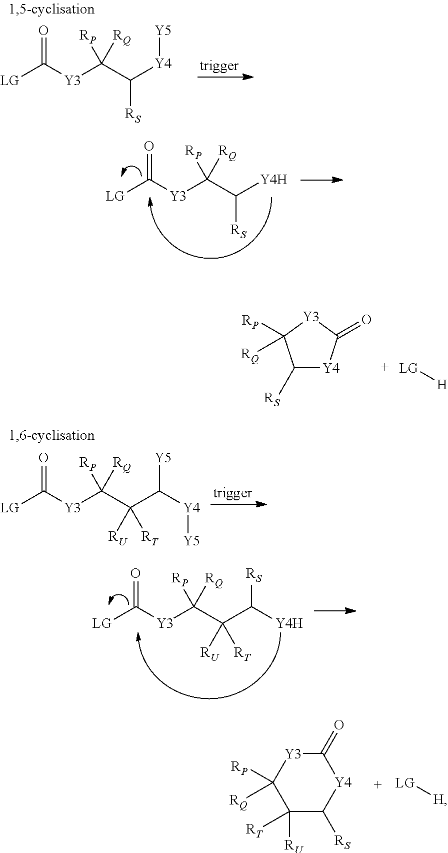

wherein:

Y3 is O or NR_{W};

Y4 is O, NH or S;

R_{Q}, R_{T} and R_{U} are chosen, identical or different, as H or CH₃;

R_{P}, R_{S} and R_{W} are chosen, identical or different, as H or CH₃ or else such that, if present, R_{Y} is connected to R_{S} as outlined below, or else such that, if present, R_{W} is connected to R_{P} by groups —(CH₂)₂— or —(CH₂)₃— such that a five- or six-membered ring is formed including these positions^{[22]};

when Y4=NH, then Y5 is either a peptidic group (in the case of an upstream subunit defining a peptidase-triggered cleavable group), or else Y5 is -U (in the case of a downstream spacer);

when Y4=O, such spacers are suitable only as downstream spacers, so Y5 is O-U;

when Y4=S, Y5 is either -U (in the case of a downstream spacer), or else Y5 is SR_{Y} (in the case of an upstream subunit defining a disulfide reductase-triggered cleavable group) where R_{Y} is a saturated group comprising between 1 and 8 carbons and between 0 and 2 heteroatoms chosen, identical or different, among O and N, and where preferably position R_{Y} is connected to R_{S} by bridging groups —(CH₂)₂— or —(CH₂)₃— such that a five- or six-membered ring is formed including these positions^{[25]}, examples of which are shown below:

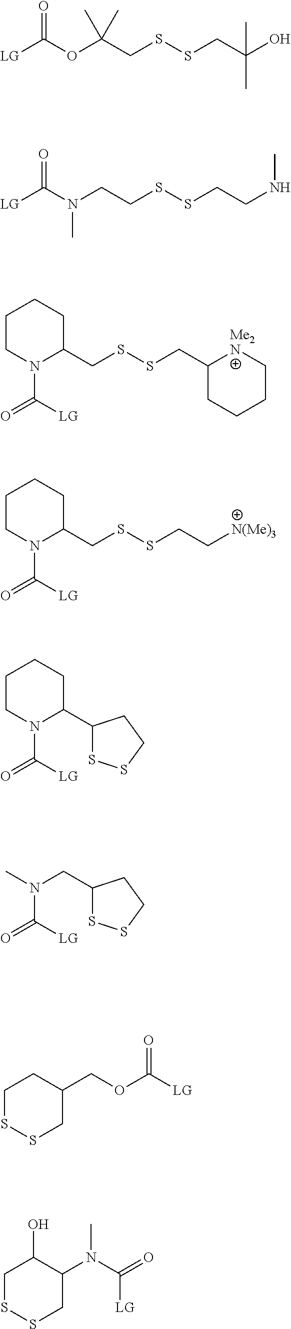

By way of example, compound I.10 features a cleavable group based around a 1,5-cyclisation spacer, triggered by the enzymatic action of in vivo peptidases, and releasing a phenolic compound of the invention, I.1, as the leaving group LG; the compound I.10 is further defined by Y3=NR_{W}, R_{Q}═R_{S}═H, Y4=NH, Y5=L-leucyl (Leu-), and substituent pair (R_{P} and R_{W}) being connected by a polymethylene bridge such that a 6-membered ring is formed including these positions; and the amine terminus is in the ammonium salt form (trifluoroacetate counterion).

Cleavable group subunits based around trimethyl lock cyclisation spacers have the following structure and mechanism of action (note that their triggered functional group is the phenolic hydroxyl group):

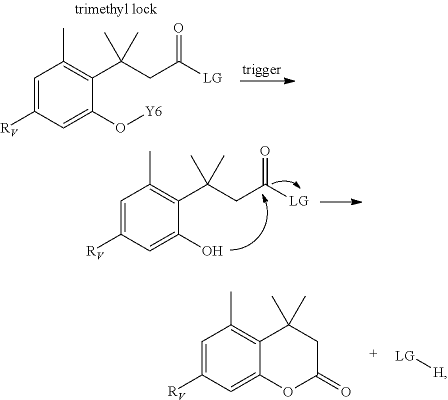

wherein:

R_{V} is chosen as —H or —CH₃;

when the trimethyl lock spacer is triggered by phosphatases, then Y6 is chosen as —PO₃H₂ or salts thereof such as —PO₃Na₂;

when the trimethyl lock spacer is triggered by glycosidases, then Y6 is chosen as a -glycosidyl group;

when the trimethyl lock spacer is triggered by esterases, then Y6 is chosen as an acyl unit —COR_{Z} or an acyloxymethyl unit —OCH₂OC(O)R_{Z} where R_{Z} is chosen among (C1-C6)alkyl, (C1-C6)alkenyl, (C1-C6)alkynyl, (C3-C7)cycloalkyl, and (C1-C6)alkyl(C3-C7)cycloalkyl;

or else in the case of a downstream spacer, Y6 is U.

The second family of cleavable mom subunits are elimination spacers. These spacers act following a triggering reaction which generates as the triggered functional group (—Y8H) a thiophenol, aniline or phenol. This group then performs a 1,4- or 1,6-elimination reaction which expels the leaving group LG, thereby forming an ortho- or para-quinone methide, respectively (or their heteroarylic analogues).^{[26]} The structures and functional mechanisms of elimination spacers are depicted below.

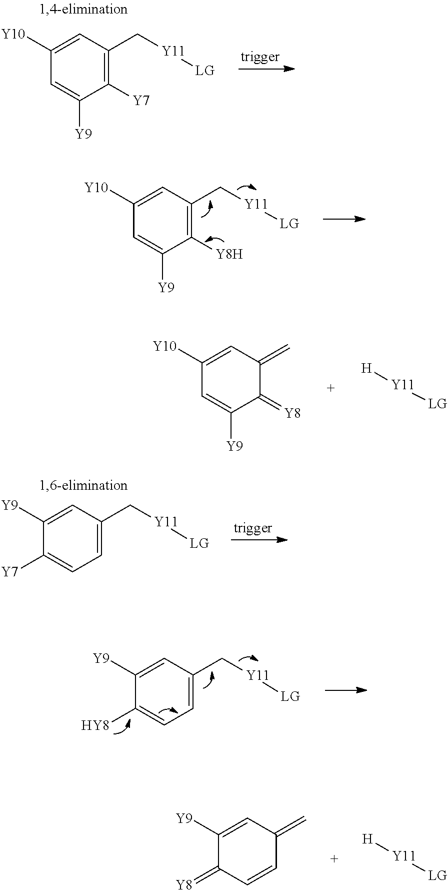

in the case of elimination spacers triggered by nitroreductases, Y7 is —NO₂, in which case Y8 is —NH, and Y9 and Y10 (if present) are H;

in the case of elimination spacers triggered by phosphatases, Y7 is —OPO₃²⁻, in which case Y8 is —O, and Y9 and Y10 (if present) are H;

in the case of elimination spacers triggered by peptidases, Y7 is —NH-peptidic group with the peptidic group as defined above, in which case Y8 is —NH, and Y9 and Y10 (if present) are H;

in the case of elimination spacers triggered by glycosidases, Y7 is —O-glycosidyl group, with glycosidyl group as defined above, in which case Y8 is —O, and Y9 and Y10 (if present) are chosen, identical or different, from H or —NO₂;

in the case of an elimination spacer used as a downstream spacer, then abbreviating the upstream spacer as U, either Y7=—NH-U (in which case Y8=NH), or else Y7=—OU (in which case Y8=O); and in both cases, Y9 and Y10 (if present) are H;

Y11 is used to indicate that either a direct single bond may join the benzylic CH₂ group to the group LG, or that, if LG is an amine or aniline group, then Y11 may advantageously be chosen as a group —O—C(O)— which connects them. When Y11=—OC(O)—, H-LG is obtained after spontaneous elimination of CO₂.

The third family of cleavable group subunits covers photolabile protecting groups, which may only be used as upstream subunits. Their light-induced cleavage is typically based on the shift of a light-generated radical from an aromatic nitro group to a group ortho to it following triggering illumination. Photolabile protecting groups useful for the current invention are those based on the 2-nitrobenzyl group^{[27]}, such as the 4,5-dimethoxy-2-nitrobenzyl and 4,5-dimethoxy-2-nitrobenzyloxycarbonyl groups; these may be triggered to release leaving groups LG when in the following structure:

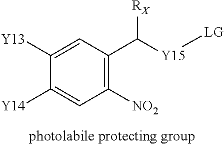

wherein:

Y13 and Y14, identical or different, are chosen as H or OCH₃ or else Y13-Y14 may be a linked system —OCH₂O—;

R_{X} is chosen among H, CH₃, C(O)CH₃ and COOH;

and Y15 is used to indicate that either a direct single bond may join the group CH(R_{X}) to the group LG, or that, if LG is an amine or aniline group, then Y15 may advantageously be chosen as a group —O—C(O)— which connects them. When Y15=—OC(O)—, H-LG is obtained after spontaneous elimination of CO₂.

Hereafter, are given specific examples of compounds according to the invention:
2-methoxy-5-((3,4,5-trimethoxyphenyl)diazenyl)phenol (I.1):

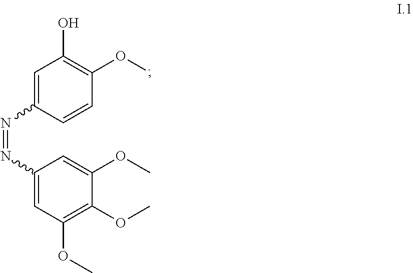

2-methoxy-5-((3,4,5-trimethoxyphenyl)diazenyl)aniline (I.2):

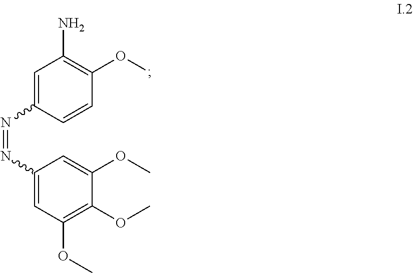

1-(3-fluoro-4-methoxyphenyl)-2-(3,4,5-trimethoxyphenyl)diazene (I.3):

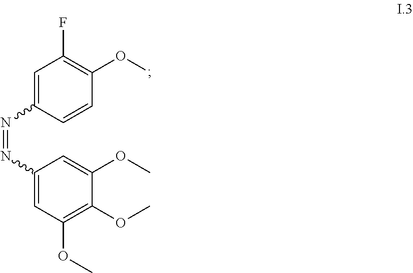

1-(2-fluoro-4-methoxyphenyl)-2-(3,4,5-trimethoxyphenyl)diazene (I.4):

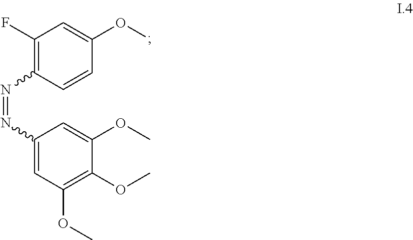

1-(2,3-difluoro-4-methoxyphenyl)-2-(3,4,5-trimethoxyphenyl)diazene (I.5):

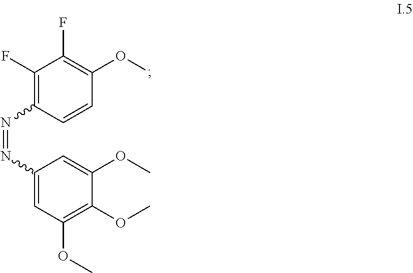

1-(2-fluoro-4-methoxy-3-nitrophenyl)-2-(3,4,5-trimethoxyphenyl)diazene (I.7):

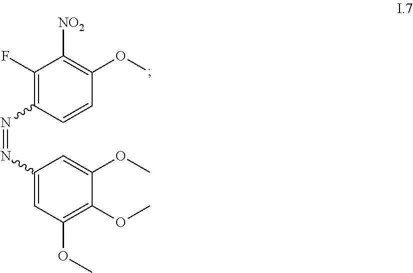

5-methoxy-2-((3,4,5-trimethoxyphenyl)diazenyl)phenol (I.8):

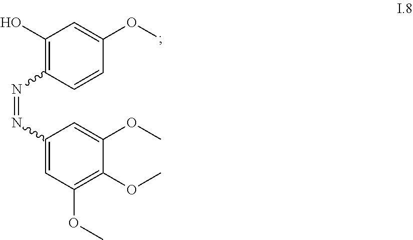

1-(3,5-difluoro-4-methoxyphenyl)-2-(4-methoxyphenyl)diazene (I.9):

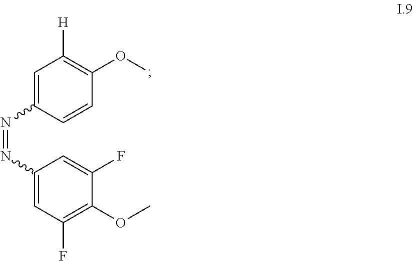

(2-methoxy 5-((3,4,5-trimethoxyphenyl)diazenyl)phenyl) 2-((L-leucinamido)methyl)piperidine 1-carboxylate 2,2,2-trifluoroacetate salt (I.10):

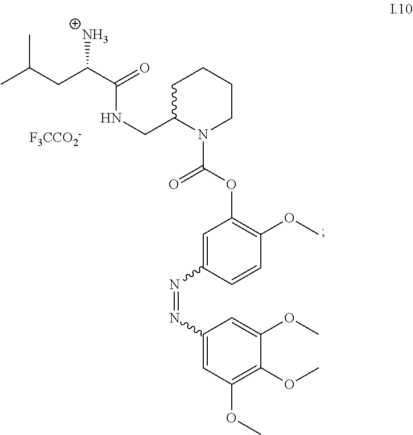

and its free form,
1-(3,4-dimethoxyphenyl)-2-(3,4,5-trimethoxyphenyl)diazene (I.11):

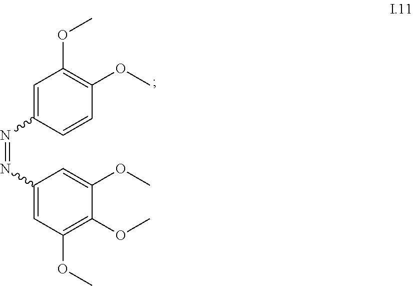 [SMILES: COc1ccc(N=Nc2cc(OC)c(OC)c(OC)c2)cc1OC]

1-(2,4-dimethoxyphenyl)-2-(3,4,5-trimethoxyphenyl)diazene (I.12):

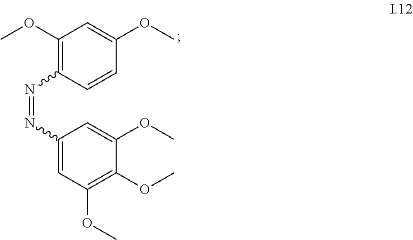

2-methoxy-5-((3,4,5-trimethoxyphenyl)diazenyl)pyridine (I.13):

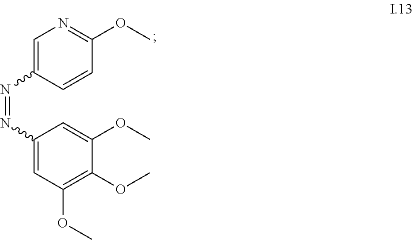 [SMILES: COc1ccc(N=Nc2cc(OC)c(OC)c(OC)c2)cn1]

8-methoxy-5-((3,4,5-trimethoxyphenyl)diazenyl)quinoline (I.14):

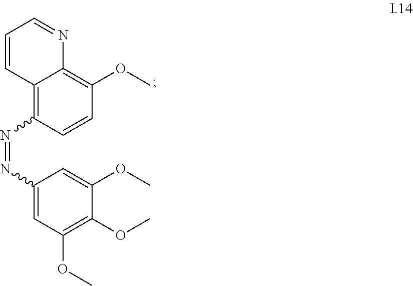 [SMILES: COc1cc(N=Nc2ccc(OC)c3ncccc23)cc(OC)c1OC]

methyl 8-methoxy-5-((3,4,5-trimethoxyphenyl)diazenyl)quinoline-2-carboxylate (I.15):

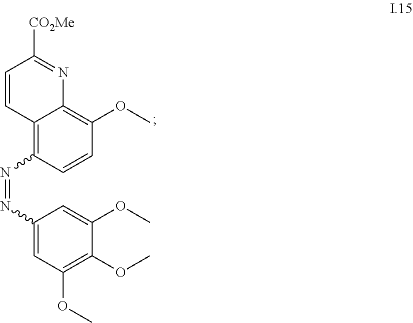 [SMILES: COC(=O)c1ccc2c(N=Nc3cc(OC)c(OC)c(OC)c3)ccc(OC)c2n1]

5-methoxy-2-((3,4,5-trimethoxyphenyl)diazenyl)aniline (I.16):

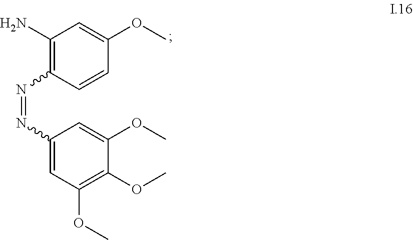 [SMILES: COc1ccc(N=Nc2cc(OC)c(OC)c(OC)c2)c(N)c1]

3-methoxy-2-methyl-6-((3,4,5-trimethoxyphenyl)diazenyl)phenol (I.17):

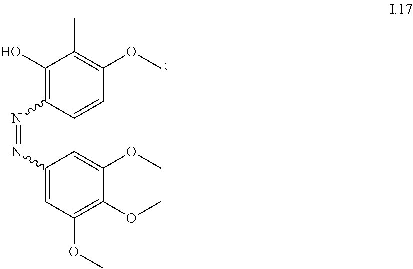 [SMILES: COc1ccc(N=Nc2cc(OC)c(OC)c(OC)c2)c(O)c1C]

2-(3-methoxy-2-methyl-6-((3,4,5-trimethoxyphenyl)diazenyl)phenoxy)ethan-1-ol (I.18):

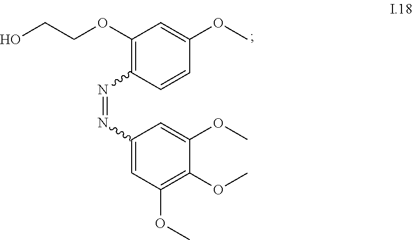 [SMILES: COc1ccc(N=Nc2cc(OC)c(OC)c(OC)c2)c(OCCO)c1]

2-(5-methoxy-2-((3,4,5-trimethoxyphenyl)diazenyl)phenoxy)ethan-1-ol (I.19):

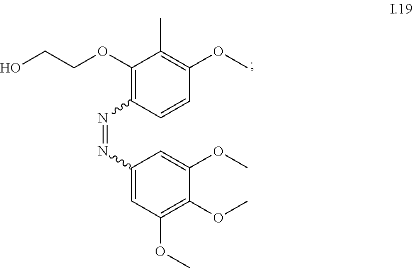 [SMILES: COc1ccc(N=Nc2cc(OC)c(OC)c(OC)c2)c(OCCO)c1C]

(2-methoxy 5-((3,4,5-trimethoxyphenyl)diazenyl)phenyl)) 1-L-serinamide 2,2,2-trifluoroacetate salt (I.20):

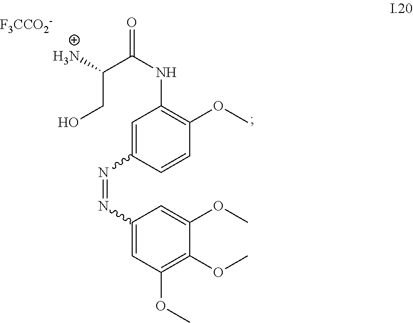 [SMILES: "F3CCO2-" ; COc1ccc(N=Nc2cc(OC)c(OC)c(OC)c2)cc1NC(=O)[C@@H]([NH3+])CO]

and its free form,
3-(3-methoxy-2-methyl-6-((3,4,5-trimethoxyphenyl)diazenyl)phenoxy)propan-1-ol (I.21):

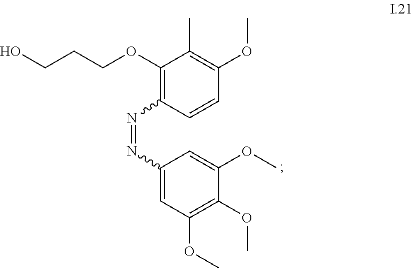 [SMILES: COc1ccc(N=Nc2cc(OC)c(OC)c(OC)c2)c(OCCCO)c1C]

2-methoxy-5-((3,4,5-trimethoxyphenyl)diazenyl)phenyl phosphate disodium salt (I.24):

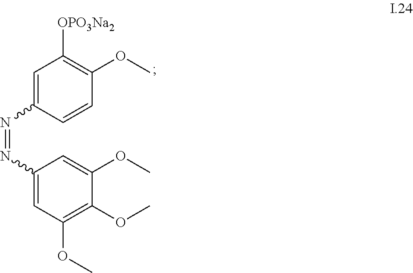

and its free form;
N-(6-(diethylamino)-9-(2-(4-(3-(3-methoxy-2-methyl-6-((3,4,5-trimethoxyphenyl)diazenyl)phenoxy)propyl)piperazine-1-carbonyl)phenyl)-3H-xanthen-3-ylidene)-N-ethylethanaminium bis(formate) salt (I.25):

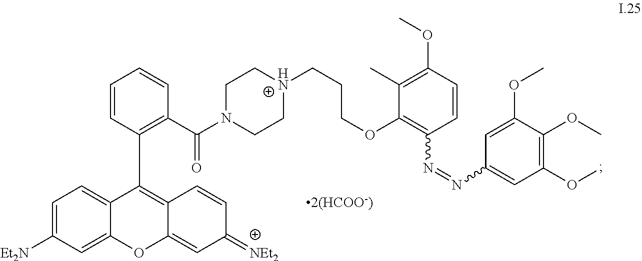

N-(5-methoxy-2-((3,4,5-trimethoxyphenyl)diazenyl)phenyl)acetamide (I.26):

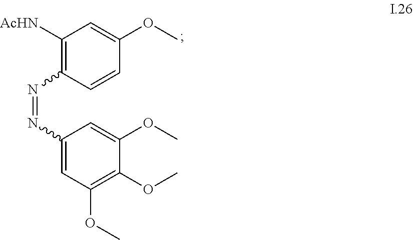 [SMILES: COc1ccc(N=Nc2cc(OC)c(OC)c(OC)c2)c(NC(C)=O)c1]

1-(3-bromo-4-methoxyphenyl)-2-(3,4,5-trimethoxyphenyl)diazene (I.27):

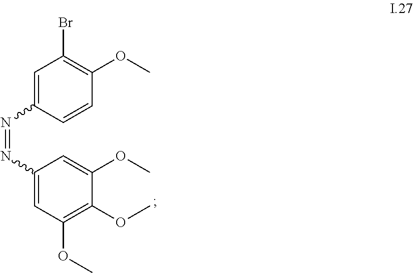 [SMILES: COc1ccc(N=Nc2cc(OC)c(OC)c(OC)c2)cc1Br]

2-methoxy-5-((3,4,5-trimethoxyphenyl)diazenyl)benzaldehyde (I.28):

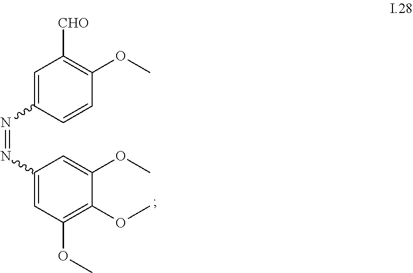 [SMILES: COc1ccc(N=Nc2cc(OC)c(OC)c(OC)c2)cc1C=O]

2-methoxy-5-((3,4,5-trimethoxyphenyl)diazenyl)benzoic acid (I.29):

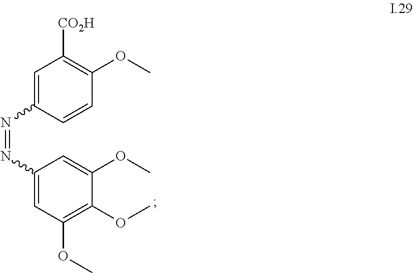 [SMILES: COc1ccc(N=Nc2cc(OC)c(OC)c(OC)c2)cc1C(=O)O]

and 1-(4-(trifluoromethoxy)phenyl)-2-(3,4,5-trimethoxyphenyl)diazene (I.30):

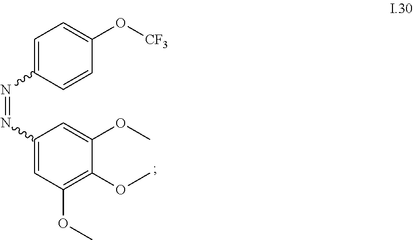 [SMILES: COc1cc(N=Nc2ccc(OC(F)(F)F)cc2)cc(OC)c1OC]

5-((3,5-dimethoxy-4-(trifluoromethoxy)phenyl)diazenyl)-2-(trifluoromethoxy)phenol;
2-(trifluoromethoxy)-5-((3,4,5-trimethoxyphenyl)diazenyl)phenol;
5-((3,5-dimethoxy-4-(trifluoromethoxy)phenyl)diazenyl)-2-methoxyphenol;
8-methoxy-5-((3,4,5-trimethoxyphenyl)diazenyl)quinoline-2-carboxylic acid;
2-fluoro-6-methoxy-3-((3,4,5-trimethoxyphenyl)diazenyl)aniline;
N-(2-hydroxy-3-methoxy-6-((3,4,5-trimethoxyphenyl)diazenyl)phenyl)acetamide;
5-((3,5-dimethoxy-4-(trifluoromethoxy)phenyl)diazenyl)-2-(trifluoromethoxy)phenyl dihydrogen phosphate;
2-(trifluoromethoxy)-5-((3,4,5-trimethoxyphenyl)diazenyl)phenyl dihydrogen phosphate;
5-((3,5-dimethoxy-4-(trifluoromethoxy)phenyl)diazenyl)-2-methoxyphenyl dihydrogen phosphate;
2-methoxy-1-methyl-5-((3,4,5-trimethoxyphenyl)diazenyl)pyridinium iodide;
2-methoxy-5-((3,4,5-trimethoxyphenyl)diazenyl)pyridine-N-oxide;
5-methoxy-2-((3,4,5-trimethoxyphenyl)diazenyl)pyrimidine; and
5-methoxy-4-methyl-2-((3,4,5-trimethoxyphenyl)diazenyl)pyrimidine; as a mixture of cis and trans isomers in any proportions and also as a pure isomer either cis or trans, and their hydrates, pharmaceutically acceptable salts and solvates.

Notable members of the combretastatin family currently in clinical trials include fosbretabulin (CA4P) which is a prodrug of combretastatin A-4 (CA4),^{[28]} ombrabulin which can be considered as a prodrug of a CA4 derivative in which the hydroxyl group is replaced by an amino group.^{[4]} Many other members and analogues of the combretastatin family also exhibit desirable inhibition of tubulin polymerisation.^{[1,2,28]} The invention gives examples of and/or proves the satisfactory biological effects of novel azoaryl analogues of several such validated antitubulin agents; eg. compound I.1-cis which is an azoaryl isostere of CA4, compound I.24-cis which is an azoaryl isostere of CA4P, and compound I.20-cis which is an azoaryl isostere of ombrabulin.

Note that the satisfactory biological activity of the compounds of the invention is assured by an appropriate substitution pattern, but it is also necessary to consider isomerisation parameters such as τ, PSS(λ) and ε(λ) especially for tuning preferred embodiments to different applications. Therefore the compounds of the invention are designed to include features which favour not only biological activity, but also which favour useful values of these isomerisation parameters. For example, fused-ring compounds I.14 and I.15 demonstrate significant absorption ε(λ) at λ>550 nm, as is desirable in the context of the invention.

The importance of the fact that the invention proposes cis-active azobenzene analogues must be stressed, since this makes them well-adapted for spatiotemporally targeted applications which minimise off-target biological effects. For cis-active azoaryl compounds such as those of the invention, only the targeted region requires illumination in order to locally generate a concentration of an active cis form sufficient to give the desired effects, and this is practical to achieve for the desired applications of the invention. Critically, elsewhere, where no illumination is applied, such molecules of the invention as diffuse out of the illuminated target zone in their active (cis) form will revert totally to the trans isomer by spontaneous isomerisation; and molecules which were never exposed to illumination since they were never inside the target zone, remain fully in a trans, thus inactive, form. Therefore the cis-active azoaryl compounds of the invention may present a practical method to achieve strictly on-target localisation of biological effects.

Tubulin polymerisation inhibitory activity can be shown and quantified according to many known methods^{[28,29]}, several of which are described in the examples. A compound is considered “active” as regards a tubulin polymerisation inhibitory effect if under any conditions of irradiation, (a) its IC₅₀ by tubulin polymerisation assay is less than 50 μM (with the IC₅₀ being defined as the concentration giving a 50% reduction in either net tubulin polymerisation level at the endpoint of an assay, or maximal rate of tubulin polymerisation, relative to a control without any applied compound); or (b) its EC₅₀ (or IC₅₀) by any cell-based assay is less than 50 μM; where the EC₅₀ (or IC₅₀) is defined as the concentration resulting in either (i) a 50% inhibition, relative to the control, of a measurement of normal cellular function, such as cell proliferation as measured by MTT assay^{[30]} or crystal violet staining^{[31]}; or else (ii) a 50% increase from the control level towards the maximal level of a quantity indicative of abnormal function, such as the percentage of cells arrested in G₂/M phase, or the percentage of apoptotic cells in subG1 phase, or the percentage of membrane-permeable cells, as determined by propidium iodide staining and flow cytometry analysis. The preferred method for determining if a compound is “active” or not, as regards a tubulin polymerisation inhibitory effect, is here considered to be flow cytometry-based analysis of cell cycle arrest in the G₂/M phase.

It is well known for the combretastatin family, as well as for other families of inhibitors of tubulin polymerisation dynamics such as taxanes and Vinca alkaloids, that inhibition of tubulin polymerisation dynamics can directly cause or is strongly correlated to a range of biologically and medically desirable effects in cellulo, in vitro and in vivo, including but not limited to cytotoxic, antiproliferative, antimitotic, antitumour, antivascular, antiangiogenic, vascular disrupting and/or antimetastatic effects (separately and collectively referred to as “biological effects”).^{[1,2,28]}

The present invention demonstrates that the compounds of formula (I) or (B), and in particular the azobenzene analogues (A) of combretastatins, can display similarly desirable biological effects as do the “parent” stilbenoid family of combretastatins, but with the important addition of the possibility of fully reversible spatiotemporal control over these biological effects via controlling an applied irradiation regime.

It is well demonstrated that light of defined wavelength, duration, intensity, and exposure pattern (referred to as “appropriate irradiation”) can easily be applied to a cuvette, subcellular region, cell, tissue, tumour zone, organism or other region of interest (separately and collectively referred to as “targets”) by technologies of light sources (including lamps, light-emitting diodes (LEDs), organic LEDs (OLEDs), lasers, and monochromators) which may be coupled with methods for light focussing and delivery (including endoscopes and fibre optic cables, optical table setups, microscopy methods including confocal and spinning disk microscopy) in a manner which is well-defined in time and space.

Therefore, it is possible to use spatiotemporally defined appropriate irradiation on targets where the compounds of formula (I), (B) or (A) described in the present invention are either locally or systemically administered or introduced. This allows compounds of formula (I), (B) or (A) described in the present invention to spatiotemporally influence biological effects on targets, such as tubulin polymerisation, cytotoxicity, anti-angiogenic activity, vascular flow in tumours, or antimitotic activity, in the subcellular region, cell, tissue, tumour or organism. Therefore the compounds of formula (I), (B) or (A) described in the present invention can be considered as reversibly modulatable tubulin polymerisation inhibitors, cytotoxins, antiproliferatives, antimitotics, antiangiogenic agents, chemotherapeutics and/or vascular disrupting agents, with the possibility of vastly reduced off-target biological effects.

The invention also addresses further challenges facing the state of the art in photoisomerisation-based targeting of inhibitors of tubulin polymerisation, as were outlined above for the research by Bisby, Scherer, Hadfield and McGown into stilbenoid compounds.^{[8,9]} The present invention proposes azoaryl derivatives, such as are generally known to feature very quantum-efficient photoisomerisation upon light absorption, and which are generally also strongly-absorbing chromophores (large single-photon absorption coefficient, and some examples of satisfactory two-photon absorption). This allows low doses of light to be sufficient for bulk single-photon photoswitching, as well practical applications of two-photon photoswitching.^{[7,10,32,33]} Additionally, azoaryl compounds of formula (I), (B) or (A) described in the present invention (eg. I.1, I.2, I.3 and I.4) can perform single-photon trans->cis and cis->trans photoswitching efficiently (using relatively low light intensities) with irradiation in the near-UV-to-visible spectral region which is of interest for biological compatibility reasons (relatively long wavelengths)^{[10,32]}. The examples show that with compounds of formula (I), (B) or (A) described in the present invention, biologically well-tolerated and much less scattered/absorbed wavelengths may be used to give substantial conversion of the trans to the as isomer after a short period of irradiation with a power applied of only approximately 10 mWcm⁻², using an efficient and low-cost, low-complexity single photon process. The examples also show that relatively long wavelengths (thus well tolerated and well penetrating) may be used to efficiently give a net reduction of the percentage of as isomer present in a sample, eg. using the “rescue” regime described in the Examples. For example, compound I.1 may be photoisomerised from all-trans to approx. 50% as using 450 nm light, or to approx. 90% as using 390 nm light, but the subsequent application of 505 nm light returns the population to only approx. 20% cis. Compound I.25 provides still greater cis->trans performance, as it may be very rapidly photoisomerised from >70% as back to >99% trans using very low intensity light of 550 nm (see Examples for details).

These features of the azoaryl compounds of the invention are extremely advantageous for both biological research and medical applications, when compared to the characteristics of the prior art in stilbene isomerisation. Phototoxicity is generally undesirable for research applications and especially for therapeutic applications: and both short wavelengths such as λ<<390 nm, and high light intensities, may be phototoxic to targets by a variety of mechanisms.^{[10,32]} They may also be difficult and costly to apply: high intensity light typically requires a laser source which may be costly, and it may be difficult to deliver this high intensity especially in biomedical settings; and short wavelengths such as λ<<380 nm are very strongly absorbed and scattered by biological tissues which may make it complex and/or costly to deliver them locally at satisfactory intensity in biological settings, especially without exposing non-target zones to this irradiation.^{[10,32]} By contrast, the azoaryl compounds of formula (I), (B) or (A) described in the present invention may be used relatively easily and cheaply (no high-intensity source or laser required; relatively long wavelengths suffice); this may make them well-suited to a range of applications, eg. in research and medicine, which may profit from their spatiotemporally-localised photoisomerisation method for reducing off-target toxicity, while not incurring other significant mechanisms of toxicity such as phototoxicity.

Finally, the compounds of formula (I), (B) or (A) described in the present invention possess the crucial advantage that they may be reversibly cycled by the action of light and/or thermal reversion between their trans and as forms potentially thousands of times without significant loss of activity or photochemical degradation.

Therefore compounds of formula (I), (B) or (A) described in the present invention, may enable sophisticated pharmacological applications of their spatiotemporally-localisable biological effects in a highly practical and robust fashion.

It must also be stressed that the present invention has anticipated the problems of water solubility and bioavailability which have been a major hindrance to the transition of combretastatins from fundamental research into medical applications.^{[1,3,4]} Therefore the present invention explicitly provides strategies to address solubility, including applications of prodrug strategies for phenols (I.10, I.24) and anilines (I.20), demonstrating the feasibility of applying well-known prior art techniques of prodrug synthesis to azoaryls (I), (B) or (A) of the current invention, especially when such compounds (I), (B) or (A) are phenols, anilines and/or thiophenols.^{[34]} Advantageously, such prodrugs may still further increase the on-target specificity that the invention can provide, by permitting orthogonal dual targeting (both illumination-based spatiotemporal targeting of trans<->cis isomerisation, and eg. enzymatic activity-based targeting of prodrug activation) to further discriminate for target cells only. The present invention also explicitly provides an example of the application of a non-prodrug solubilising strategy employing a covalently-linked water-soluble cation which likewise increases the solubility of the azoaryl construct (compound I.25).

So, the compounds of formula (I), (B) or (A) described in the present invention will be useful as medicaments (referred to as “medical applications”), especially as anti-mitotic, anti-angiogenic, antitumoral or chemotherapeutic agents. They possess key advantages in comparison to standard drugs for these and similar applications, and in particular to inhibitors of tubulin polymerisation such as the members of the combretastatin family. Such standard drugs are either applied in, or else are converted by biochemical reaction in vivo to, an active form which remains in this active form whether it is located in the target or not, and which therefore can present well-known and often dose-limiting problems of systemic off-target biological effects such as toxicity.^{[2,6]} The compounds of formula (I), (B) or (A) described in the present invention may for example enable therapies giving reduced side-effects relative to therapies performed with standard drugs.

The compounds of formula (I), (B) or (A) described in the present invention can be administered to the patient in need thereof in their trans isomer (the inactive form, (I-trans)), or in a mixture of their trans and cis isomers (mixture of (I-trans) and (I-cis)), and then be isomerised by spatiotemporally localized illumination in the target to generate a therapeutic amount of the cis isomer (the active form, (I-cis)) therein or of another active cis form when one or more of the substituents are modified in vivo. Even if it is not preferred, the patient in need thereof can be directly treated with a compound (I-cis), eg. by a localized treatment such as by injection directly in a target, such as a tumour zone. The compounds of formula (I), (B) or (A) described in the present invention may also be administered as prodrugs, wherein certain substituent(s) may be modified in vivo, before or after photoisomerisation to the cis form, and where one or more tubulin polymerisation inhibiting cis forms result from the combination of trans->cis photoisomerisation and the in vivo modification(s).

Whichever isomeric form is administered, it is possible to reduce the toxic effect of the compound on non-target cells since the as isomer can be converted to the trans form in non-target cells by appropriate irradiation and/or by spontaneous thermal conversion. These processes may be used to establish a concentration gradient of the active cis form such that a high concentration is maintained in the target while a lower concentration is maintained outside the target, thereby restricting the biological effects of the compounds of the invention preferentially within the target.

Additionally, by contrast to P1, the compounds of formula (I), and in particular the compounds of formula (B) or (A), are designed to exploit the key structural features of the most powerful members of the well-known combretastatin family of inhibitors of tubulin polymerisation, so as to benefit from both their highly desirable biological properties and their extensive SAR research. The compounds of formula (I), (B) or (A) described in the present invention can display significantly stronger tubulin polymerisation inhibition strength in vitro than P1, which much more closely mimics that seen for the combretastatins^{[1]}, and the submicromolar IC₅₀ concentrations which were demonstrated for selected compounds of formula (I), (B) or (A) when applied in several cell-based assays underlines their practical possibilities in a true cellular setting as tools and therapeutics for biology and medicine.

Additionally, it is a key advantage of the compounds of formula (I) and in particular the compounds of formula (B) or (A) described in the present invention that they are biologically less active in the more stable azoaryl isomeric form (trans), so that they may be applied globally but only activated locally (in a spatiotemporal sense). This ensures that the present invention minimises off-target effects, as outlined above. By contrast, P1 was disclosed to be more active in its trans form; azobenzenes of such molecular structure are known by analogy with the extensive literature to be both more stable in their trans form and spontaneously converted to their trans form at an appreciable rate in relevant media;^{[7]} therefore, as discussed above, compounds such as P1 cannot obtain such on-target selectivity as is described by the cis-active compounds of the invention.

The present invention also presents important advantages of functional possibilities over the prior art stilbene methods of Bisby, Hadfield, McGown and Scherer, since stilbenes do not allow fully reversible, photostable, high-efficiency photoswitched cycling between cis and trans isomers, as discussed above. Such fully reversible photoswitching, which is necessary for sophisticated applications as are intended for this invention, is however demonstrated for compounds of formula (I) in the Examples, and this is in accordance with the substantial photoisomerisation literature for azoaryl compounds^{[7,32]}. Moreover, strategies for redshifting stilbenes' single-photon absorption from their ordinary absorption maxima around 310-340 nm to significantly longer (biologically compatible, eg. >400 nm) wavelengths are neither well-known nor demonstrated to be applicable to the combretastatin pharmacophore; therefore their prior art relied on multiphoton photoactivation which adds the complications as outlined above to the existing problems facing stilbene photoisomerisation. The azoaryl compounds of formula (I), (B) or (A) described in the examples feature strong single-photon absorption at biologically compatible wavelengths, as shown in the in vitro and in cellulo assays; moreover, certain compounds of formula (I), (B) or (A) described in the present invention also exploit strategies either known (for other azoaryl compounds^{[32,35]}) or, as far as can be ascertained, novel (eg. compound I.25) by which even longer wavelengths may efficiently achieve a significant impact on the ratio of trans to cis isomers.

As stated above, the invention also concerns the compounds as defined above, as medicaments, and in particular as anti-mitotic, anti-angiogenic, antitumoral or chemotherapeutic agents.

One particular objective of the invention is the compounds as defined above for their use in the treatment of a disease for which the administration of a compound with antitubulin activity has a beneficial effect, especially for their use in the treatment of a cancer, such as melanoma, adenocarcinoma of the lung, neuroblastoma, small cell carcinoma of the lung, breast carcinoma, colon carcinoma, ovarian carcinoma, or bladder carcinoma, or of a disease characterized by abnormal vascularisation such as diabetic retinopathy, psoriasis or endometriosis, or of rheumatoid arthritis or atherosclerosis, as such applications are known for other inhibitors of tubulin polymerisation, particularly those which may present anti-angiogenic effects such as the combretastatin family^{[36]}. The invention also concerns pharmaceutical compositions comprising a compound as defined above with at least one pharmaceutically acceptable excipient.

Another objective of the invention is a compound with an azoaryl structure for use in the treatment of a disease, especially of one of the diseases mentioned above, for which a tubulin polymerisation inhibitor activity has a beneficial effect, in which the compound is administered to the patient in need of such treatment, at least partially in its trans isomeric form of the diazenyl bond, and where this trans form is inactive as regards a tubulin polymerisation inhibitory effect, and where this trans form is isomerised in vivo to an azoaryl compound in its cis isomeric form of the diazenyl bond by the application of light, and where in vivo modification of one or more substituents occurs optionally, and either before or after this photoisomerisation, resulting in a cis form which is active as regards a tubulin polymerisation inhibitory effect.

According to one embodiment, the azoaryl compound is administered in its trans isomeric form of the diazenyl bond, and its cis form is active as regards a tubulin polymerisation inhibitory effect (ie. no in vivo modification of a substituent is required).

According to another embodiment, the azoaryl compound is administered in a mixture of cis and trans isomeric forms of the diazenyl bond, and its cis form is active as regards a tubulin polymerisation inhibitory effect.

The application of light is preferably localised (“appropriate irradiation” as defined above).

In a preferred way, the isomerisation in vivo of the diazenyl bond from trans->cis form is followed by cis->trans conversion by spontaneous thermal reversion or by application of light. This isomerisation in vivo of the diazenyl bond from the as to the trans form leads, advantageously, to an inactive form as regards a tubulin polymerisation inhibitory effect. The trans->cis and cis->trans conversions may optionally be repeated many times, and these conversions may preferably be localised differently in space and/or time.

The compound is preferably selected among the compounds in the trans form of the diazenyl bond, or in a mixture of cis and trans forms of the diazenyl bond, corresponding to formula (I), (B) or (A), and preferentially the compound is an azobenzene. The compound can also be 1-(4-methoxynaphthalen-1-yl)-2-(3,4,5-trimethoxyphenyl)diazene or 1-(4-methoxyphenyl)-2-(3,4,5-trimethoxyphenyl)diazene, either in the trans form or in a mixture of the trans and cis forms.

The compounds of formula (I), (B) or (A) described in the present invention are also tools for the study of the cytoskeleton (referred to as “research applications”), and may address needs that were impossible to meet with prior art systems, in particular for spatiotemporally defined studies of complex and dynamic phenomena. The compounds of formula (I), (B) or (A) described in the present invention will microscopically and macroscopically allow the spatiotemporally precise modulation of tubulin polymerisation and therefore of tubulin-dependent phenomena. Many of these phenomena have important applications to fundamental research including cell function, developmental biology, disease and therapy.^{[37]} For instance, organisms or cells in culture or on a microscope stage can be treated with a compound (I-trans); and (I-trans) may be converted to a biologically active as isomer (I-cis) or to other active cis forms when one or more of the substituents are modified in vivo, in all cells or organisms, in a subpopulation of cells or organisms, or in subcellular regions of interest such as around the chromosomes aligned within the metaphase plate or around the centrioles of a cell undergoing division; the cells can then be studied. Alternatively, the target to be studied can be directly treated with (I-cis), or a mixture of (I-cis) and (I-trans). After the desired phase of study, the active cis form(s) can also be reconverted to inactive trans form(s), which may reduce the biological effects upon the cells or organisms, and may for example now allow them to resume normal development, cell division, or motility. This may notably allow sophisticated modification of developmental biology, cell cycle, intracellular transport, and other cytoskeleton-dependent phenomena. This application is very interesting as, at this time, there is no method for rapid-response spatiotemporally-localised and/or reversible control of the cytoskeleton within cells. The most practical current method for modulating the concentration of the active form of a drug in cells is to add the drug into the cell culture or incubation media (raises intracellular concentration by inward diffusion), or else rinse the cells and apply new media without the drug (slowly lowers intracellular concentration by outward diffusion). However, (a) addition and especially rinsing methods rely in general on diffusion to transport the drug, thus to influence active concentrations within the target may require a far longer timescale than the characteristic times of dynamic biological processes such as mitotic cell cleavage; this prevents them from being suitable for dynamically reversible or cyclical modulation of drug concentration; (b) the intracellular concentrations thus generated are not quantitatively predictable, and in particular the increase or especially the decrease of intracellular drug concentration cannot be achieved sharply (in the manner of a step function); (c) diffusion always counteracts any targeting that may be achievable by localised drug application or unmasking; and crucially, since there is no general method appropriate to remove a diffusing active drug specifically from an off-target zone while not interfering with its concentration in a neighbouring on-target zone, therefore these prior methods cannot generally be used on a highly-localised scale to establish and maintain a concentration gradient of a drug so as to target subpopulations of cells, or especially to target subcellular regions within a single cell. So, the use of compounds according to the invention will offer new possibilities for the study of the cytoskeleton and its many functions and effects.

Another object of the invention therefore concerns a method of studying the cytoskeleton and/or its associated processes in which cells, and in particular tumoral cells, or an organism or sample are treated with an azoaryl compound, at least partially in its trans isomeric form of the diazenyl bond, where this trans form is inactive as regards a tubulin polymerisation inhibitory effect, and where this trans form is converted in vitro or in cellulo to an azoaryl compound in its cis isomeric form of the diazenyl bond which is active as regards a tubulin polymerisation inhibitory effect, by isomerisation in vitro or in cellulo of the diazenyl bond to its cis isomeric form by application of light, optionally with in vitro or in cellulo modification of one or more substituents.

According to one embodiment of this method, the azoaryl compound in its pure trans isomeric form of the diazenyl bond is the form of the compound used for treating the cells or the sample and its cis form is directly active as regards a tubulin polymerisation inhibitory effect.

According to another embodiment of this method, the azoaryl compound in a mixture of its cis and trans isomeric forms of the diazenyl bond is the form of the compound used for treating the cells or the sample and its cis form is active as regards a tubulin polymerisation inhibitory effect.

In this method, the application of light is preferably localised.

In a preferred embodiment of this method according to the invention, the conversion from the trans to the as form of the diazenyl bond is followed by its conversion from the cis to the trans form by spontaneous thermal reversion or by application of light with an appropriate wavelength which leads, advantageously, to an inactive form as regards a tubulin polymerisation inhibitory effect. The trans->cis and ds->trans conversions may optionally be repeated many times, and these conversions may preferably be localised differently in space and/or time.

The compound used in this method is preferably selected among the compounds in the trans form of the diazenyl bond, or in a mixture of cis and trans forms of the diazenyl bond, corresponding to formula (I), (B) or (A), and preferentially the compound is an azobenzene. The compound can also be the 1-(4-methoxynaphthalen-1-yl)-2-(3,4,5-trimethoxyphenyl)diazene or the 1-(4-methoxyphenyl)-2-(3,4,5-trimethoxyphenyl)diazene in its transform or in a mixture of its trans and cis forms.

The compounds used in the context of the invention may be prepared by adapting known conventional techniques for forming azobenzenes and/or their azoheteroaryl analogues^{[32,38,39]} (hereinafter referred to collectively and separately as azo compounds), along with such conventional techniques for modifying substituents on the thus-formed azo compounds or their precursors as may be found in the appropriate literature of azo compound chemistry or adapted from known reactions in eg. aromatic or heterocyclic chemistry, or in the chemistry of prodrug synthesis.

Compounds of formula (I) can be obtained following a reaction according to Scheme 2 below in which R′₂, R′₃, R′₄, R′₅, R′₆, R′₈, X′1, X′2 and Z′ are, respectively, R₂, R₃, R₄, R₅, R₆, R₈, X1, X2 and Z or precursors of the corresponding groups or the corresponding groups in a protective form regarding the conditions used for the synthesis of (IV) from (II) and (III). Compounds (IV) where R′₂═OH are often straightforward to form by this route. Therefore this synthesis is a preferred route towards compounds of structure (I) where R₂═OMe, as such compounds may straightforwardly be obtained from methylation of the precursor (IV) having R′₂═OH.

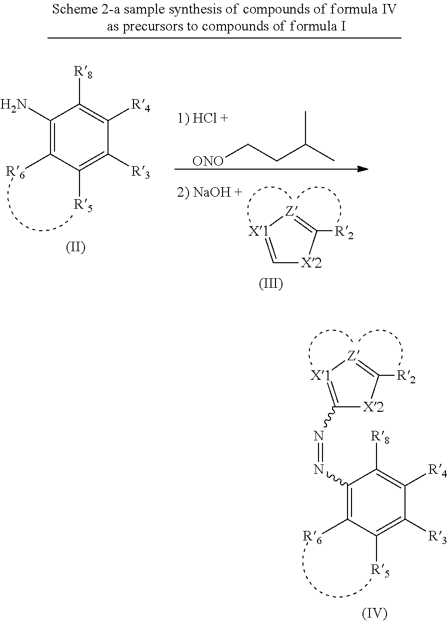

Alternatively, the compounds of formula (I) can be obtained following a reaction according to Scheme 3 below in which R′₂, R′₃, R′₄, R′₅, R′₆, R′₈, X′1, X′2 and Z′ are, respectively, R₂, R₃, R₄, R₅, R₆, R₈, X1, X2 and Z or precursors of the corresponding groups or the corresponding groups in a protective form regarding the conditions used for the synthesis of (IV) from (V) and (VI).

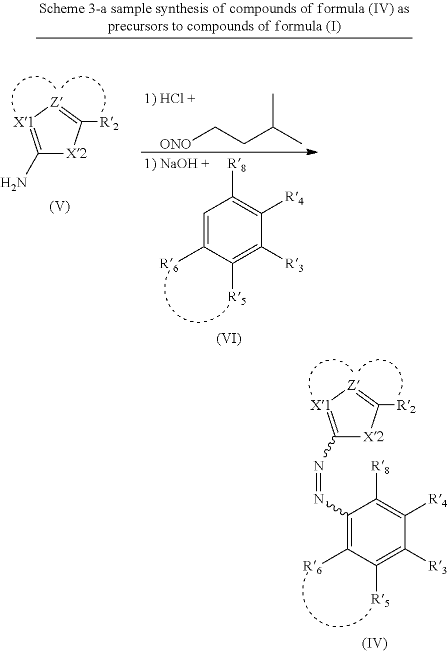

Compounds (IV) where R′₃═OH are often straightforward to form by this route. Therefore this synthesis is a preferred route towards compounds of structure (I) where R₃═OMe, as such compounds may straightforwardly be obtained from methylation of the precursor (IV) having R′₃═OH.

If R′₂, R′₃, R′₄, R′₅, R′₆, R′₈, X′1, X′2 and Z′ are, respectively, R₂, R₃, R₄, R₅, R₆, R₈, X1, X2 and Z, the compound (IV) directly corresponds to the desired compound (I). In other cases, the groups R′₂, R′₃, R′₄, R′₅, R′₆, R′₈, X′1, X′2 and/or Z′ are, respectively, to be converted to R₂, R₃, R₄, R₅, R₆, R₈, X1, X2 and/or Z with appropriate conditions and reactants, to obtain compound (I). This conversion can be carried out in one or several steps. Additionally, some compounds (I) can be obtained from another compound (I), especially in the case of prodrugs.

The reactions of compounds (II) with (III) and (V) with (VI) can be carried out according to adaptations of standard procedures^{[32,38,39]}. Other methods which may be appropriate for synthesising compounds (IV) as precursors to compounds (I), as well as other methods for synthesising different appropriate precursors to compounds (I), may also be adapted from the literature; mention may particularly be made of the Mills reaction, among other methods.^{[32,38,39]} The Mills reaction could be applied to couple (II) with (V) after one of these two aniline compounds were initially converted to its corresponding nitroso derivative. This aniline->nitroso conversion may be performed without requiring purification, so the Mills reaction can be used to conveniently form an azo compound^{[39]} which may be a compound (I) or else a precursor to a compound (I). An example of such a synthetic strategy using the Mills reaction is the synthesis of (I.30), given in the Examples.

The compounds (II), (III), (V) and (VI) can be commercially available or prepared with the use of classical or adapted chemical reactions.

Therefore, the molecules of formulae (I) can be prepared by diverse routes which are appropriate for tolerating different substitution patterns, using simple and well-understood chemistry, and can be obtained at a low preparative cost.

The salts of the compounds according to the invention are prepared according to well-known techniques to those skilled in the art. The salts of the compounds of formula (I) according to the present invention comprise those with inorganic or organic acids or bases which enable suitable separation or crystallization of the compounds of formula (I), and also pharmaceutically acceptable salts. As an appropriate acid, mention may be made of: oxalic acid or an optically active acid, for example a tartaric acid, a dibenzoyltartaric acid, a mandelic acid or a camphorsulfonic acid, and those which form physiologically acceptable salts, such as the hydrochloride, hydrobromide, sulfate, hydrogensulfate, dihydrogen phosphate, maleate, fumarate, 2-naphthalenesulfonate, para-toluenesulfonate, 2,2,2-trifluoroacetate, mesylate, besylate or isothionate salts. As an appropriate base, mention may be made of: lysine, arginine, meglumine, benethamine, benzathine and those which form physiologically acceptable salts, such as sodium, potassium or calcium salts.

As hydrated forms of compounds, mention may be made, by way of example, of hemihydrates, monohydrates and polyhydrates.

Compounds of formula (I) also comprise those in which one or more hydrogen, carbon or halogen atoms, in particular chlorine or fluorine atoms, have been replaced with their radioactive isotope, for example tritium or carbon-14. Such labelled compounds are of use in research, metabolism or pharmacokinetic studies, or in biochemical assays.

The functional groups optionally present in the compounds of formula (I) and in the reaction intermediates can be protected, either in a permanent form or in a temporary form, by protective groups which ensure unambiguous synthesis of the expected compounds. The protection and deprotection reactions are carried out according to techniques well known to those skilled in the art. The expressions “protective form” and especially “protective form for amines, alcohols, thiols or carboxylic acids” are intended to mean protective groups such as those described in Greene & Wuts^{[40]} or in Kocienski^{[41]}.

The compounds (I) according to the invention or their derivatives formed in vivo, in the case of prodrugs, are active, in a as form (I-cis), as tubulin polymerization inhibitors which are azoaryl isosteres of the combretastatin pharmacophore. Thus compounds (I) can be used in roles where combretastatins are appropriate, as well as in other applications, as eg. anti-mitotic, anti-angiogenic, antitumoral or chemotherapeutic agents; and in particular for the treatment of a cancer, such as melanoma, adenocarcinoma of the lung, neuroblastoma, small cell carcinoma of the lung, breast carcinoma, colon carcinoma, ovarian carcinoma, or bladder carcinoma; or for treatment of other diseases, especially those characterized by abnormal vascularisation, such as diabetic retinopathy, psoriasis, endometriosis, or rheumatoid arthritis or atherosclerosis. They may also be useful in fundamental research for precise, spatiotemporally-controllable and/or reversible inhibition of the tubulin cytoskeleton for diverse applications.

The compounds (I) according to the invention can be administered to a patient in need of such a treatment, in their as form (I-cis) or preferably in their trans form (I-trans) or as a mixture of the (I-cis) and (I-trans) isomers. They can be included in a pharmaceutical composition.

The compositions administrable to animals (including human beings) contain an effective dose of a compound according to the invention or of an acceptable salt, solvate or hydrate thereof, and at least a suitable excipient.

Said excipients are chosen according to the form and the mode of administration desired. In the pharmaceutical compositions of the present invention for oral, sublingual, subcutaneous, intramuscular, intravenous, topical, intratracheal, intranasal, transdermal, rectal or intraocular administration, the compound of formula (I), (I-trans) or (I-cis) above, or the optional salts, solvates and hydrates thereof, can be administered in unit administration forms, as a mixture with conventional pharmaceutical salts, to animals and to human beings for the prophylaxis or the treatment of diseases characterized by abnormal cellular proliferation (such as in cancers), abnormal vascularisation, and/or abnormal cellular migration. The appropriate unit administration forms include oral forms, such as tablets, gel capsules, powders, granules and oral solutions or suspensions, sublingual, buccal, intratracheal or intranasal administration forms, subcutaneous, intramuscular or intravenous administration forms and rectal administration forms. For topical application, the compounds according to the invention can be used in creams, ointments, lotions or eye lotions.

In order to obtain the effect, the dose of the compound of formula (I), (I-trans) or (I-cis) above, or the optional salts, solvates and hydrates thereof preferably ranges between 1 and 100 mg per kg of body weight and per day.

When a solid composition in tablet form is prepared, the main active ingredient is mixed with a pharmaceutical vehicle, such as gelatin, starch, lactose, magnesium stearate, talc, Arabic gum, or the like. The tablets can be coated with sucrose, with a cellulose-based derivative, or with other suitable materials, or else they can be treated such that they have a sustained or delayed activity and that they continuously release a predetermined amount of active ingredient.

A preparation in gel capsules is obtained by mixing the active ingredient with a diluent and by pouring the mixture obtained into soft or hard gel capsules. Pharmaceutical compositions containing a compound of the invention can also be in a liquid form, for example solutions, emulsions, suspensions or syrups. The appropriate liquid supports may be water, organic solvents such as glycerol or glycols, and also mixtures thereof, in varied proportions, in water. In preparation in syrup form, elixir form or for administration in the form of drops may contain the active ingredient together with a sweetener, preferably a calorie-free sweetener, methylparaben and propylparaben as antiseptic, and also a flavouring agent and a suitable colorant. The water-dispersible powders or granules can contain the active ingredient as a mixture with dispersants or wetting agents, or suspension agents, such as polyvinylpyrrolidone, and also with sweeteners or flavor enhancers.

Moreover, generally, the same preferences as those indicated previously for the compounds and compositions are applicable, mutatis mutandis, to the medicaments and uses employing these compounds. In the same way, the same preferences as those indicated previously for the compounds (I) in connection with the compositions, medicaments and uses employing these compounds are applicable, mutatis mutandis, to all the embodiments of compounds (A) and (B).

### EXAMPLES

The syntheses and descriptions of biological tests hereinafter, with reference to the appended schemes and figures, illustrate the invention without, however, limiting it.

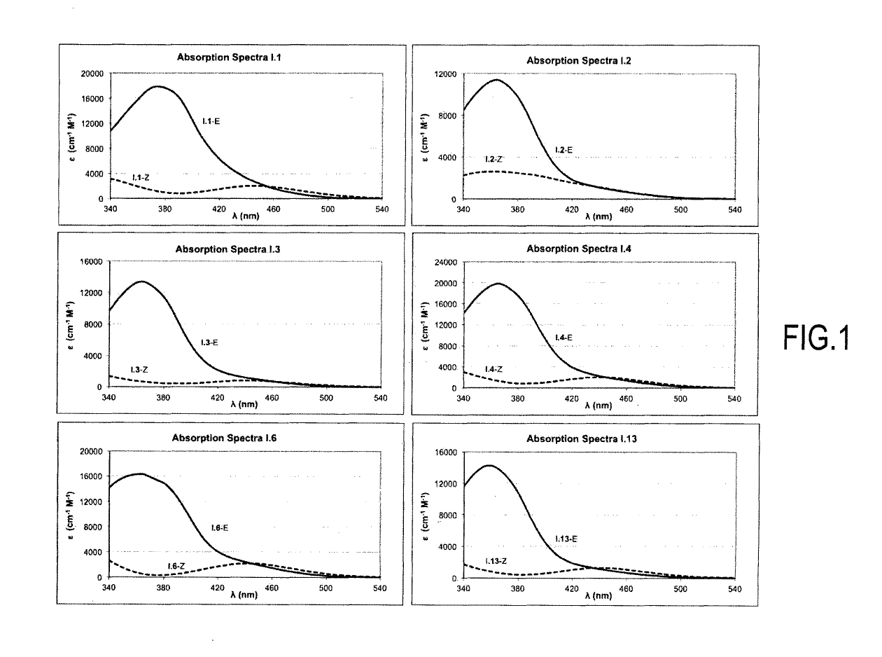

FIG. 1: Typical absorption spectra of trans (solid lines) and as (dotted lines) isomers of selected example compounds.

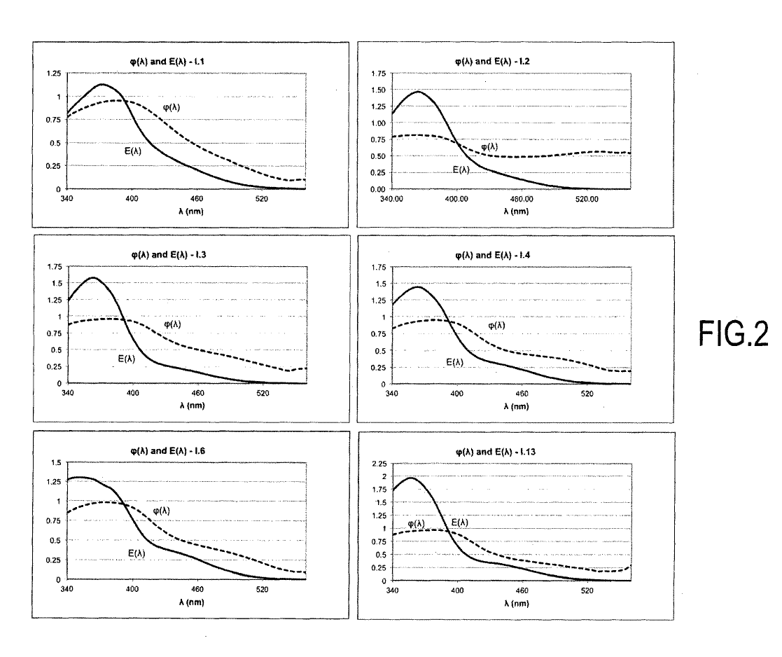

FIG. 2: typical φ(λ) (dotted lines) and E(λ) (solid lines) of selected example compounds.

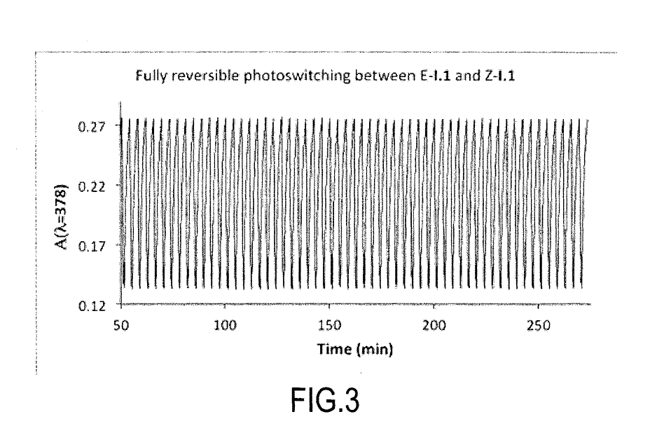

FIG. 3: Raw absorbance values A(λ_{strong}=378 nm) of a sample of I.1 (18 μM in non-degassed PBS left open to the atmosphere, containing 10% MeCN, at 37° C.) measured over time, while being reversibly photoisomerised between majority-cis and majority-trans forms by alternating the irradiating wavelength between λ₁=388 nm (50 s; bulk trans->cis) and λ₂=508 nm (180 s, bulk cis->trans). Higher absorbance corresponds to a greater amount of trans isomer.

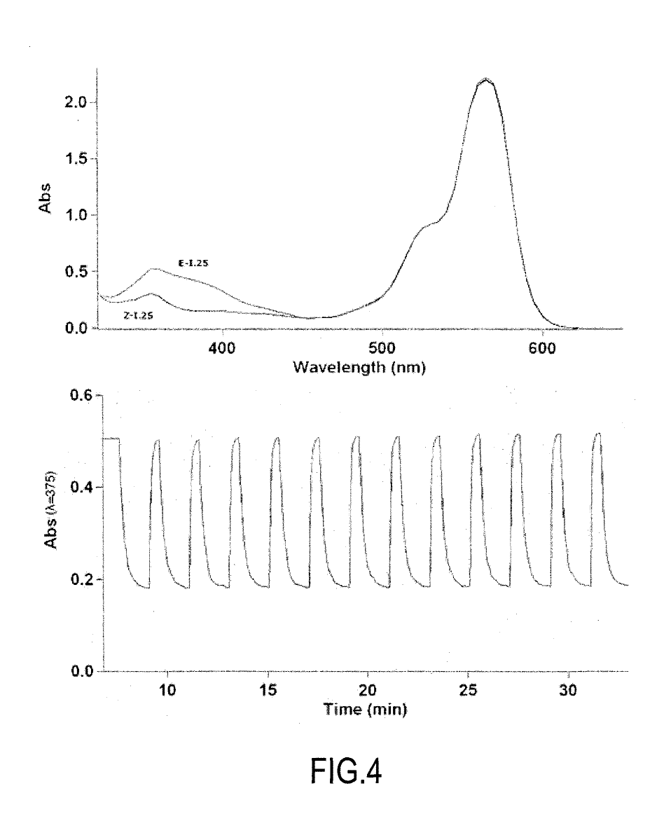

FIG. 4: Raw data from UV-Vis measurements of the absorbance of a sample of compound I.25 at 33 μM in PBS containing 20% MeCN at 37° C. Top panel: absorbance spectra of the same sample photoisomerised to contain either 100%-trans (spectrum “E-I.25”, generated by irradiation at 554 nm) or a majority of as (spectrum “Z-I.25”, >3:1 ratio of cis:trans isomers, generated by irradiation at 384 nm). Bottom panel: A(λ_{strong}=375 nm) measured while the irradiating wavelength was held alternately at λ₁=384 nm (140 s; bulk trans->cis, decrease of absorption) then λ₂=554 nm (40 s, quantitative cis->trans; return of absorption to 0.51). Note that at 6 minutes, the sample is in the dark-adapted (all-trans) state, as irradiations have not yet commenced.

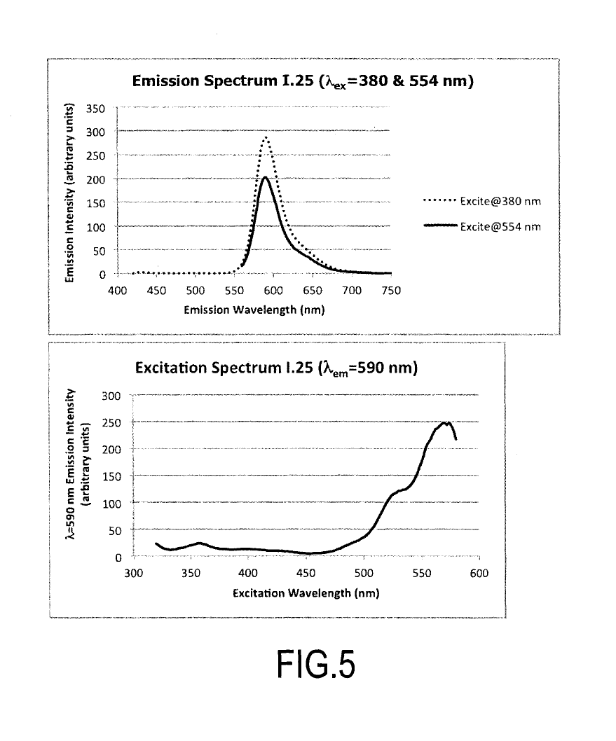

FIG. 5: Fluorescence spectrophotometry measurements of a sample of compound I.25 at 10 μM in 60:32:8 EtOH:PBS:MeCN at 25° C. Top panel: emission spectra showing that I.25 can be excited at either 380 nm (dotted line) or 554 nm (solid line) to produce fluorescence with an emission maximum at 590 nm (note: vertical scale is in arbitrary units not comparable between measurements). Bottom panel: excitation spectrum showing that I.25 can be excited over a range of wavelengths to produce fluorescence at 590 nm.

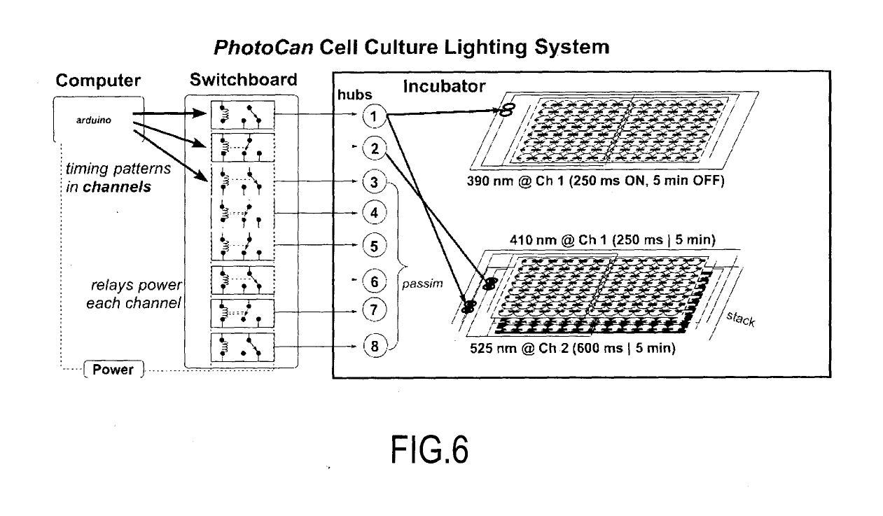

FIG. 6: Schematic presentation of a computer-controlled LED-based automatic lighting system for cell culture experiments. The system was designed to evaluate potential in vivo medicinal uses of the compounds of the invention in an in vitro cell culture model. For example, both toxic regimes (eg. 390 nm irradiation for 250 ms pulsed every 5 min) and strong rescue regimes (eg. 410 nm irradiation for 250 ms then 525 nm irradiation for 600 ms synchronously pulsed every 5 min) could easily and cheaply be applied in parallel to many standard multiwell cell culture plates, during incubations maintained over several days.

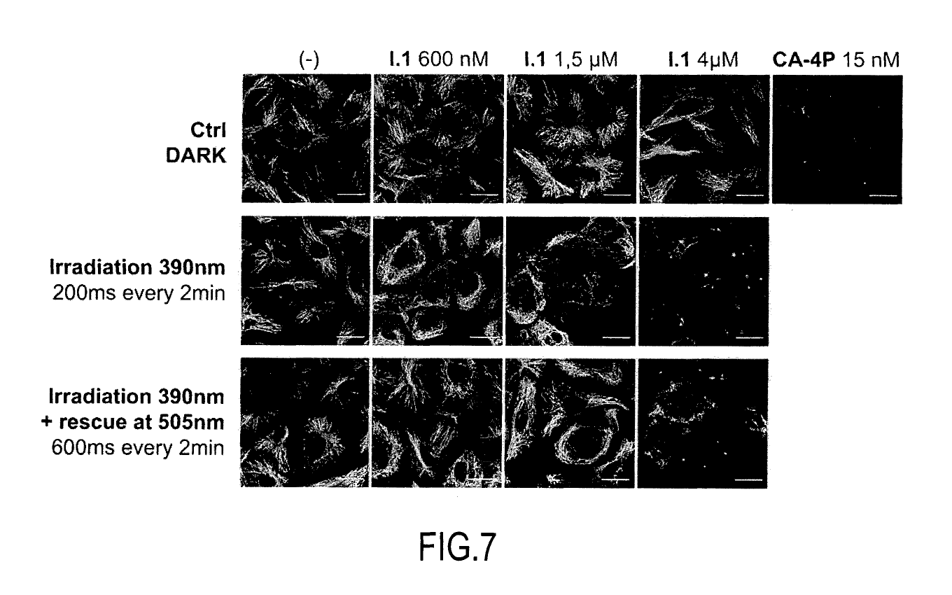

FIG. 7: Immunofluorescence microscopy staining images showing in cellulo light-controlled effects of compound I.1 on the structure of microtubules. MDA-MB-231 cells were treated for 20 h with the indicated concentrations of compound I.1 and kept in the dark, or exposed to the 390 nm protocol (200 ms every 2 min), or exposed to the double irradiation rescue protocol (200 ms of 390 nm, then immediately 600 ms of 505 nm, every 2 min). Representative confocal microscope images are shown. White scale bars in the lower right of each panel correspond to a scale of 20 μm.

### EXAMPLES PART A

### Chemical Synthesis

### Reagents and Procedures:

Unless stated otherwise, (1) all reactions and characterisations were performed with unpurified, undried, non-degassed solvents and reagents, used as obtained, under closed air atmosphere without special precautions; (2) “hexane” used for chromatography was distilled from commercial crude isohexane fraction on rotavap; (3) “column” and “chromatography” refer to flash column chromatography, which was performed on Merck silica gel Si-60 (40-63 μm); (4) procedures and yields are unoptimized; (5) yields refer to isolated chromatographically and spectroscopically pure materials, corrected for residual solvent content; (6) all eluent and solvent mixtures are given as volume ratios unless otherwise specified, thus “1:1 Cy:EA” indicates a 1:1 mixture of cyclohexane and ethyl acetate by volume.

Thin-layer chromatography (TLC) was run on 0.25 mm Merck silica gel plates (60, F-254). UV light (254 nm) was used as a visualising agent, and standard TLC dips based on p-anisaldehyde (Anis), Hanessian's cerium ammonium molybdate formulation (Han), 0.6% methanolic FeCl₃ (FeCl₃), basic KMnO₄ (KMnO₄), phosphomolybdic acid (PMA), Dragendorff's reagent (Drag), vanillin (Van) and ninhydrin (Nin) followed by heating where necessary were used as developing agents. R_{f} values were usually determined in hexane:ethyl acetate (Hx:EA) or cyclohexane:ethyl acetate (Cy:EA) eluents, reported as volume ratio compositions (v:v). TLC characterisations are thus abbreviated as per (R_{f}=0.09 on 6:1 Hx:EA, Anis).

### NMR:

Standard NMR characterisation was by ¹H and ¹³C 1D-NMR spectra. Known compounds were checked against literature data and their spectral analysis is not detailed unless necessary. Spectrometers used were Bruker DPX 200 (200 MHz & 50 MHz for ¹H and ¹³C respectively), Bruker Ascend 300 (300 MHz, 75 MHz and 282 MHz for ¹H, ¹³C and ¹⁹F respectively), Bruker Ascend 400 (400 MHz & 100 MHz for ¹H and ¹³C respectively), Bruker AVANCE 500 (500 MHz & 125 MHz for ¹H and ¹³C respectively), as indicated, at 300K. Where not indicated otherwise, the NMR solvent was CDCl₃. Chemical shifts (δ) are reported in ppm calibrated to residual non-perdeuterated solvent as an internal reference.^{[42]} The following peak descriptions are used: singlet (s), doublet (d), triplet (t), quartet (q), multiplet (m), broad (br), quintet (quin), sextet (sext); apparent multiplicities (resolved by 2D experiments or determined by complete spectral assignment) are denoted by a tilde, eg. “doublet of doublets, appears as a triplet with apparent coupling constant J=3 Hz” is denoted (˜t, 3 Hz).

### Mass Spectra:

Unit mass measurements were performed on AGILENT 1100 SL and AGILENT 1200 SL coupled LC-MS systems with ESI mode ionisation, with binary eluent mixtures of water-acetonitrile, with the water containing sodium/ammonium formate or formic acid. Both direct injection of the sample (abbreviated DIMS) and LCMS were performed as specified. For LCMS, unless stated otherwise, an Eclipse Plus 3.5 μm/4.6×100 mm C18 column was used at 25° C. with a 2 mL/min flow rate such that the sample solvent front eluted at tᵣₑₜ=0.76 min. A linear gradient of eluent composition from 90:10->10:90 water:acetonitrile was applied over the first 4.5 min, then 10:90 maintained until all peaks of interest had been observed (typically a further 3 min). Ion peaks from (positive/negative mode) are reported as (+/−) with units Th (m/z), thus ‘DIMS(+): 328 @ 100, 227 @ 80’ indicates ESI with direct injection giving two positive ion peaks at m/z=328 and 227 Th, with the peak at 227 Th being 80% of the height of the peak at 328 Th (isotopic peak patterns were sometimes useful to confirm molecular identity on DIMS spectra). Unless stated otherwise, all reported peaks in the positive mode were [MH]⁺ peaks, and all observed peaks in the negative mode were [M-H]⁻ peaks. HRMS was carried out by the Service Central d'Analyse du CNRS, Solaize, France, and by the Zentrale Analytik of the LMU, Munich using ESI or EI ionisation as specified.

### Other Information:

The following abbreviations are used: Hx—distilled isohexanes, Cy—cyclohexane, EA—ethyl acetate, peth—petroleum ether 40-60° fraction, DCM—dichloromethane, TFA—2,2,2-trifluoroacetic acid, iPenONO—isopentyl nitrite, PBS—phosphate buffer saline, HOBt—1-hydroxybenzotriazole, DCC—dicyclohexylcarbodiimide, DMF—dimethylformamide, brsm—based on recovered starting material, Ts or tosyl—para-toluenesulfonyl, Boc—tert-butoxycarbonyl, Ser—L-serinyl, Leu—L-leucyl, TBS—tert-butyldimethylsilyl, Et—ethyl, Ac—acetyl, Me—methyl, MeCN—acetonitrile, iPr—isopropyl, iPen—iso-pentyl, Bu—butyl, DMAP—4-(dimethylamino)pyridine, DBU—1,8-diazabicycloundec-7-ene, DMAP—4-dimethylaminopyridine, RBpip—N-(6-(diethylamino)-9-(2-(piperazine-1-carbonyl)phenyl)-3H-xanthen-3-ylidene)-N-ethylethanaminium, wt % —percentage by weight. Where Standard Procedures were used in synthesis, the amounts of reactants, reagents and solvents employed were implicitly adjusted to maintain the same molar ratios as in the given Procedure, and no other alterations from the Standard Procedure (eg reaction time, choice of extraction solvent, temperature) were made, unless stated otherwise. In these synthetic descriptions in Part A, azobenzenes are drawn by default in their cis-isomeric form to enable easier comparison with the SAR literature of their isosteric antitubulin stilbenes and stilbenoid compounds such as the combretastatins. However, this should be understood to imply either or both of the trans & as forms constituting a given sample depending on light exposure, therefore they are also named without E/Z-designations.

### Standard Procedure A: Diazo Coupling Using Isopentyl Nitrite

To the aniline (1 mmol) were added MeOH (5 mL) and conc. HCl (0.25 mL), and the mixture cooled in an icebath. A solution of iso-pentyl nitrite (1.02 mmol) in methanol (0.6 mL) was added dropwise and the reaction stirred for 30 min in the cold. A cold solution of the phenol (1.05 mmol) in methanol (2 mL) and NaOH (2.0 M, 1.8 mL) was prepared, and to it was added the solution of the diazonium, dropwise over 1 minute. After typically 30 minutes stirring in the cold, the pH was adjusted to 7 with phosphate buffer, chloroform (10 mL) was added, and the aqueous phase was extracted with CHCl₃ (2×10 mL). The combined organic layers were washed with water (15 mL) and brine (10 mL), dried on Na₂SO₄, filtered and concentrated. Chromatography with a Hx:EA gradient was used to separate the para-phenolic azobenzene product which typically ran as a single isomer during chromatography.

### Standard Procedure B: Phenol Methylation in Acetone

To the phenol (1 mmol) were added K₂CO₃ (3 mmol), technical grade acetone (10 mL), and MeI (2 mmol), and the mixture stirred at RT for 2-12 h, until TLC showed satisfactory conversion of the starting material. Note that TLC often separated the trans and cis azobenzene isomers, with the major spot apparently being the faster-running trans isomer; the as isomer typically appeared at near-identical R_{f} to that of the starting phenol. The volatiles were evaporated on the rotavap, then the crude mixture was separated by chromatography with a Hx:EA gradient. Since the para-O-methylated trans and as product isomers typically were separable by chromatography, the crude product could optionally be kept in the dark overnight and protected from light during loading and chromatography (eg wrapping the column with aluminium foil) to ensure cleaner separation of the desired product (as the trans isomer) from other impurities, though this was typically not necessary.

Standard Procedure C: Phenol Methylation Using MeI and Ag₂CO₃ in Toluene

To the phenol (1 mmol) in a screw-cap pressure tube were added toluene (6 mL), Ag₂CO₃ (1 mmol, supported on Celite or not), and MeI (1.5 mmol). The tube was sealed, protected from light, and the reaction heated to 110° C. overnight with stirring. After cooling, the crude reaction mixture was filtered, the residue washed with chloroform (2 mL), and the combined filtrates concentrated and separated on column as for Standard Procedure B.

Azocombretastatin A-4 (I.1) and Methylated Derivative (I.11)

The synthesis of I.1 and I.11 is presented on Scheme 4 hereafter.

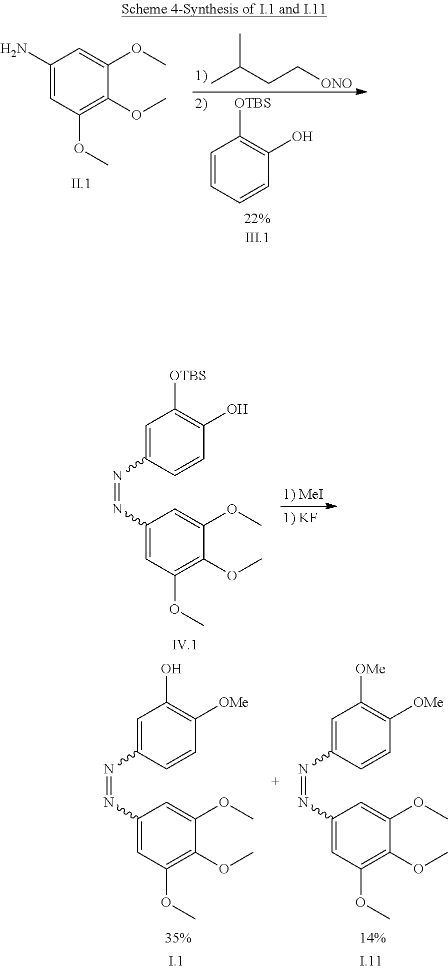

### 2-((tert-butyldimethylsilyl)oxy)phenol (III.1)

Catechol (580 mg, 5.27 mmol) was added to a stirred solution of TBSCl (658 mg, 4.4 mmol) and imidazole (850 mg, 11.6 mmol) in DMF (15 mL), then NEt₃ (1 mL, 7.5 mmol) was added and a precipitate formed. The reaction mixture was stirred overnight, concentrated on the rotavap, and partitioned between water (75 mL) and ethyl acetate (25 mL). The aqueous phase was extracted twice with ethyl acetate (2×25 mL), then the combined organic extracts were washed with water (2×25 mL), brine (10 mL), dried on Na₂SO₄, filtered and evaporated to yield a pale yellow crude (980 mg) of which 817 mg was purified by chromatography on 100:0->20:1->10:1 Hx:EA giving III.1 as colourless oil (567 mg, 75%; R_{f}=0.56 on 9:1 Hx:EA, Han). ¹H-NMR matched literature data^{[43]}.

### 2-((tert-butyldimethylsilyl)oxy)-4-((3,4,5-trimethoxyphenyl)diazenyl)phenol (IV.1)

By Standard Procedure A, commercial 3,4,5-trimethoxyaniline (II.1; 236 mg, 1.29 mmol) was reacted with III.1 (250 mg, 1.12 mmol) to yield a deep red crude oil. Chromatography on 5:1->1:1 Hx:EA returned IV.1 as a yellow oil (102 mg, 0.244 mmol, 22%; R_{f}=0.24 on 5:1 Hx:EA). NMR of the product as isolated ex organic solvent revealed a roughly 55:44 proportion of [presumably trans and cis] isomers when analysed in CDCl₃ without precautions to block ambient light. Their spectra could for some peaks be separated (denoted _{E} or _{Z}): ¹H-NMR (400 MHz): δ=7.57 (˜td, Hz, 1H), 7.47-7.45 (m, 1H), 7.23 (s, 2H_{Z}) & 7.21 (s, 2H_{E}), 7.07 (d, 8.5 Hz, 1H_{E}) & 6.97 (d, 8.5 Hz, 1H_{Z}), 5.88 (s br, 1H_{E}) & 5.66 (s br, 1H_{Z}), 3.98 (s, 6H_{E}) & 3.97 (5, 6 Hz), 3.94 (s, 3H), 1.06 (s, 9H_{E}) & 1.05 (s, 9H_{Z}), 0.36 (s, 6H_{E}) & 0.34 (s, 6H_{Z}) ppm. ¹³C-NMR (100 MHz): δ=153.5 (×2, E & Z), 150.3 & 148.6 (E & Z), 148.5 & 142.9 (E & Z), 147.9 (E & Z), 146.5 & 145.4 (E & Z), 140.2 & 140.1 (E & Z), 119.6 & 118.1 (E & Z), 117.5 & 114.6 (Z & E), 110.7 & 106.9 (E & Z), 100.2 & 100.1 (×2, E & Z), 61.0 (E & Z), 56.2 & 56.2 (×2, E & Z), 25.8 & 25.7 (×3, E & Z), 18.3 & 18.2 (E & Z), −4.2 & −4.3 (×3, E & Z) ppm. LCMS(+): tᵣₑₜ=5.6 & 5.8 min, each 419 Th═[MH]⁺: these peaks were assigned to the cis & trans isomers respectively since the UV absorption profile of the first peak showed a secondary absorption band between 450-510 nm (cis), while the second peak was generally more intense especially between 320-380 nm (trans) but without this secondary band. HRMS (ESI+) calcd for [C₂₁H₃₃N₂O₆Si]⁺═[M.H₃O⁺]: m/z 437.210. found 437.236.

### 2-methoxy-5-((3,4,5-trimethoxyphenyl)diazenyl)phenol, “Azocombretastatin A-4” (I.1) and 1-(3,4-dimethoxyphenyl)-2-(3,4,5-trimethoxyphenyl)diazene (I.11): procedure 1

To IV.1 (73 mg, 0.175 mmol) were added K₂CO₃ (44 mg, 0.32 mmol), dry DMF (2 mL), and MeI solution (1.37 g of a 4.3 wt % solution in DMF, 0.43 mmol), and the mixture stirred at RT for 2 h until TLC (5:1 Hx:EA) showed complete conversion of the starting material to a faster-running product. The volatiles were evaporated at 60° C. and 5 mbar, then THF (8 mL) and an aqueous solution of KF (1 M, 5 mL) were added to the residue and the mixture stirred at RT for 3 h 30 min. The bulk of the THF was removed on the rotavap, then water (15 mL), brine (2 mL), and KH₂PO₄/K₂HPO₄ buffer (2 M, pH=6.8, 4 mL) were added and the aqueous phase extracted with dichloromethane (3×15 mL). The combined organic layers were washed with water (15 mL) and brine (10 mL) and dried on Na₂SO₄, filtered and concentrated to a crude oil. Flash chromatography with a very gentle gradient covering 5:1->2.4:1 Hx:EA in the dark (aluminium foil wrapped around the column) separated the crude components cleanly without problems due to the different R_{f} values of their trans and cis isomers, returning I.11 (8.0 mg, 0.024 mmol, 14% over 2 steps) then I.1 (19.7 mg, 0.062 mmol, 35% over 2 steps).

### I.1:

R_{f} (trans/cis)=0.36 and 0.18 on 1.7:1 Hx:EA (Anis); orange solid. NMR of the product as isolated with precautions to block ambient light revealed a single geometric isomer. ¹H-NMR (400 MHz): δ=7.59-7.53 (m, 1H), 7.24 (s, 2H), 7.00 (d, 8.4 Hz, 1H), 5.84-5.66 (s br, 1H), 4.01 (s, 3H), 3.99 (s, 6H), 3.95 (s, 3H) ppm. ¹³C-NMR (100 MHz): δ=153.5 (×2), 149.2, 148.5, 147.3, 146.2, 140.2, 119.0, 110.1, 106.0, 100.2 (×2), 61.0, 56.2 (×2), 56.2 ppm. LCMS(+): tᵣₑₜ=3.12 & 3.89 min, each 319 Th═[MH]⁺: these peaks were assigned to the cis & trans isomers respectively since the first peak had a secondary absorption band at 445 nm. HRMS (EI+) calcd for [C₁₆H₁₃N₂O₅]⁺═[M⁺]: m/z 318.1288. found 318.1287.

### I.11:

R_{f} (trans/cis)=0.34 and 0.14 on 2.4:1 Hx:EA; R_{f} (trans/cis)=0.53 and 0.28 on 1.7:1 Hx:EA (Anis); yellow solid. ¹H-NMR (400 MHz, CD₃CN): δ=7.60 (dd, 8.5&2.2 Hz, 1H), 7.52 (d, 2.2 Hz, 1H), 7.26 (s, 2H), 7.12 (d, 8.5 Hz, 1H), 3.93 (s, 6H), 3.93 (s, 3H), 3.91 (s, 3H), 3.83 (s, 3H) ppm. ¹³C-NMR (100 MHz, CD₃CN): δ=154.3 (×2), 152.8, 150.4, 149.1, 147.1, 140.7, 120.9, 111.6, 102.4, 100.5 (×2), 60.6, 56.3 (×2), 56.2, 55.9 ppm. LCMS(+): tᵣₑₜ=3.45 & 4.35 min, each 333.1 Th═[MH]⁺: these peaks were assigned to the cis & trans isomers respectively since the first peak had a secondary absorption band at 440 nm. HRMS (ESI+) calcd for [C₁₇H₂₁N₂O₅]⁺═[MH⁺]: m/z 333.1445. found 333.1443.

### (I.1) and (I.11): Procedure 2:

Alternatively, by Standard Procedure B, IV.1 (100 mg, 0.23 mmol) was methylated in acetone (5 mL) using MeI (80 mg, 0.56 mmol) and K₂CO₃ (420 mg, 3 mmol). After evaporation of the volatiles, the residue was partitioned between CHCl₃ (10 mL) and aqueous phosphate buffer (pH=3), extracted with CHCl₃ (2×10 mL), and pad filtered on silica using 2.4:1 Hx:EA eluent, yielding 94 mg crude red oil. To this under nitrogen atmosphere were added MeCN (6 mL) and HF (70% in pyridine, 165 mg), and the reaction stirred for 15 minutes. CaCO₃ (1.0 g), CaCl₂ (0.5 g) and water (10 mL) were added to quench excess HF, the pH adjusted to 3 with KH₂PO₄, then the mixture was extracted with CHCl₃ (3×10 mL). The combined organic layers were washed with brine (10 mL), dried on Na₂SO₄, filtered and concentrated to a black crude powder (70 mg). Flash chromatography with 5:1->2.4:1 Hx:EA in the dark (aluminium foil wrapped around the column) separated the crude components cleanly without problems due to the different R_{f} values of their trans and as isomers, giving I.11 (5 mg, 0.015 mmol, 6%) and I.1 (14 mg, 0.044 mmol, 19% over 2 steps), both spectroscopically identical to the products of procedure 1.

### Azocombretastatin A-4 (I.1), Alternative Procedure

An alternative synthesis giving I.1 without generating I.11, using tosyl instead of TBS as a protecting group, is presented on Scheme 5 hereafter.

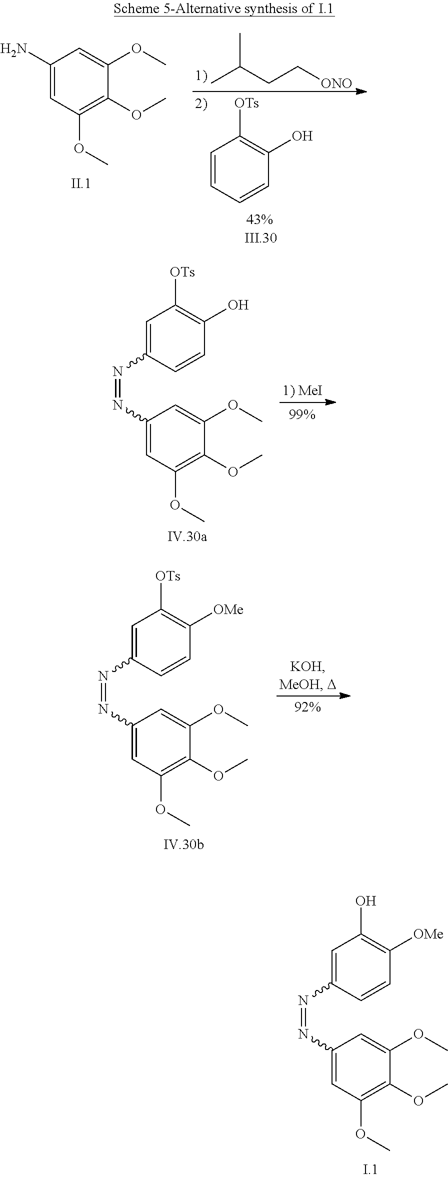

### 2-hydroxy-5-((3,4,5-trimethoxyphenyl)diazenyl)phenyl 4-methylbenzenesulfonate (IV.30a)

Commercial 3,4,5-trimethoxyaniline (II.1; 1.045 g, 5.71 mmol) was reacted with known 2-hydroxyphenyl 4-para-toluenesulfonate^{[44]} (1.508 g, 5.71 mmol) by Standard Procedure A except that stirring of the mixture of phenolate and diazonium was continued for 5 h at 0° C. to allow for more complete conversion. Following workup, the deep red crude oil was chromatographed on 5:1->1:1 Hx:EA returning IV.30a as a yellow oil (1.130 g, 2.47 mmol, 43%; R_{f}=0.43 on 1:1 Hx:EA). ¹H-NMR (400 MHz): δ=7.76 (d, 8.4 Hz, 2H), 7.72 (dd, 8.7 & 2.3 Hz, 1H), 7.51 (d, 2.3 Hz, 1H), 7.29 (dd, 8.5 & 0.8 Hz, 2H), 7.11 (s, 2H), 7.04 (d, 8.7 Hz, 1H), 3.89 (s, 6H), 3.86 (s, 3H), 2.39 (s, 3H) ppm. ¹³C-NMR (100 MHz): δ=153.5 (×2), 150.7, 148.1, 146.4, 146.4, 140.7, 137.3, 131.2, 130.1 (×2), 128.7 (×2), 124.0, 118.1, 117.4, 100.4 (×2), 61.1, 56.2 (×2), 21.8 ppm. LCMS(+): tᵣₑₜ=4.60 min, 459 Th═[MH]⁺. HRMS (ESI+) calcd for [C₂₂H₂₃N₂O₇S]⁺═[MH⁺]: m/z 459.12205. found 459.12168.

### 2-methoxy-5-((3,4,5-trimethoxyphenyl)diazenyl)phenyl 4-methylbenzenesulfonate (IV.30b)

By Standard Procedure B, IV.30a (700 mg, 1.53 mmol) was methylated overnight. Chromatography of the red crude solid on 5:1->1:1 Hx:EA returned IV.30b (712 mg, 1.51 mmol, 99%; R_{f}=0.62 and 0.46 on 1:1 Hx:EA, FeCl₃) as a red oil. ¹H-NMR (400 MHz, DMSO): δ=7.93 (dd, 8.8 & 2.4 Hz, 1H), 7.75 (d, 8.4 Hz, 2H), 7.63 (d, 2.4 Hz, 1H), 7.49 (d, 8.5 Hz, 2H), 7.28 (d, 8.9 Hz, 1H), 7.23 (s, 2H), 3.91 (s, 6H), 3.77 (s, 3H), 3.61 (s, 3H), 2.44 (s, 3H) ppm. ¹³C-NMR (100 MHz, DMSO): δ=154.2, 153.8 (×2), 148.0, 146.3, 145.6, 140.7, 138.5, 132.3, 130.4 (×2), 128.8 (×2), 126.2, 115.6, 113.9, 100.7 (×2), 60.7, 56.6, 56.5 (×2), 21.6 ppm. LCMS(+): tᵣₑₜ=4.48 & 5.17 min, 473 Th═[MH]⁺: these peaks were assigned to the cis & trans isomers respectively since the UV absorption profile of the first peak (cis) featured a shoulder centred around 450 nm. HRMS (ESI+) calcd for [C₂₃H₂₅N₂O₇S]⁺═[MH⁺]: m/z 473.13770. found 473.13730.

### 2-methoxy-5-((3,4,5-trimethoxyphenyl)diazenyl)phenol, “Azocombretastatin A-4” (I.1)

To IV.30b (525 mg, 1.10 mmol) were added KOH (1.25 g) and MeOH (25 mL) and the solution heated to 80° C. for 1 hour. After evaporation of the volatiles, the residue was partitioned between EtOAc (20 mL) and aqueous KH₂PO₄ solution (10%, 30 mL), then the aqueous layer was extracted with EtOAc (2×10 mL). The combined organic layers were washed with water (20 mL), brine (10 mL), dried on Na₂SO₄, filtered and concentrated. The crude oil was chromatographed on 5:1->1:1 Hx:EA, giving I.1 (320 mg, 1.01 mmol, 92%) as an orange solid, identical by NMR and LCMS to that synthesised previously from IV.1b (shown above).

### North Ring Meta-Amino Derivative (I.2)

The synthesis of I.2 is presented on Scheme 6 hereafter.

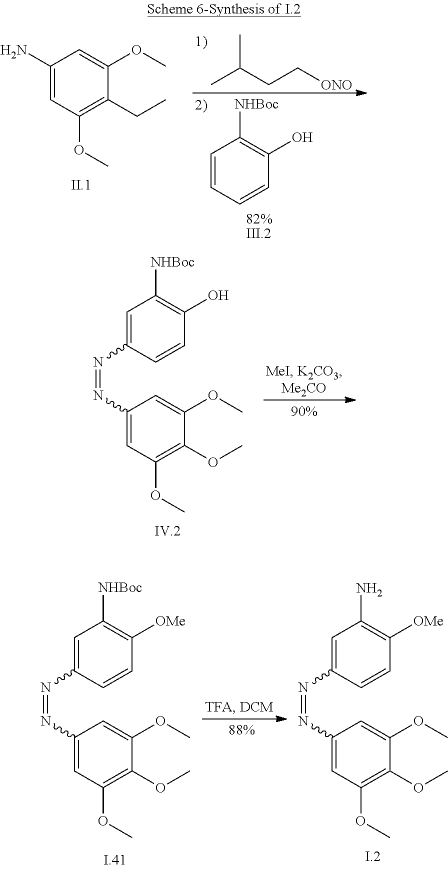

### tert-butyl (2-hydroxyphenyl)carbamate (III.2)

2-aminophenol (3.93 g, 36 mmol) was stirred with tert-butoxycarbonyl dicarbonate (8.32 g, 38 mmol) in dry pyridine (30 mL) with triethylamine (4 mL) warming from 0° C. to 25° C. over 12 h. The volatiles were evaporated and the residue partitioned between diethyl ether and phosphate buffer (pH=10); the ether layer was washed with phosphate buffer then brine, dried on Na₂SO₄, filtered and evaporated to yield 8.1 g of dark crude product which could be purified by column chromatography (20:1->5:1 Hex:EA), or by fractional crystallisations from acetone-hexane followed by hot hexane trituration. NMR spectra matched literature data^{[45]}: ¹H-NMR (400 MHz): δ=8.15 (s br, 1H), 7.08-7.00 (m, 2H), 6.99 (d, 7.9 Hz, 1H), 6.88 (˜t, 7.5 Hz, 1H), 6.69 (s, 1H), 1.56 (s, 9H) ppm. ¹³C-NMR (100 MHz): δ=155.1, 147.6, 125.7, 125.5, 121.5, 120.7, 119.1, 82.2, 28.3 (×3) ppm. DIMS(+): 210 Th=[MH]⁺.

### tert-butyl (2-hydroxy-5-((3,4,5-trimethoxyphenyl)diazenyl)phenyl)carbamate (IV.2)

By Standard Procedure A, II.1 (368 mg, 2.01 mmol) was reacted with III.2 (406 mg, 1.94 mmol). Chromatography on 5:1->2.4:1 Hx:EA returned IV.2 (642 mg, 1.59 mmol, 82%; R_{f}=0.22 on 2.4:1 Hx:EA, FeCl₃) as a brown viscous oil. ¹H-NMR (400 MHz, CD₃CN): δ=8.36 (s, 1H), 8.32 (d, 2.3 Hz, 1H), 7.54 (dd, 8.5 & 2.4 Hz, 1H), 7.35 (s, 1H), 7.19 (s, 2H), 7.02 (d, 8.5 Hz, 1H), 3.89 (s, 6H), 3.80 (s, 3H) ppm. ¹³C-NMR (100 MHz, CD₃CN): δ=153.7 (×2), 153.5, 149.0, 148.5, 146.1, 140.1, 127.5, 120.6, 115.4, 112.3, 100.0 (×2), 80.6, 60.0, 55.8 (×2), 27.52 (×3) ppm. LCMS(+): tᵣₑₜ=4.65 min, 404 Th═[MH]⁺. HRMS (ESI+) calcd for [C₂₀H₂₆N₃O₆]⁺═[MH⁺]: m/z 404.1816. found 404.1817.

### tert-butyl (2-methoxy-5-((3,4,5-trimethoxyphenyl)diazenyl)phenyl)carbamate (I.41)

By Standard Procedure B, IV.2 (637 mg, 1.58 mmol) was methylated overnight with MeI (448 mg, 3.13 mmol) and K₂CO₃ (873 mg, 6.32 mmol). Chromatography of the black crude solid on 5:1->2.4:1 Hx:EA returned I.41 (593 mg, 1.42 mmol, 90%; R_{f}=0.41 on 2.4:1 Hx:EA, FeCl₃) as a red oil. ¹H-NMR (400 MHz): δ=8.64 (s br, 1H), 7.58 (dd, 8.7, 2.4 Hz, 1H), 7.20 (s, 2H), 7.09 (s, 1H), 6.90 (d, 8.7 Hz, 1H), 3.90 (s, 9H), 3.86 (s, 3H), 1.49 (s, 9H) ppm. ¹³C-NMR (100 MHz): δ=153.4 (×2), 152.6, 149.8, 148.4, 146.7, 140.2, 128.8, 119.1, 111.2, 109.6, 100.4 (×2), 80.7, 61.0, 56.2 (×2), 56.0, 28.4 (×3) ppm. LCMS(+): tᵣₑₜ=4.57 & 5.42 min, 418 Th═[MH]⁺: these peaks were assigned to the cis & trans isomers respectively since the UV absorption profile of the first peak (cis) featured a shoulder centred around 440 nm. HRMS (ESI+) calcd for [C₂₁H₂₈N₃O₆]⁺═[MH⁺]: m/z 418.19726. found 418.19718.

### 2-methoxy-5-((3,4,5-trimethoxyphenyl)diazenyl)aniline (I.2)

To I.41 (590 mg, 1.41 mmol) were added CH₂Cl₂ (6 mL) and CF₃COOH (5 mL) and the purple solution stirred overnight at room temperature. The volatiles were removed under high vacuum, the residual TFA neutralised with addition of CHCl₃ (10 mL) and K₂CO₃ (618 mg) and the residue chromatographed on 1:1:0->1:1:1 Hx:EA:MeOH, giving I.2 as a green-black powder (394 mg, 1.24 mmol, 88%; R_{f}=0.56 on 1:1 Hx:EA (Van)). NMR when analysed in CDCl₃ without precautions to block ambient light showed two isomers in approximately 2:1 ratio [presumably trans and cis forms, attributed by HSQC, denoted _{E} and _{Z}]. ¹H-NMR (400 MHz): δ=8.84 (s br, ‘1H’, NH₂), 7.72 (dd, 8.5 & 1.9 Hz, 1H_{E}), 7.58 (d, 2.2 Hz, 1H_{Z}), 7.56 (d, 1.9 Hz, 1H_{E}), 7.53 (dd, 8.6 & 2.4 Hz, 1H_{Z}), 7.26 (d, 8.5 Hz, 1H_{E}), 7.19 (s, 2H_{E}), 7.16 (s, 2H_{Z}), 6.90 (d, 8.6 Hz, 1H_{Z}), 3.92-3.84 (m, 12H_{E} & 12H_{Z}) ppm. ¹³C-NMR (100 MHz): δ=153.6 & 153.5 (×2, E&Z), 151.6 & 149.4 (×1, E&Z), 148.5 & 148.1 (×1, E&Z), 146.8 & 145.7 (×1, E&Z), 140.8 & 140.2 (×1, E&Z), 130.0 (×1, E&Z), 121.7 & 120.7 (×1, E&Z), 110.4 & 110.3 (×1, E&Z), 110.1 & 101.3 (×1, E&Z), 100.5 & 100.2 (×2, E&Z), 61.1 & 61.0 (×1, E&Z), 56.2 & 56.2 (×2, E&Z), 56.1 (×1, E&Z) ppm. LCMS(+): tᵣₑₜ=3.04 & 3.94 min, each 318 Th═[MH]⁺: these peaks were assigned to the cis & trans isomers respectively since the first peak featured an absorption shoulder at 450 nm which was absent in the second peak (trans). HRMS (ESI+) calcd for [C₁₆H₂₀N₃O₄]⁺═[MH⁺]: m/z 318.1448. found 318.1449.

### North Ring Ortho-Oxy, Meta-Hydro Derivatives (I.8) and (I.12)

The syntheses of I.8 and I.12 are presented on Scheme 7 hereafter.

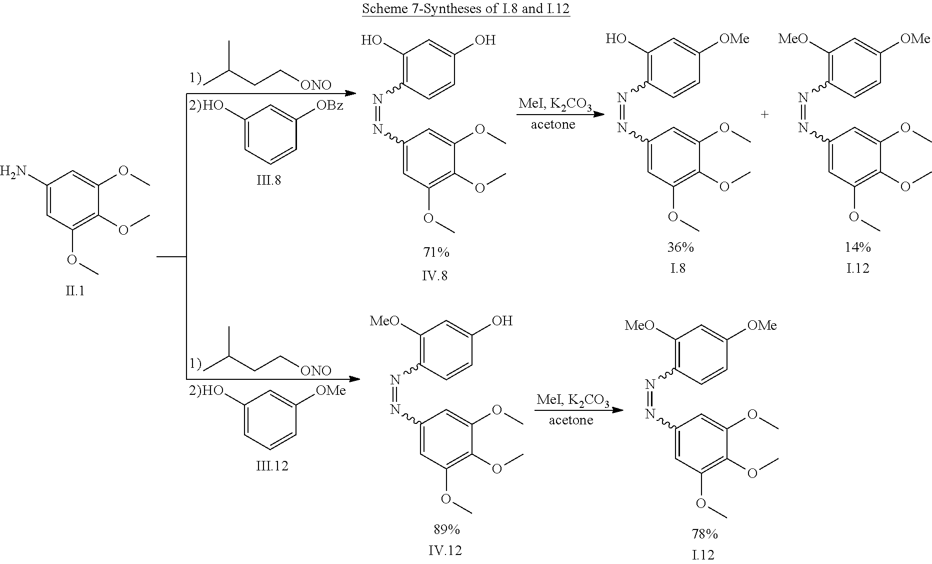

### 4-((3,4,5-trimethoxyphenyl)diazenyl)benzene-1,3-diol (IV.8)

A mono-protected resorcinol could be chosen to reduce byproduct formation during the diazo coupling. By Standard Procedure A, II.1 (590 mg, 3.22 mmol) was reacted with commercial resorcinol monobenzoate (III.8, 724 mg, 3.38 mmol), where the phenol was dissolved in NaOH only one minute prior to diazonium addition to reduce ester hydrolysis prior to reaction, and where the coupling was run for only 15 minutes before neutralisation and extraction. Chromatography of the red crude oil on 5:1->1:1 Hx:EA returned the major product IV.8 (693 mg, 2.28 mmol, 71%; R_{f}=0.37 on 1.7:1 Hx:EA, FeCl₃) as a deep red powder. ¹H-NMR (400 MHz, DMSO): δ=12.15 (s, 1H), 10.50 (s, 1H), 7.68 (d, 8.8 Hz, 1H), 7.26 (s, 2H), 6.49 (dd, 8.8 & 2.5 Hz, 1H), 6.36 (d, 2.5 Hz, 1H), 3.88 (s, 6H), 3.74 (s, 3H) ppm. ¹³C-NMR (100 MHz, DMSO): δ=163.1, 156.6, 153.9 (×2), 147.2, 139.7, 132.5, 129.5, 109.4, 103.5, 99.9 (×2), 60.7, 56.5 (×2) ppm. HRMS (ESI+) calcd for [C₁₅H₁₇N₂O₅]⁺═[MH]⁺: m/z 305.11320. found 305.11322.

### 5-methoxy-2-((3,4,5-trimethoxyphenyl)diazenyl)phenol (I.8)

By Standard Procedure B, IV.8 (670 mg, 2.20 mmol) was methylated overnight with MeI (618 mg, 4.35 mmol, 1.98 eq). Chromatography on a gentle gradient of 10:1->1:1 Hx:EA returned para-monomethylated 1.8 (255 mg, 0.80 mmol, 36%; R_{f}=0.52 on 2.4:1 Hx:EA, FeCl₃) as a red solid, then bismethylated byproduct 1.12 (100 mg, 0.30 mmol, 14%; R_{f}=0.19 on 2.4:1 Hx:EA, FeCl₃) as a viscous red oil. The identity of 1.8 as the para-methoxy isomer was confirmed by comparison to independently synthesised ortho-methoxy isomer IV.12 (detailed below). I.8: ¹H-NMR (400 MHz): δ=7.80 (d, 8.9 Hz, 1H), 7.11 (s, 2H), 6.63 (dd, 8.9 & 2.7 Hz, 1H), 6.50 (d, 2.7 Hz, 1H), 3.97 (s, 6H), 3.94 (s, 3H), 3.89 (s, 3H) ppm. ¹³C-NMR (100 MHz): •=163.8, 156.2, 153.7 (×2), 146.0, 140.0, 134.5, 132.7, 108.3, 101.4, 99.0 (×2), 61.1, 56.3 (×2), 55.7 ppm. LCMS(+): tᵣₑₜ=4.83 min, 319 Th═[MH]⁺: this peak was assigned to the trans isomer since the UV absorption profile did not feature any shoulder band in the visible; ion-sim mode indicated a possible second peak with 319 Th at tᵣₑₜ=3.37 min however no clear UV-Vis spectrum was seen for that peak. HRMS (ESI−) calcd for [C₁₆H₁₇N₂O₅]⁻═[M-H]⁻: m/z 317.11430. found 317.11410.

### 3-methoxy-4-((3,4,5-trimethoxyphenyl)diazenyl)phenol (IV.12)

By Standard Procedure A, II.1 (366 mg, 2.00 mmol) was reacted with commercial 3-methoxyphenol (III.12, 240 mg, 2.05 mmol). Chromatography of the red crude oil on 5:1->1:1 Hx:EA returned IV.12 (564 mg, 1.77 mmol, 89%; R_{f}=0.11 on 2.4:1 Hx:EA, FeCl₃) as a deep red powder. ¹H-NMR (400 MHz, DMSO): δ=10.32 (s br, 1H), 7.55 (d, 8.8 Hz, 1H), 7.12 (s, 2H), 6.60 (d, 2.4 Hz, 1H), 6.45 (dd, 8.8 & 2.4 Hz, 1H), 3.91 (s, 3H), 3.87 (s, 6H), 3.74 (s, 3H) ppm. ¹³C-NMR (100 MHz, DMSO): δ=163.0, 159.3, 153.7 (×2), 149.0, 139.6, 135.2, 118.0, 108.4, 100.4, 100.1 (×2), 60.7, 56.4 (×2), 56.3 ppm. LCMS(+): tᵣₑₜ=3.57 min, 319 Th═[MH]⁺. HRMS (ESI+) calcd for [C₁₆H₁₉N₂O₅]⁺═[MH]⁺: m/z 319.12157. found 319.12856.

### 1-(2,4-di methoxyphenyl)-2-(3,4,5-trimethoxyphenyl)diazene (I.12)

By Standard Procedure B, IV.12 (550 mg, 1.73 mmol) was methylated for 6 hours. Chromatography on a gradient of 5:1->1:1 Hx:EA returned I.12 (460 mg, 1.38 mmol, 78%; R_{f}=0.19 on 2.4:1 Hx:EA, FeCl₃) as an orange powder. ¹H-NMR (400 MHz): δ=7.60 (d, 8.9 Hz, 1H), 7.06 (s, 2H), 6.49-6.39 (m, 2H), 3.88 (s, 3H), 3.83 (s, 6H), 3.78 (s, 3H), 3.76 (s, 3H) ppm. ¹³C-NMR (100 MHz): δ=163.6, 158.5, 153.5 (×2), 149.2, 140.0, 136.7, 118.2, 105.6, 100.1 (×2), 99.0, 61.0, 56.3, 56.2 (×2), 55.6 ppm. LCMS(+): tᵣₑₜ=3.72 and 4.42 min, 333 Th═[MH]⁺: these peaks were assigned to the cis & trans isomers respectively since the UV absorption profile of the first peak (cis) featured a shoulder at around 440 nm which was absent in the second peak. HRMS (ESI+) calcd for [C₁₇H₂₁N₂O₅]⁺═[MH]⁺: m/z 333.14450. found 333.14430.

### North Ring Ortho-Amino, Meta-Hydro Derivative (I.16)

The synthesis of I.16 is presented on Scheme 8 hereafter.

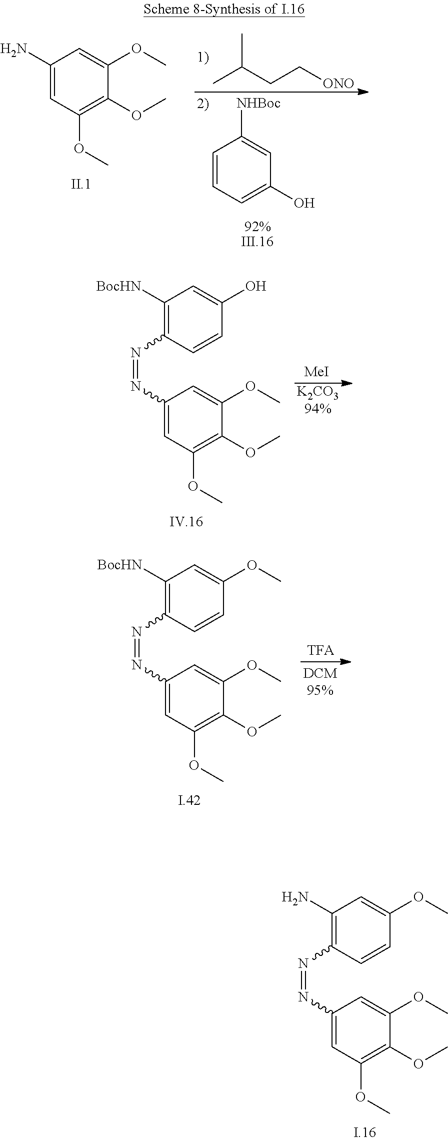

### tert-butyl (5-hydroxy-2-((3,4,5-trimethoxyphenyl)diazenyl)phenyl)carbamate (IV.16)

By Standard Procedure A, II.1 (921 mg, 5.01 mmol) was reacted with III.16 (1.04 g, 4.97 mmol). Chromatography on 5:1->2.4:1 Hx:EA returned IV.16 (1.846 g, 4.57 mmol, 92%; R_{f}=0.44 on 2.4:1 Hx:EA, FeCl₃) as an orange foam. ¹H-NMR (400 MHz, DMSO): δ=10.44 (s br, 1H), 9.84 (s, 1H), 7.70-7.65 (m, 2H), 7.17 (s, 2H), 6.57 (dd, 8.9 & 2.6 Hz, 1H), 3.88 (s, 6H), 3.75 (s, 3H), 1.50 (s, 9H) ppm. ¹³C-NMR (100 MHz, DMSO): δ=162.3, 153.8 (×2), 152.5, 148.4, 139.9, 138.6, 133.1, 123.1, 111.1, 105.6, 100.2 (×2), 80.5, 60.7, 56.3 (×2), 28.3 (×3) ppm. LCMS(+): tᵣₑₜ=4.86 and 5.14 min, each 404 Th═[MH]⁺. HRMS (ESI+) calcd for [C₂₀H₂₆N₃O₆]⁺═[MH⁺]: m/z 404.18161. found 404.18188.

### tert-butyl (5-methoxy-2-((3,4,5-trimethoxyphenyl)diazenyl)phenyl)carbamate (I.42)

By Standard Procedure B, IV.16 (1830 mg, 4.54 mmol) was methylated for 6 hours with MeI (1.13 g, 8.0 mmol) and K₂CO₃ (2.2 g, 16 mmol). Chromatography of the black crude oil on 5:1->2.4:1 Hx:EA returned I.42 (1787 mg, 4.28 mmol, 94%; R_{f}=0.42 and 0.56 on 2.4:1 Hx:EA, trans and cis isomers; FeCl₃) as an orange solid. ¹H-NMR (400 MHz): δ=9.77 (s br, 1H), 7.91 (d, 2.7 Hz, 1H), 7.78 (d, 9.0 Hz, 1H), 7.09 (s, 2H), 6.58 (dd, 9.0 & 2.7 Hz, 1H), 3.89 (s, 6H), 3.86 (s, 3H), 3.85 (s, 3H), 1.48 (s, 9H) ppm. ¹³C-NMR (100 MHz): δ=163.8, 153.6 (×2), 152.4, 147.9, 140.2, 138.6, 132.8, 124.2, 110.0, 101.8, 99.7 (×2), 80.7, 61.1, 56.1, 55.8, 28.3 (×3) Ppm. LCMS(+): tᵣₑₜ=4.72 & 5.81 min, 418 Th═[MH]⁺. HRMS (ESI+) calcd for [C₂₁H₂₇N₃O₆Na]⁺═[MNa⁺]: m/z 440.17921. found 440.17938.

### 5-methoxy-2-((3,4,5-trimethoxyphenyl)diazenyl)aniline (I.16)

To I.42 (1.20 g, 2.87 mmol) were added CH₂Cl₂ (10 mL) and CF₃COOH (12 mL) and the purple solution stirred overnight at room temperature. The volatiles were removed under high vacuum, the purple residue was partitioned between CH₂Cl₂ (30 mL) and K₂HPO₄/KH₂PO₄ buffer (pH=6.8, 30 mL), the aqueous layer extracted with DCM (15 mL), then the combined organic layers were washed with brine (20 mL), dried on Na₂SO₄, filtered and concentrated to give a red crude oil. Chromatography on 5:1->1:1 Hx:EA returned I.16 as a red oil (870 mg, 2.74 mmol, 95%; R_{f}=0.55 on 1:1 Hx:EA (FeCl₃)). ¹H-NMR (400 MHz, CD₃CN): δ=7.67 (d, 8.9 Hz, 1H), 7.18 (s, 2H), 6.48 (s, 2H), 6.36 (dd, 8.9 & 2.7 Hz, 1H), 6.32 (d, 2.6 Hz, 1H), 3.90 (s, 6H), 3.81 (s, 3H), 3.78 (s, 3H) ppm. ¹³C-NMR (100 MHz, CD₃CN): δ=163.1, 153.7 (×2), 149.0, 145.8, 139.1, 131.5, 129.4, 105.2, 99.2 (×2), 99.0, 60.0, 55.7 (×2), 55.1 ppm. LCMS(+): tᵣₑₜ=4.35 min, 318 Th═[MH]+. HRMS (ESI+) calcd for [C₁₆H₂₀N₃O₄]⁺═[MH⁺]: m/z 318.1448. found 318.14454.

### North Ring Ortho-Oxy, Meta-Methyl Derivative (I.17)

The synthesis of I.17 is presented on Scheme 9 hereafter.

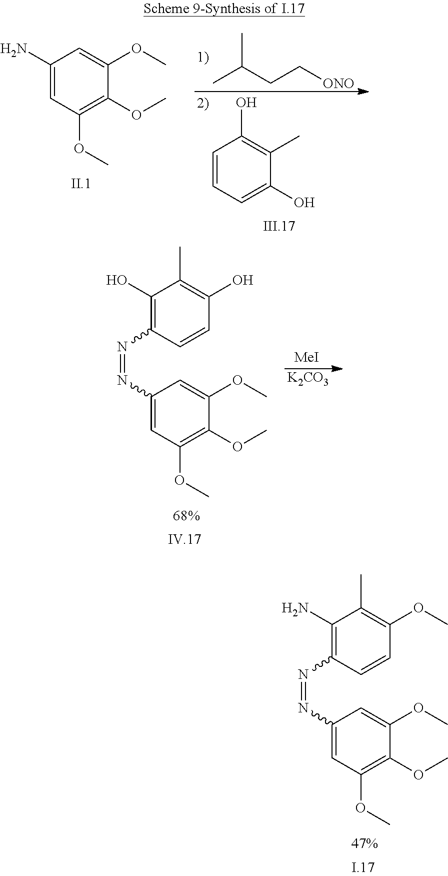

### 2-methyl-4-((3,4,5-trimethoxyphenyl)diazenyl)benzene-1,3-diol (IV.17)

By Standard Procedure A, II.1 (916 mg, 5.01 mmol) was reacted with commercial 2-methylresorcinol (III.17, 618 mg, 4.98 mmol). Chromatography of the red crude oil on 5:1->1:1 Hx:EA returned IV.17 (1079 mg, 3.39 mmol, 68%; R_{f}=0.63 on 1:1 Hx:EA, FeCl₃) as a red solid. ¹H-NMR (400 MHz, DMSO): δ=12.98 (s, 1H), 10.48 (s br, 1H), 7.56 (d, 8.8 Hz, 1H), 7.26 (s, 2H), 6.60 (d, 8.9 Hz, 1H), 3.88 (s, 6H), 3.74 (s, 3H), 2.04 (s, 3H) ppm. ¹³C-NMR (100 MHz, DMSO): δ=160.9, 154.3, 153.9 (×2), 146.7, 139.6, 132.0, 128.7, 111.2, 108.6, 99.7, 60.7, 56.5 (×2), 8.3 ppm. LCMS(+): tᵣₑₜ=4.26 min, 319 Th═[MH]⁺. HRMS (ESI+) calcd for [C₁₆H₁₉N₂O₅]⁺═[MH]⁺: m/z 319.12157. found 319.12859.

### 3-methoxy-2-methyl-6-((3,4,5-trimethoxyphenyl)diazenyl)phenol (I.17)

By Standard Procedure B, IV.17 (1.05 g, 3.30 mmol) was methylated for four hours with MeI (1.02 eq). Chromatography on a gradient of 5:1->1:1 Hx:EA returned I.17 (520 mg, 1.56 mmol, 47%; R_{f}=0.51 on 2.4:1 Hx:EA, FeCl₃) as a red-orange solid which could be crystallised as fine red needles from Hx/EtOAc. ¹H-NMR (400 MHz): δ=7.67 (d, 8.9 Hz, 1H), 7.04 (s, 2H), 6.57 (d, 8.9 Hz, 1H), 3.88 (s, 6H), 3.86 (s, 3H), 3.85 (s, 3H), 2.08 (s, 3H) ppm. ¹³C-NMR (100 MHz): δ=161.4, 153.7 (×2), 152.9, 146.3, 139.9, 132.9, 132.0, 113.6, 102.8, 99.1 (×2), 61.1, 56.2 (×2), 55.8, 7.57 ppm. LCMS(+): tᵣₑₜ=5.24 min, 333 Th═[MH]⁺; this peak was attributed as the trans-isomer due to the single band structure with absorption maximum at 390 nm; no peak for the cis-isomer could be observed even after pre-irradiation of the sample with 390 nm before injection. HRMS (ESI+) calcd for [C₁₇H₂₁N₂O₅]⁺═[MH]⁺: m/z 333.14450. found 333.14421.

### North Ring Ortho- and/or Meta-Fluoro Derivatives (I.3), (I.4) and (I.5)

The syntheses of I.3, I.4 and I.5 are presented on Scheme 10 hereafter.

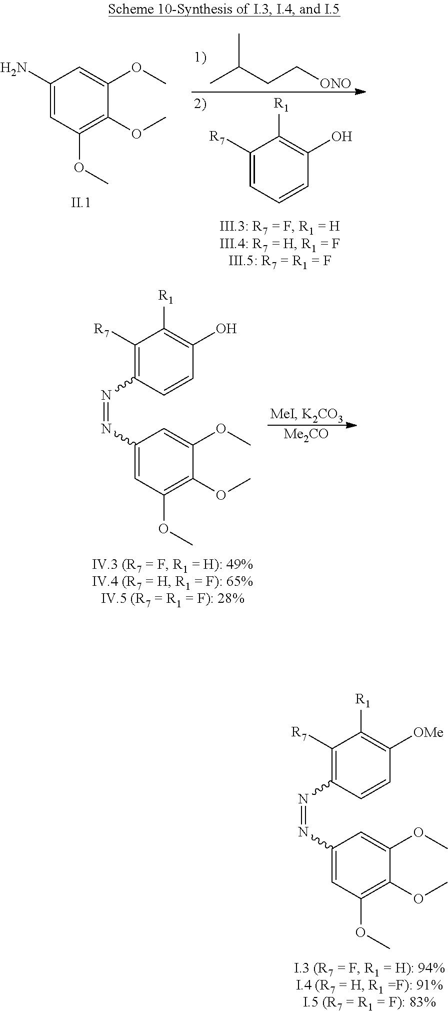

### 3-fluoro-4-((3,4,5-trimethoxyphenyl)diazenyl)phenol (IV.3)

By Standard Procedure A, II.1 (196 mg, 1.07 mmol) was reacted with commercial 3-fluorophenol (III.3; 120 mg, 1.07 mmol), and the product was extracted with ethyl acetate. Chromatography on 5:1->2.5:1 Hx:EA returned IV.3 (161 mg, 0.53 mmol, 49%; R_{f}=0.20 on 2.4:1 Hx:EA, KMnO₄) as a yellow oil only sparingly soluble in CH₂Cl₂ or CHCl₃. ¹H-NMR (400 MHz): δ=7.68-7.60 (m, 2H), 7.14 (s, 2H), 7.07 (˜t, 8.8 Hz, 1H), 5.47 (d br, 4.2 Hz, 1H), 3.89 (s, 6H), 3.86 (s, 3H) ppm. ¹³C-NMR (100 MHz) showed the expected C—F couplings, as did the spectra of the other fluorinated compounds: 5=153.5 (×2), 151.3 (d, 240.1 Hz), 148.2, 146.7 (d, 5.2 Hz), 146.1 (d, 15.3 Hz), 140.5, 122.6 (d, 2.9 Hz), 117.0 (d, 2.2 Hz), 107.8 (d, 19.3 Hz), 100.3 (×2), 61.1, 56.2 (×2) ppm. HRMS (ESI−) calcd for [C₁₅H₁₄N₂O₄F]⁻═[M-H]⁻: m/z 305.09431. found 305.09427.

### 1-(2-fluoro-4-methoxyphenyl)-2-(3,4,5-trimethoxyphenyl)diazene (I.3)

By Standard Procedure B, IV.3 (159 mg, 0.52 mmol) was methylated overnight in a mixture of acetone (15 mL), EA (0.6 mL), CHCl₃ (0.7 mL) and DMSO (0.7 mL). Chromatography on 10:1->4:1 Hx:EA cleanly returned I.3 (158 mg, 0.49 mmol, 94%; R_{f}=0.44 and 0.20 on 2.4:1 Hx:EA, FeCl₃: trans and cis isomers) as a red oil. ¹H-NMR (400 MHz, DMSO): δ=7.83 (ddd, 8.7 & 2.4 & 1.2 Hz, 1H), 7.69 (dd, 12.4 & 2.3 Hz, 1H), 7.39 (˜t, 8.9 Hz, 1H), 7.24 (s, 2H), 3.96 (s, 3H), 3.89 (s, 6H), 3.77 (s, 3H) ppm. ¹³C-NMR (100 MHz, DMSO): δ=153.85 (×2), 152.3 (d, 247.1 Hz), 150.3 (d, 11.1 Hz), 148.0, 146.0 (d, 5.1 Hz), 140.6, 123.3 (d, 2.9 Hz), 114.1 (d, 2.2 Hz), 107.4 (d, 19.1 Hz), 100.6 (×2), 60.7, 56.8, 56.5 (×2) ppm. ¹⁹F-NMR (282 MHz, DMSO): δ=−133.45 (ddd, 12.2 & 10.2 & 1.3 Hz) ppm. LCMS(+): tᵣₑₜ=3.86 & 4.80 min, each 321 Th═[MH]⁺: these peaks were assigned to the cis & trans isomers respectively since the UV absorption profile of the first peak (cis) featured a shoulder centred at 440 nm which was absent in the second peak. HRMS (EI+) calcd for [C₁₆H₁₇N₂O₄F]⁺═[M]⁺: m/z 320.1172. found 320.1170.

### 2-fluoro-4-((3,4,5-trimethoxyphenyl)diazenyl)phenol (IV.4)

By Standard Procedure A, II.1 (183 mg, 1.00 mmol) was reacted with commercial 2-fluorophenol (III.4; 116 mg, 1.04 mmol). Chromatography on 5:1->2.5:1 Hx:EA returned IV.4 (198 mg, 0.65 mmol, 65%; R_{f}=0.25 on 2.4:1 Hx:EA, KMnO₄) as a yellow oil. ¹H-NMR (400 MHz, DMSO): δ=10.77 (s, 1H), 7.68 (˜t, 8.9 Hz, 1H), 7.18 (s, 2H), 6.80 (dd, 12.7 & 2.5 Hz, 1H), 6.74 (dd, 8.9 & 2.5 Hz, 1H), 3.88 (s, 6H), 3.76 (s, 3H) ppm. ¹³C-NMR (100 MHz, DMSO): δ=162.8 (d, 12.1 Hz), 161.2 (d, 255.7 Hz), 153.8 (×2), 148.6, 140.3, 133.4 (d, 6.9 Hz), 119.0 (d, 2.0 Hz), 112.9 (d, 2.4 Hz), 103.8 (d, 21.8 Hz), 100.4 (×2), 60.7, 56.4 (×2) ppm. HRMS (ESI−) calcd for [C₁₅H₁₄N₂O₄F]⁻═[M-H]⁻: m/z 305.09431. found 305.09433.

### 1-(3-fluoro-4-methoxyphenyl)-2-(3,4,5-trimethoxyphenyl)diazene (I.4)

By Standard Procedure B, IV.4 (190 mg, 0.62 mmol) was methylated overnight. Chromatography on 10:1->4:1 Hx:EA cleanly returned I.4 (173 mg, 0.54 mmol, 91%; R_{f}=0.42 and 0.25 on 2.4:1 Hx:EA, FeCl₃: trans and as isomers) as fine orange crystals. ¹H-NMR (400 MHz, DMSO): δ=7.75 (˜t, 9.0 Hz, 1H), 7.22 (s, 2H), 7.12 (dd, 13.0 & 2.6 Hz, 1H), 6.92 (ddd, 9.2 & 2.7 & 0.9 Hz, 1H), 3.89 (s, 6H), 3.89 (s, 3H), 3.77 (s, 3H) ppm. ¹³C-NMR (100 MHz, DMSO): δ=163.8 (d, 11.2 Hz), 161.1 (d, 255.9 Hz), 153.8 (×2), 148.5, 140.6, 134.3 (d, 7.2 Hz), 118.8 (d, 2.1 Hz), 112.0 (d, 2.6 Hz), 102.7 (d, 23.4 Hz), 100.6 (×2), 60.7, 56.7, 56.4 (×2) ppm. ¹⁹F-NMR (282 MHz, DMSO): δ=−121.31 (ddd, 13.2 & 8.8 & 1.1 Hz) ppm. LCMS(+): tᵣₑₜ=3.96 & 4.82 min, each 321 Th═[MH]⁺: these peaks were assigned to the cis & trans isomers respectively since the UV absorption profile of the first peak (cis) featured a shoulder centred at 445 nm which was absent in the second peak. HRMS (EI+) calcd for [C₁₆H₁₇N₂O₄F]⁺═[M]⁺: m/z 320.1172. found 320.1167.

### 2,3-difluoro-4-((3,4,5-trimethoxyphenyl)diazenyl)phenol (IV.5)

By Standard Procedure A, II.1 (186 mg, 1.02 mmol) was reacted with commercial 2,3-difluorophenol (III.5; 140 mg, 1.08 mmol). Chromatography on 5:1->2.5:1 Hx:EA returned IV.5 (91 mg, 0.28 mmol, 28%; R_{f}=0.26 on 1.7:1 Hx:EA, Van) as a yellow oil. ¹H-NMR (400 MHz): δ=7.46 (ddd, 9.2 & 7.5 & 2.3 Hz, 1H), 7.17 (s, 2H), 6.78 (ddd, 9.3 & 8.0 & 2.1 Hz, 1H), 5.79 (s br, 1H), 3.89 (s, 6H), 3.87 (s, 3H) ppm. ¹³C-NMR (100 MHz): δ=153.5 (×2), 149.1 (dd, 260.6 & 11.3 Hz), 148.6, 147.3 (d, 11.4 Hz), 140.4 (dd, 240.2 & 13.4 Hz), 140.9, 135.4 (d, 4.7 Hz), 112.7 (d, 3.7 Hz), 111.9 (d, 3.6 Hz), 100.6 (×2), 61.1, 56.24 (×2) ppm. HRMS (ESI−) calcd for [C₁₅H₁₃N₂O₄F₂]⁻═[M-H]⁻: m/z 323.08489. found 323.08495.

### 1-(2,3-difluoro-4-methoxyphenyl)-2-(3,4,5-trimethoxyphenyl)diazene (I.5)

By Standard Procedure B, IV.5 (89 mg, 0.27 mmol) was methylated overnight. Chromatography on 10:1->2:1 Hx:EA cleanly returned I.5 (79 mg, 0.23 mmol, 83%; R_{f}=0.34 and 0.17 on 2.4:1 Hx:EA, FeCl₃: trans and as isomers) as fine orange crystals. ¹H-NMR (400 MHz, DMSO): δ=7.59 (ddd, 9.3 & 8.0 & 2.3 Hz, 1H), 7.23 (s, 2H), 7.19 (ddd, 9.7 & 8.0 & 1.9 Hz, 1H), 3.98 (s, 3H), 3.89 (s, 6H), 3.78 (s, 3H) ppm. ¹³C-NMR (100 MHz, DMSO): δ=153.8 (×2), 151.3 (dd, 7.8 & 3.1 Hz), 148.7 (dd, 255.9 & 10.8 Hz), 148.3, 141.1, 140.9 (dd, 246.4 & 12.9 Hz), 134.9 (d, 4.8 Hz), 112.8 (d, 3.9 Hz), 109.2 (d, 3.1 Hz), 100.8 (×2), 60.7, 57.4, 56.5 (×2) ppm. ¹⁹F-NMR (282 MHz, DMSO): δ=−148.54 (ddd, 20.0 & 8.1 & 2.1 Hz), −159.65 (ddd, 20.1 & 8.1 & 2.5 Hz) ppm. LCMS(+): tᵣₑₜ=4.09 & 4.84 min, each 338 Th=[MH]⁺: these peaks were assigned to the cis & trans isomers respectively since the UV absorption profile of the first peak (cis) had a shoulder centred at 440 nm which was absent in the second peak. HRMS (EI+) calcd for [C₁₆H₁₆N₂O₄F₂]⁺═[M]⁺: m/z 338.1078. found 338.1074.

### South Ring Bis(Meta-Desoxyfluoro) Derivative (I.9)

The synthesis of I.9 is presented on Scheme 11 hereafter.

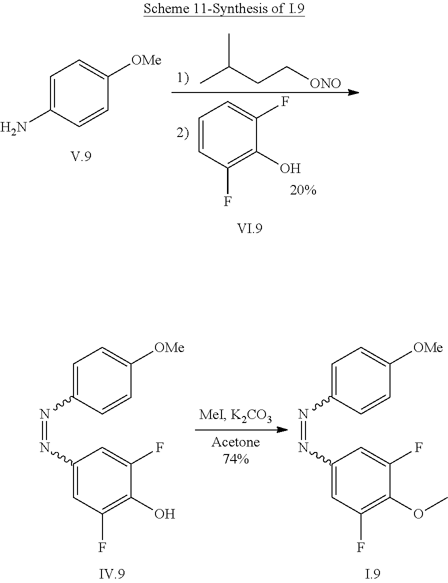

### 2,6-difluoro-4-((4-methoxyphenyl)diazenyl)phenol (IV.9)

By Standard Procedure A, commercial para-anisidine (V.9; 185 mg, 1.50 mmol) was reacted with commercial 2,6-difluorophenol (VI.9; 199 mg, 1.53 mmol), with stirring at 0° C. continued for 2 hours to allow for greater reaction completion. Chromatography of the dark red crude oil on 5:1->2.5:1 Hx:EA returned IV.9 (80 mg, 0.30 mmol, 20%; R_{f}=0.49 on 2.4:1 Hx:EA, Van) as a red solid. ¹H-NMR (400 MHz): δ=7.81 (d, 9.0 Hz, 2H), 7.47 (d, 8.9 Hz, 2H), 6.94 (d, 9.1 Hz, 2H), 3.83 (s, 3H) ppm. ¹³C-NMR (100 MHz): 5=162.4, 151.7 (dd, 243.7 & 6.1 Hz; ×2), 146.4, 145.1 (t, 7.2 Hz), 134.7 (t, 16.8 Hz), 124.9 (×2), 114.3 (×2), 106.6-106.30 (m, ×2), 55.63 ppm. ¹⁹F-NMR (282 MHz): δ=−134.75 (d, 8.8 Hz) ppm. HRMS (ESI+) calcd for [C₁₃H₁₁N₂O₂F₂]⁺═[MH]⁺: m/z 265.07831. found 265.07832. LCMS(+): tᵣₑₜ=4.46 min, 265 Th═[MH]⁺.

### 1-(3,5-difluoro-4-methoxyphenyl)-2-(4-methoxyphenyl)diazene (I.9)

By Standard Procedure B, IV.9 (75 mg, 0.28 mmol) was methylated overnight. Chromatography of the orange crude oil on 7.5:1->5:1 Hx:EA returned I.9 (58 mg, 0.21 mmol, 74%; R_{f}=0.78 & 0.68 on 2.4:1 Hx:EA, trans & cis isomers, FeCl₃) as an orange oil. ¹H-NMR (400 MHz): δ=7.89 (d, 9.0 Hz, 2H), 7.63 (d, 9.5 Hz, 2H), 7.15 (d, 9.1 Hz, 2H), 4.03 (t, 1.3 Hz, 3H), 3.88 (s, 3H) ppm. ¹³C-NMR (100 MHz): δ=163.0, 155.5 (dd, 248.6 & 6.7 Hz; ×2), 147.0 (t, 8.0 Hz), 146.1, 138.1 (t, 14.5 Hz), 125.4 (×2), 115.2 (×2), 107.3-106.9 (m; ×2), 62.3, 56.2 ppm. ¹⁹F-NMR (282 MHz, DMSO): δ=−127.35 (dd, 9.6 & 1.3 Hz) ppm. HRMS (EI+) calcd for [C₁₄H₁₃N₂O₂F₂]⁺═[M]⁺: m/z 278.0867. found 278.0873. LCMS(+): tᵣₑₜ=4.36 & 5.44 min, 279 Th═[MH]⁺, cis and trans isomers respectively (cis isomer has a shoulder at 440 nm).

### North Ring Ortho-Fluoro, Meta-Nitro Derivative (I.7)

The synthesis of I.7 is presented on Scheme 12 hereafter.

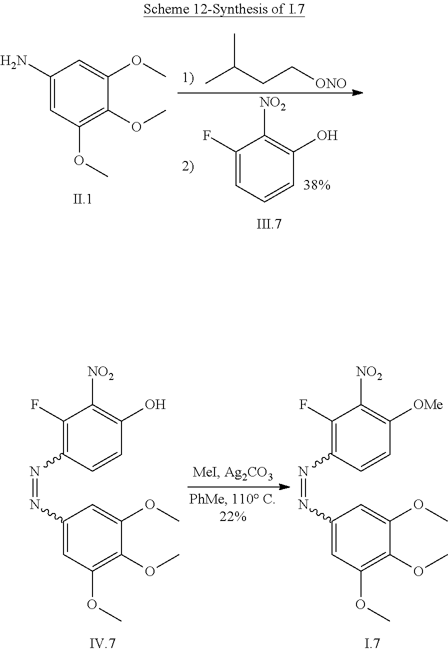

### 3-fluoro-2-nitro-4-((3,4,5-trimethoxyphenyl)diazenyl)phenol (IV.7)

By Standard Procedure A, II.1 (185 mg, 1.01 mmol) was reacted with 3-fluoro-2-nitrophenol (III.7; 162 mg, 1.03 mmol), where the reaction between the phenolate and the diazonium was stirred for 5 h in the dark in the cold to allow for better conversion of the slow-reacting materials. Chromatography on 1:1:0->5:1:0->5:1:0.5 Hx:EA:MeOH returned IV.7 (134 mg, 0.38 mmol, 38%; R_{f}=0.16 on 1:5 Hx:EA, Han) as a red solid. ¹H-NMR (400 MHz): δ=10.68 (s, 1H), 8.02 (dd, 9.4 & 7.6 Hz, 1H), 7.26 (s, 2H), 6.98 (dd, 9.4 & 1.9 Hz, 1H), 3.90 (s, 6H), 3.88 (s, 3H) ppm. ¹³C-NMR (100 MHz): δ=157.4 (˜s), 155.8 (d, 279 Hz), 153.6 (×2), 148.3, 141.5, 134.4 (d, 6.3 Hz), 125.8 (d, 7.4 Hz), 125.0 (d, 3.3 Hz), 114.7 (d, 4.3 Hz), 100.9 (×2), 61.10, 56.3 (×2) ppm. LCMS(+): tᵣₑₜ=4.25, 352 Th═[MH]⁺. HRMS (ESI−) calcd for [C₁₅H₁₃N₃O₆F]⁻═[M-H]⁻: m/z 350.0794. found 350.0796.

### 1-(2-fluoro-4-methoxy-3-nitrophenyl)-2-(3,4,5-trimethoxyphenyl)diazene (I.7)

By Standard Procedure C, IV.7 (130 mg, 0.37 mmol) was methylated using MeI (69 mg, 0.48 mmol) and Ag₂CO₃ (50% on Celite, 210 mg, 0.38 mmol) in toluene (6 mL). The crude was separated with 3:1->2:1 Hx:EA to afford 1.7 (30 mg, 0.08 mmol, 22%; R_{f}=0.19 on 2.4:1 Hx:EA, FeCl₃) as an orange solid. ¹H-NMR (400 MHz): δ=7.85 (dd, 9.3 & 8.1 Hz, 1H), 7.17 (s, 2H), 6.84 (dd, 9.4 & 1.8 Hz, 1H), 3.94 (s, 3H), 3.89 (s, 6H), 3.87 (s, 3H) ppm. ¹³C-NMR (100 MHz): δ=153.8 (d, 2.6 Hz), 153.6 (×2), 152.2 (d, 270 Hz), 148.4, 141.4, 134.5 (d, 6.1 Hz), 131.4 (d, 14.6 Hz), 120.0 (d, 2.1 Hz), 107.7 (d, 3.6 Hz), 100.8 (×2), 61.1, 57.2, 56.2 (×2) ppm. ¹⁹F-NMR (282 MHz): δ=−132.69 (dd, 8.1 & 1.9 Hz) ppm. LCMS(+): tᵣₑₜ=4.18 & 4.83 min, each 366 Th═[MH]⁺: these peaks were assigned to the cis & trans isomers respectively since the first peak showed a shoulder at 445 nm whereas the second peak had a single-band structure. HRMS (ESI+) calcd for [C₁₆H₁₇N₃O₆F]⁺═[MH⁺]: m/z 366.1096. found 366.10944.

### North Ring Heteroarylic, Meta-Pyridinyl, Derivative (I.13)

The synthesis of I.13 is presented on Scheme 13 hereafter.

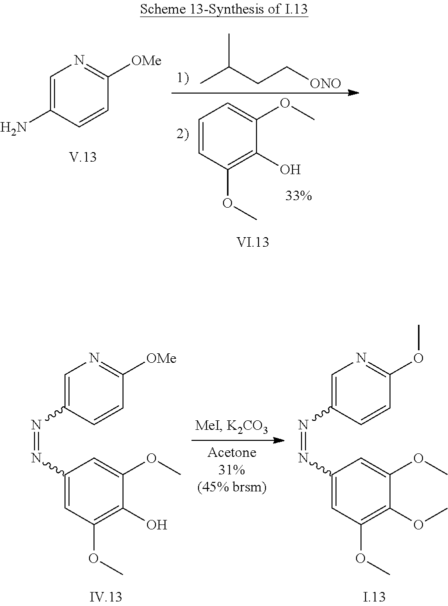

### 2,6-dimethoxy-4-((6-methoxypyridin-3-yl)diazenyl)phenol (IV.13)

By Standard Procedure A, 6-methoxypyridin-3-amine (V.13; 260 mg, 2.10 mmol) was reacted with 2,6-dimethoxyphenol (VI.13; 316 mg, 2.05 mmol). Chromatography of the red crude oil on 5:1->2.5:1 Hx:EA returned IV.13 (195 mg, 0.67 mmol, 33%; R_{f}=0.23 on 2.4:1 Hx:EA, FeCl₃) as an orange solid. ¹H-NMR (400 MHz, DMSO): δ=9.31 (s br, 1H), 8.75 (dd, 2.6 & 0.6 Hz, 1H), 8.11 (dd, 9.0 & 2.6 Hz, 1H), 7.24 (s, 2H), 6.98 (dd, 8.9 & 0.6 Hz, 1H), 3.96 (s, 3H), 3.87 (s, 6H) ppm. ¹³C-NMR (100 MHz, DMSO): δ=165.2, 148.6 (×2), 146.6, 144.5, 143.9, 139.9, 129.1, 112.1, 101.1 (×2), 56.5 (×2), 54.3 ppm. HRMS (ESI+) calcd for [C₁₄H₁₆N₃O₄]⁺═[MH]⁺: m/z 290.11353. found 290.11351.

### 2-methoxy-5-((3,4,5-trimethoxyphenyl)diazenyl)pyridine (I.13)

By Standard Procedure B, IV.13 (191 mg, 0.66 mmol) was methylated overnight. Chromatography on 3:1->2.4:1 Hx:EA returned I.13 (61 mg, 0.20 mmol, 31% or 45% brsm; R_{f}=0.54 on 2.4:1 Hx:EA, FeCl₃) as an orange solid, followed by unreacted IV.13 (64 mg, 0.22 mmol). ¹H-NMR (400 MHz): δ=8.73 (dd, 2.6 & 0.6 Hz, 1H), 8.04 (dd, 8.9 & 2.6 Hz, 1H), 7.16 (s, 2H), 6.77 (dd, 8.9 & 0.6 Hz, 1H), 3.97 (s, 3H), 3.90 (s, 6H), 3.87 (s, 3H) ppm. ¹³C-NMR (100 MHz): δ=165.6, 153.5 (×2), 148.5, 147.6, 143.8, 140.6, 128.5, 111.8, 100.3 (×2), 61.1, 56.2 (×2), 54.1 ppm. LCMS(+): tᵣₑₜ=3.57 & 4.65 min, each 304 Th═[MH]⁺: these peaks were assigned to the cis & trans isomers respectively since the UV absorption profile of the first peak (ds) featured a shoulder centred around 440 nm which was absent in the second peak. HRMS (ESI+) calcd for [C₁₅H₁₈N₃O₄]⁺═[MH]⁺: m/z 304.12918. found 304.12919.

### North Ring Heteroarylic, Quinolinyl, Derivative (I.14)

The synthesis of I.14 is presented on Scheme 14 hereafter.

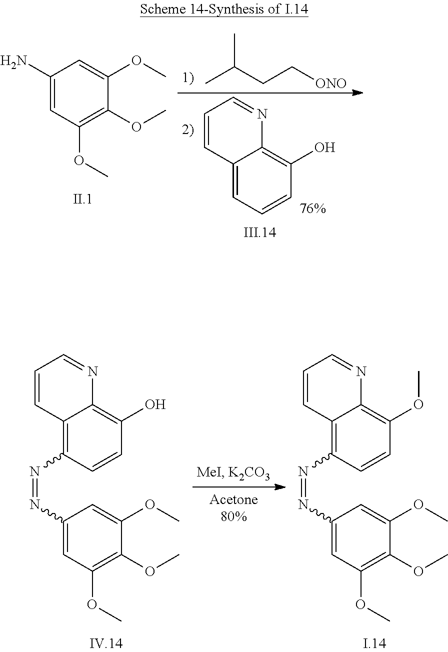

### 5-((3,4,5-trimethoxyphenyl)diazenyl)quinolin-8-ol (IV.14)

By Standard Procedure A, II.1 (366 mg, 2.00 mmol) was reacted with commercial 8-hydroxyquinoline (III.14; 300 mg, 2.07 mmol). Chromatography of the red crude solid on 1:1:0->1:5:0->1:5:0.3 Hx:EA:MeOH returned IV.14 (514 mg, 1.52 mmol, 76%; R_{f}=0.07 on 1:5 Hx:EA, FeCl₃) as a deep orange solid. ¹H-NMR (400 MHz): δ=9.20 (dd, 8.5 & 1.4 Hz, 1H), 8.81 (dd, 4.2 & 1.6 Hz, 1H), 7.95 (d, 8.3 Hz, 1H), 7.56 (dd, 8.6 & 4.1 Hz, 1H), 7.26 (s, 2H), 7.20 (d overlapped, ˜7 Hz, 1H), 3.93 (s, 6H), 3.88 (s, 3H) ppm. ¹³C-NMR (100 MHz) showed 2 isomers in >4:1 ratio, only the major isomer's peaks are reported: 5=155.2, 153.6 (×2), 149.0, 148.5, 140.5, 139.9, 137.7, 132.9, 127.0, 122.8, 115.6, 110.1, 100.3 (×2), 61.1, 56.3 (×2) ppm. HRMS (ESI+) calcd for [C₁₈H₁₈N₃O₄]⁺═[MH]⁺: m/z 340.12918. found 340.12898. LCMS(+): tᵣₑₜ=4.02 min, 340 Th═[MH]⁺.

### 8-methoxy-5-((3,4,5-trimethoxyphenyl)diazenyl)quinoline (I.14)

By Standard Procedure B, IV.14 (505 mg, 1.49 mmol) was methylated overnight. Chromatography of the red crude on 1:5:0->1:5:1 Hx:EA:MeOH returned I.14 (422 mg, 1.20 mmol, 80%; R_{f}=0.32 on 1:5:0.1 Hx:EA:MeOH, FeCl₃) as a brown solid. ¹H-NMR (400 MHz): δ=9.25 (d, 8.5 Hz, 1H), 9.03-8.97 (m, 1H), 7.92 (d, 8.6 Hz, 1H), 7.59 (dd, 8.6 & 3.8 Hz, 1H), 7.25 (s, 2H), 7.12 (d, 8.6 Hz, 1H), 4.13 (s, 3H), 3.94 (s, 6H), 3.89 (s, 3H) ppm. ¹³C-NMR (100 MHz): δ=157.5, 153.6 (×2), 149.4, 149.4, 149.0, 140.7, 140.7, 133.1, 127.8, 122.5, 114.0, 107.7, 100.5 (×2), 61.1, 56.5, 56.3 (×2) ppm. LCMS(+): tᵣₑₜ=2.69 & 3.55 min, each 354 Th═[MH]⁺: these peaks were assigned to the cis & trans isomers respectively since the UV absorption profile of the first peak (cis) featured a very broad shoulder centred around 400 nm and extending to 480 nm at half-maximum, which was absent in the second peak. HRMS (ESI+) calcd for [C₁₉H₂₀N₃O₄]⁺═[MH]⁺: m/z 354.14483. found 354.14462.

### North Ring Heteroarylic, Quinolinyl, Derivative (I.15)

The synthesis of I.15 is presented on Scheme 15 hereafter.

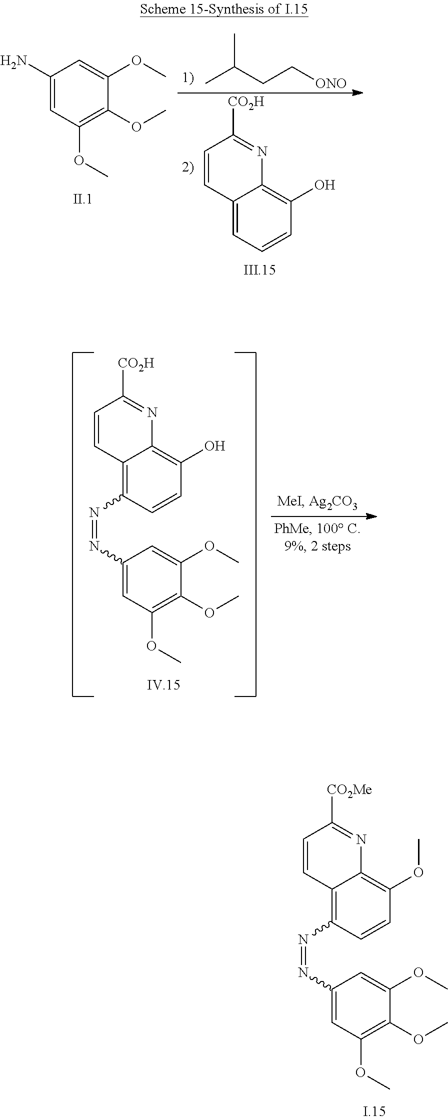

### methyl 8-methoxy-5-((3,4,5-trimethoxyphenyl)diazenyl)quinoline-2-carboxylate (I.15)

By Standard Procedure A, II.1 (187 mg, 1.02 mmol) was reacted with commercial 8-hydroxyquinoline-2-carboxylic acid (III.15; 195 mg, 1.03 mmol), using EtOAc as the extraction solvent while maintaining the aqueous phase at pH=3. Pad filtration of the black crude on 1:0->1:1 CHCl₃:MeOH removed both fast-running and immobile crude components, and the resultant black crude IV.15 containing residual III.15 and other contaminants was carried over to the next step directly (247 mg; LCMS(+): tᵣₑₜ=4.02 min, 384 Th═[MH]⁺). Crude IV.15 (120 mg) was methylated by Standard Procedure C using MeI (88 mg, >2 equivalents). Chromatography on 5:1:0->1:1:0->1:1:1 Hx:EA:MeOH returned I.15 (19 mg, 0.046 mmol; 9% over 2 steps; R_{f}=0.27 on 1:5 Hx:EA, UV) as an orange oil. ¹H-NMR (400 MHz) without precautions to block ambient light revealed a 3:1 proportion of [presumably trans:cis] isomers: δ=9.37 (d, 8.9 Hz, 1H_{Z}), 9.34 (d, 8.8 Hz, 1H_{E}), 9.04 (d, 8.7 Hz, 1H_{Z}), 8.33 (d, 8.8 Hz, 1H_{E}), 8.29 (d, 8.8 Hz, 1H_{Z}), 7.99 (d, 8.6 Hz, 1H_{E}), 7.58 (s, 2H_{Z}), 7.25 (s, 2H_{E}), 7.13 (d, 8.6 Hz, 1H_{E}), 7.10 (d, 8.9 Hz, 1H_{Z}), 4.12 (s, 3H_{E}), 4.11 (s, 3H_{Z}), 4.02 (s, 3H_{E}), 4.01 (s, 3H_{Z}), 3.94 (s, 6H_{E}), 3.94 (s, 6H_{Z}), 3.89 (s, 3H_{E}), 3.88 (s, 3H_{Z}) ppm. ¹³C-NMR (100 MHz; only major isomer peaks are reported): δ=165.9, 158.4, 153.6 (×2), 148.9, 147.2, 140.8, 140.5, 139.1, 133.6, 128.9, 122.3, 116.0, 107.8, 100.5 (×2), 61.1, 56.6, 56.3 (×2), 53.2 ppm. HRMS (ESI+) calcd for [C₂₁H₂₂N₃O₆]⁺═[MH]⁺: m/z 412.15031. found 412.15036. LCMS(+): tᵣₑₜ=3.61 & 4.47 min, 412 Th═[MH]⁺: these peaks were assigned to the cis & trans isomers respectively since the UV absorption profile of the first peak (cis) featured a shoulder centred around 455 nm.

### (I.10)—a Water-Soluble, Peptidase-Activatable Prodrug of (I.1)

The synthesis of I.10 is presented on Scheme 16 hereafter.

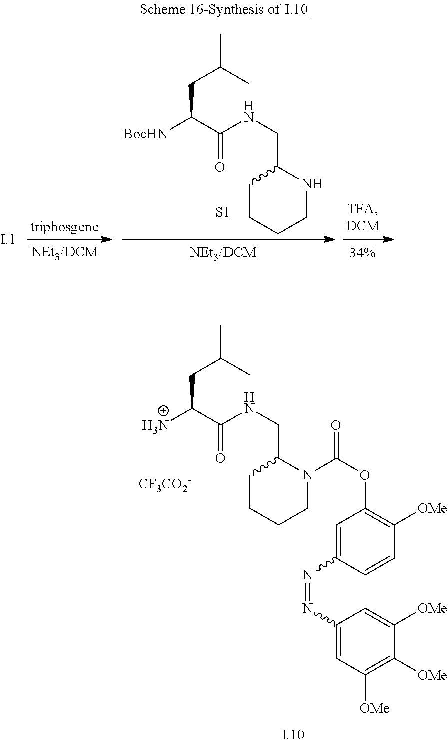

Caution: phosgene, liberated by amine-mediated decomposition of triphosgene, has boiling point 8° C., is highly toxic and corrosive and can react violently with water or other nucleophiles especially if the reaction is in homogenous media. Reactions were kept cold to avoid boil-off of phosgene. Excess phosgene was caught apparently quantitatively during evaporation in a primary liquid nitrogen trap (a backup trap was employed but always found empty); it was destroyed when still cold by its dropwise addition to a vigorously stirred, cold mixture of 2-aminoethanol or piperidine (1 mL) and ethanol (5 mL) in dichloromethane (20 mL) in a well-ventilated hood.

### (2-methoxy-5-((3,4,5-trimethoxyphenyl)diazenyl)phenyl) 2-((L-leucinamido)methyl)piperidine-1-carboxylate 2,2,2-trifluoroacetate salt (I.10)

To a solution of I.1 (13 mg, 0.041 mmol) in CH₂Cl₂ (3 mL) under nitrogen atmosphere in an ice bath, were added a solution of triphosgene (60 mg, 0.20 mmol) in CH₂Cl₂ (1 mL), then, dropwise, triethylamine (0.10 mL). The solution was stirred in the cold for 30 min then the volatiles were evaporated at high vacuum. To the residue under nitrogen were added a solution of NEt₃ (0.15 mL) and N-tert-butoxycarbonyl-L-leucyl-(piperidin-2-ylmethyl)amide (S1, 16 mg, 0.045 mmol; prepared according to known procedure^{[22]}) in CH₂Cl₂ (3 mL), and the mixture stirred for 2 h at room temperature. The volatiles were evaporated, and a solution of TFA (2 mL) in DCM (2 mL) was added. The purple solution was stirred at room temperature for 30 min. The volatiles were removed at 0.4 mbar until the purple residue had become yellow-brown, indicating removal of excess TFA. Chromatography on 5:1:0->1:1:0->1:1:1 Hx: EA: MeOH returned I.10 2,2,2-trifluoroacetate salt (9.5 mg, 0.014 mmol, 34%; R_{f}=0.54 on 1:1:1 Hx:EA:MeOH, FeCl₃) as a brown viscous oil. ¹H-NMR (400 MHz, DMSO): δ=7.87 (dd, 8.7 & 2.4 Hz, 1H), 7.63 (d, 2.5 Hz, 1H), 7.32 (d, 8.9 Hz, 1H), 7.23 (s, 2H), 6.21 (s, 1H), 3.90 (s, 3H), 3.90 (s, 3H), 3.89 (s, 6H), 4.51-4.23 (m, 2H), 3.86-3.02 (m, 4H overlapped), 1.82-1.61 (m, 1H), 1.58-1.34+1.12-1.06 (m+m, 8H), 0.85-0.80 (m, 6H) ppm. LCMS(+): tᵣₑₜ=2.92 & 3.41 min, each 572 Th═[MH]⁺; the first peak was assigned as the as isomer due to its absorbance shoulder centred at 450 nm. HRMS (ESI+) calcd for [C₂₉H₄₂N₅O₇]⁺═[MH]⁺: m/z 572.30788. found 572.30867.

### “Azoombrabulin” (I.20)—a Water-Soluble Prodrug of (I.2)

The synthesis of I.20 is presented on Scheme 17 hereafter.

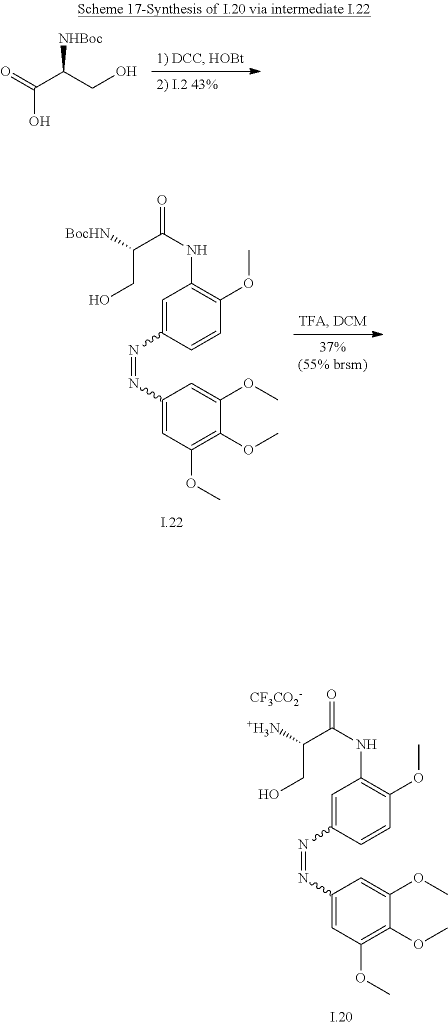 [SMILES: CC(C)(C)OC(=O)N[C@@H](CO)C(=O)O ; COc1ccc(N=Nc2cc(OC)c(OC)c(OC)c2)cc1NC(=O)[C@H](CO)NC(=O)OC(C)(C)C ; "CF3CO2-" ; COc1ccc(N=Nc2cc(OC)c(OC)c(OC)c2)cc1NC(=O)[C@@H]([NH3+])CO]

### (2-methoxy-5-((3,4,5-trimethoxyphenyl)diazenyl)phenyl))-1-(N-tert-butoxycarbonyl)L-serinamide (I.22)

To commercial N-tert-butoxycarbonyl-L-serine (65 mg, 0.32 mmol) in an icebath were added DCM (15 mL), HOBt (50 mg, 0.37 mmol) and DCC (68 mg, 0.33 mmol), and the mixture stirred for 5 min. I.2 (100 mg, 0.31 mmol) was added, and stirring continued overnight while warming slowly to room temperature. The organic phase was washed with saturated aqueous sodium carbonate solution (20 mL), pH=10 phosphate buffer (20 mL), and brine (20 mL), then dried on Na₂SO₄, filtered, concentrated and chromatographed on 5:1->1:5 Hx:EA, returning I.22 (70 mg, 0.14 mmol, 43%; R_{f}=0.24 & 0.12 on 1:1 Hx:EA, trans & cis isomers, FeCl₃) as a brown solid. ¹H-NMR (400 MHz): δ=8.87 (d, 2.4 Hz, 1H), 7.65 (dd, 8.6 & 2.4 Hz, 1H), 7.18 (s, 2H), 6.94 (d, 8.8 Hz, 1H), 5.61 (s br, 1H), 4.38-4.12 (m, 3H), 3.90 (s, 3H), 3.89 (s, 6H), 3.86 (s, 3H), 1.44 (s, 9H) ppm. ¹³C-NMR (100 MHz): δ=169.7, 157.4, 153.5 (×2), 150.5, 148.5, 146.7, 140.2, 127.7, 121.2, 112.9, 109.9, 100.2 (×2), 80.8, 62.7, 61.0, 56.2 (×2), 56.1, 50.1, 28.3 (×3) ppm. HRMS (ESI+) calcd for [C₂₄H₃₃N₄O₈]⁺═[MH]⁺: m/z 505.22929. found 505.22945. LCMS(+): tᵣₑₜ=3.52 & 4.22 min, each 505 Th=[MH]⁺, cis and trans isomers respectively.

### (2-methoxy-5-((3,4,5-trimethoxyphenyl)diazenyl)phenyl))-1-L-serinamide 2,2,2-trifluoroacetate salt (I.20)

To I.22 (55 mg, 0.11 mmol) were added DCM (1 mL) and TFA (1 mL) and the purple solution stirred at room temperature for 1 hr. The volatiles were removed at 1 mbar until a brown crude oil was obtained. Chromatography on 1:1:0->1:1:0.3 Hx:EA:MeOH returned unreacted I.22 (18 mg, 0.036 mmol) then I.20 2,2,2-trifluoroacetate salt (21 mg, 0.041 mmol, 37% or 55% brsm; R_{f}=0.35 on 1:1:0.3 Hx:EA:MeOH, FeCl₃) as a brown viscous oil. ¹H-NMR (400 MHz, DMSO): δ=8.63 (d, 2.5 Hz, 1H), 8.38 (s br, 3H), 7.80 (dd, 8.7 & 2.5 Hz, 1H), 7.32 (d, 8.9 Hz, 1H), 7.23 (s, 2H), 5.70 (s br, 1H), 4.70 (dd, 9.1 & 4.2 Hz, 1H), 4.54 (t, 8.9 Hz, 1H), 4.34 (dd, 8.8 & 4.1 Hz, 1H), 3.99 (s, 3H), 3.91 (s, 6H), 3.77 (s, 3H) ppm. ¹³C-NMR (100 MHz, DMSO): δ=172.0, 158.0 (q, 31.6 Hz), 153.4 (×2), 152.1, 147.8, 145.4, 139.9, 127.2, 122.5, 117.1 (q, 294 Hz), 113.7, 111.4, 99.9 (×2), 60.5, 60.2, 56.4, 56.0 (×2), 54.5 ppm. HRMS (ESI+) calcd for [C₁₉H₂₅N₄O₆]⁺═[MH]⁺: m/z 405.17686. found 405.17695. LCMS(+): tᵣₑₜ=2.30-2.65 min (broad), 405 Th═[MH]⁺.

### North Ring Ortho-Oxy-Linker-Bearing Derivatives (I.18) and (I.19)

The syntheses of I.18 and I.19 are presented on Scheme 18 hereafter.

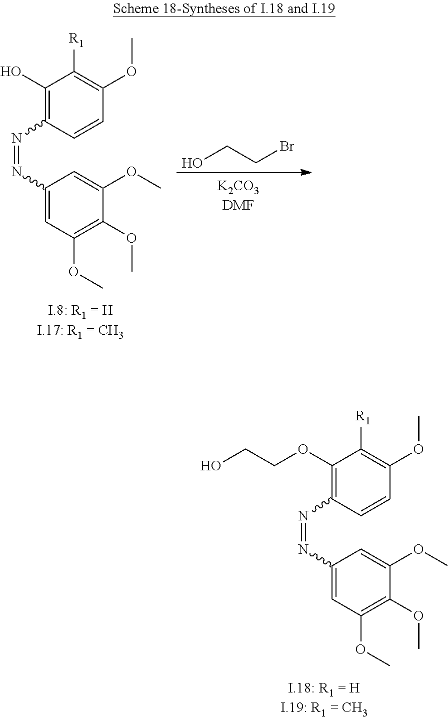

### 2-(3-methoxy-2-methyl-6-((3,4,5-trimethoxyphenyl)diazenyl)phenoxy)ethan-1-ol (I.18)

To I.8 (72 mg, 0.23 mmol) were added 2-bromoethanol (360 mg, 2.9 mmol), K₂CO₃ (400 mg, 2.8 mmol), and DMF (6 mL) and the mixture stirred overnight. Water (60 mL) was added and the aqueous phase extracted with EtOAc (2×15 mL); the combined organic layers were washed with water (10 mL), aqueous LiCl (10%, 5 mL), and brine (15 mL), then dried on Na₂SO₄, filtered and concentrated. After column chromatography on 5:1->1:1 Hx:EA, I.18 (44 mg, 0.12 mmol, 54%; R_{f}=0.18 on 1:1 Hx:EA, Han) was returned as a red oil. ¹H-NMR (400 MHz, CD₃CN): δ=7.67 (d, 9.0 Hz, 1H), 7.20 (s, 2H), 6.75 (d, 2.6 Hz, 1H), 6.64 (dd, 9.0 & 2.6 Hz, 1H), 4.27 (dd, 5.3 & 4.3 Hz, 2H), 3.92 (s, 6H), 3.91-3.87 (m overlapped, 2H), 3.88 (s, 3H), 3.82 (s, 3H) ppm. ¹³C-NMR (100 MHz, CD₃CN): δ=164.3, 158.9, 154.2 (×2), 149.6, 140.5, 137.3, 118.3, 107.8, 101.9, 100.5 (×2), 72.5, 61.0, 60.6, 56.3 (×2), 56.1 ppm. HRMS (ESI+) calcd for [C₁₈H₂₃N₂O₆]⁺═[MH]⁺: m/z 363.38945. found 363.15459. LCMS(+): tᵣₑₜ=3.12 & 3.85 min, each 363 Th═[MH]⁺; the first peak was assigned as the as isomer due to its absorbance shoulder centred at 445 nm.

### 2-(5-methoxy-2-((3,4,5-trimethoxyphenyl)diazenyl)phenoxy)ethan-1-ol (I.19)

To I.17 (150 mg, 0.45 mmol) were added 2-bromoethanol (220 mg, 1.8 mmol), K₂CO₃ (250 mg, 1.8 mmol), and DMF (5 mL) and the mixture stirred overnight. Water (20 mL) and aqueous KH₂PO₄ solution (10%, 5 mL) were added and the aqueous phase extracted with Et₂O (3×20 mL); the combined organic layers were washed with water (10 mL), aqueous LiCl (10%, 10 mL), and brine (10 mL), then dried on Na₂SO₄, filtered and concentrated. After column chromatography on 5:1->1:1 Hx:EA, 1.19 (114 mg, 0.30 mmol, 67%; R_{f}=0.72 on 1:5 Hx:EA, Van) was returned as a red oil. ¹H-NMR (400 MHz): δ=7.58 (dd, 9.1 Hz, 1H), 7.14 (s, 2H), 6.66 (d, 9.1 Hz, 1H), 4.22-4.16 (m, 2H), 3.89 (s, 6H), 3.85 (s, 3H), 3.84 (s, 4H), 3.84-3.80 (m, 2H), 2.18 (s, 3H) ppm. ¹³C-NMR (100 MHz): δ=161.7, 156.3, 153.6 (×2), 148.5, 140.4, 140.0, 120.7, 115.4, 106.5, 100.2 (×2), 61.7, 61.1, 56.3 (×2), 56.0, 9.3 ppm. HRMS (ESI+) calcd for [C₁₉H₂₅N₂O₆]⁺═[MH]⁺: m/z 377.17071. found 377.17016. LCMS(+): tᵣₑₜ=3.48 & 4.37 min, 377 Th=[MH]⁺; the first peak was assigned as the cis isomer due to its absorbance shoulder centred at 450 nm.

### (I.24)—a Phosphatase-Activated Prodrug of (I.1)

The syntheses of I.24 and its precursor IV.24 were carried out similarly to a described procedure^{[19]}, as is presented on Scheme 19 hereafter.

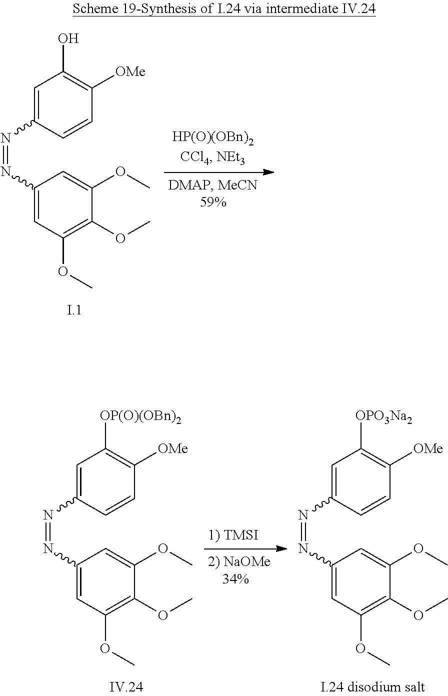

### dibenzyl (2-methoxy-5-((3,4,5-trimethoxyphenyl)diazenyl)phenyl) phosphate (IV.24)

Similarly to the described procedure^{[19]}, I.1 (100 mg, 0.31 mmol) was dissolved in dry acetonitrile (4 mL) under nitrogen, then the solution cooled to −30° C. CCl₄ (242 mg, 1.57 mmol) was added, then NEt₃ which had been stood on KOH (71 mg), and 4-(N,N-dimethylamino)pyridine (DMAP; 5 mg). Dibenzyl phosphite (122 mg, 0.46 mmol) was then added dropwise. The reaction was stirred for 3 h at −30° C., then as LCMS showed incomplete conversion of the starting material, additional dibenzyl phosphite (180 mg, 0.68 mmol) and CCl₄ (300 mg, 1.94 mmol) were added. The mixture was stirred warming to room temperature overnight. Aqueous KH₂PO₄ solution (10%, 10 mL) was added and the aqueous phase extracted with EtOAc (4×10 mL). The combined organic layers were washed with water (10 mL), brine (10 mL), dried on Na₂SO₄, filtered and concentrated. The crude oil thus obtained was chromatographed on 5:1->1:1 Hx:EA, giving IV.24 (105 mg, 0.18 mmol, 59%; R_{f}=0.38 and 0.22 on 1:1 Hx:EA (trans and cis isomers), Han) as a yellow oil. ¹H-NMR (400 MHz): δ=7.82 (dd, 2.3 & 1.5 Hz, 1H), 7.73 (ddd, 8.7 & 2.4 & 1.0 Hz, 1H), 7.33-7.21 (m, 10H), 7.14 (s, 2H), 6.96 (dd, 8.8 & 1.0 Hz, 1H), 5.14 (d, 7.9 Hz, 4H), 3.90 (s, 6H), 3.87 (s, 3H), 3.80 (s, 3H) ppm. ¹³C-NMR (100 MHz) showed some peaks split, perhaps for diastereotopicity around the phosphoester: δ=153.5 (×2), 153.1 & 153.0 (×1), 148.3, 146.3 & 146.3 (×1), 140.4, 140.2 & 140.1 (×1), 135.6 & 135.6 (×2), 128.6 (×4), 128.5 (×2), 127.9 (×4), 123.2 & 123.2 (×1), 114.2 & 114.2 (×1), 112.0, 100.3 (×2), 70.0 & 70.0 (×2), 61.1, 56.2 (ç2), 56.2 ppm. HRMS (ESI+) calcd for [C₃₀H₃₂N₂O₈P]⁺═[MH]⁺: m/z 579.18908. found 579.18938. LCMS(+): tᵣₑₜ=4.69 & 5.27 min, 579 Th═[MH]⁺; the first peak was assigned as the cis isomer due to its absorbance shoulder centred at 450 nm.

### 2-methoxy-5-((3,4,5-trimethoxyphenyl)diazenyl)phenyl phosphate disodium salt (I.24)

Similarly to a described, analogous procedure^{[19]}, to IV.24 (100 mg, 0.165 mmol) were added under nitrogen, NaI (49 mg, 0.33 mmol), dry acetonitrile (2.5 mL), and TMSCl (37 mg, 0.34 mmol). The mixture was stirred for 4 h at room temperature. Water (1.5 mL) and aqueous Na₂S₂O₃ solution (10%, 0.05 mL) were added and the aqueous phase extracted with EtOAc (3×10 mL). The combined organic layers were dried on Na₂SO₄, filtered and concentrated to a red crude oil. To the crude oil were added under nitrogen, dry MeOH (3 mL) and NaOMe (18.5 mg) and the reaction was stirred overnight. The volatiles were evaporated, and the yellow oily residue (principally containing 1.24 disodium salt together with its monobenzyl ester, which was identified by LCMS as the peak with tᵣₑₜ=3.08 min, 489 Th) was repeatedly triturated with cyclohexane (3×3 mL), 1:3 cyclohexane:ethyl acetate (5×2 mL), ethyl acetate (2×2 mL), and lastly acetone (2×2 mL), leaving 1.24 disodium salt as a yellow-brown powder (25 mg, 0.056 mmol, 34%) which was fully soluble in PBS to at least 25 mM. ¹H-NMR (400 MHz, D₂O): δ=8.33 (s, 1H), 7.63 (s, 1H), 7.39 (d, 8.7 Hz, 1H), 6.98 (d, 9.5 Hz, 1H), 6.97 (s, 2H), 3.80 (s, 3H), 3.77 (s, 6H), 3.70 (s, 3H) ppm. ¹³C-NMR (100 MHz, D₂O): δ=171.0, 153.4 & 153.3 (×1), 152.7, 148.2, 145.5, 142.5 & 142.4 (×1), 138.8, 128.5 & 127.6 (×1), 121.5, 112.1 (×2), 100.0 (×2), 60.9, 55.9 (×2), 55.9 ppm. HRMS (ESI−) calcd for [C₁₆H₁₈N₂O₈P]⁻═[M-H]⁻: m/z 397.08008. found 397.08029. LCMS(+): tᵣₑₜ=2.04 & 2.44 min, 399 Th=[MH]⁺; the first peak was assigned as the as isomer due to its absorbance shoulder centred at 450 nm.

### North Ring Ortho-Linked Fluorophore-Bearing Derivative (I.25)

The synthesis of I.25 is presented on Scheme 20 hereafter.

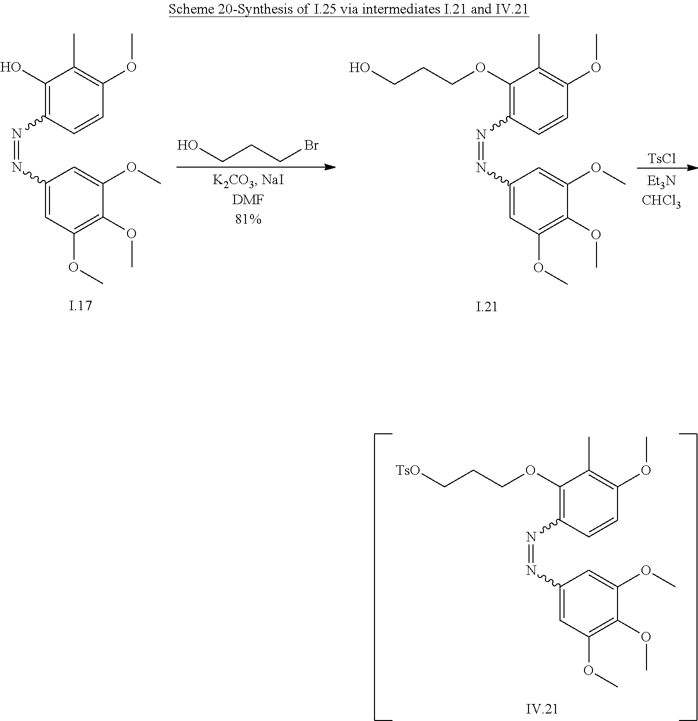

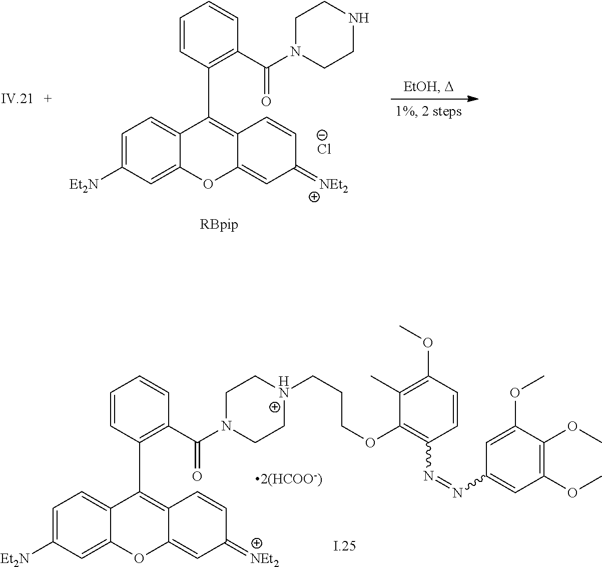

### 3-(3-methoxy-2-methyl-6-((3,4,5-trimethoxyphenyl)diazenyl)phenoxy)propan-1-ol (I.21)

To I.17 (165 mg, 0.50 mmol) were added NaI (15 mg, 0.1 mmol), 3-bromopropanol (210 mg, 1.53 mmol), K₂CO₃ (207 mg, 1.46 mmol), and DMF (6 mL) and the mixture stirred overnight at room temperature. Water (10 mL), aqueous LiCl (10%, 10 mL) and aqueous KH₂PO₄ solution (10%, 10 mL) were added and the aqueous phase extracted with CHCl₃ (10 mL) then Et₂O (2×15 mL); the combined organic layers were washed with water (10 mL), aqueous LiCl (10%, 10 mL), and brine (10 mL), then dried on Na₂SO₄, filtered and concentrated. After column chromatography on 5:1->1:1 Hx:EA, 1.21 (158 mg, 0.405 mmol, 81%; R_{f}=0.35 and 0.12 on 1:1 Hx:EA (trans and cis isomers), Van) was returned as a yellow oil. ¹H-NMR (400 MHz): δ=7.61 (d, 9.0 Hz, 1H), 7.22 (s, 2H), 6.66 (d, 9.1 Hz, 1H), 4.15 (t, 5.6 Hz, 2H), 3.91 (s, 6H), 3.91-3.77 (m, 2H), 3.86 (s, 3H), 3.84 (s, 3H), 2.18 (s, 3H), 2.03 1.98 (m, 2H) ppm. ¹³C-NMR (100 MHz): δ=161.5, 156.6, 153.6 (×2), 148.6, 140.3, 139.9, 120.6, 115.2, 106.4, 100.3 (×2), 75.0, 61.6, 61.1, 56.2 (×2), 55.9, 32.3, 8.96 ppm. HRMS (ESI+) calcd for [C₂₀H₂₇N₂O₆]⁺═[MH]⁺: m/z 391.18636. found 391.18604. LCMS(+): tᵣₑₜ=3.52 & 4.48 min, 391 Th=[MH]⁺; the first peak was assigned as the as isomer due to its absorbance shoulder centred at 450 nm.

### N-(6-(diethylamino)-9-(2-(4-(3-(3-methoxy-2-methyl-6-((3,4,5-trimethoxyphenyl)diazenyl)phenoxy)propyl)piperazine-1-carbonyl)phenyl)-3H-xanthen-3-ylidene)-N-ethylethanaminium bis(formate) salt (I.25)

Known compound N-(6-(diethylamino)-9-(2-(piperazine-1-carbonyl)phenyl)-3H-xanthen-3-ylidene)-N-ethylethanaminium chloride (abbreviated RBpip chloride) was made by the reported method and confirmed by NMR^{[46]}.

To I.21 (140 mg, 0.36 mmol) were added CH₂Cl₂ (5 mL), Et₃N (89 mg, 0.88 mmol), 4-dimethylaminopyridine (10 mg), and TsCl (69 mg, 0.36 mmol), and the mixture stirred at room temperature for 4 hours until TLC indicated complete conversion of the starting material, presumably to the tosylate IV.21. After evaporation of the volatiles, to the residue were added EtOH (15 mL), NaI (20 mg), RBpip chloride (160 mg, 0.29 mmol) and NEt₃ (81 mg), and the mixture stirred at 80° C. for 2 days under closed air atmosphere. After cooling and evaporation of the volatiles, the crude residue was chromatographed on a small volume of silica gel to separate the bulk of the impurities, using a gradient of 1:1:0:0->1:1:1:0->1:1:0:0->1:1:0:1->0:0:0:1->0:0:1:1 Hx:EA:MeOH:CH₂Cl₂. The third red fraction to elute contained the pink-fluorescent product cation of 1.25 (R_{f}=0.0 on 1:1:0.2 Hx:EA:MeOH, 0.25<R_{f}<0.4 on 9:1 DCM:MeOH) as well as substantial impurities. This fraction was then concentrated (dry weight: 45 mg) then separated by semi-preparative HPLC on reverse-phase column with a 10:90->60:40 MeCN:water eluent gradient (water component contains 0.1% formic acid), and pure I.25 as the bis(formate) salt (2.0 mg, 2.0 μmol, 1%) was recovered from the pure fractions as a dark purple solid. ¹H-NMR (400 MHz, CD₃CN) showed overlapping peaks from two components at roughly 5:2 ratio (possibly conformers about the piperazine moiety, as suggested by the ¹³C-NMR spectrum): δ=8.37 (s, 2H), 7.74-7.70 (m, 2H), 7.63-7.58 (m, 2H), 7.47-7.42 (m, 1H), 7.23 (s, 2H), 7.19 (s, 1H), 7.00-6.94 (m, 2H), 6.87-6.82 (m, 4H), 4.16 (t, 6.3 Hz, 2H), 3.91 (s, 3H), 3.90 (s, 6H), 3.82 (s, 3H), 3.66-3.58 (m, 11H), 3.36-3.24 (m, 5H), 2.47-2.40 (m, 2H), 2.20 (s, 3H), 2.01-1.96 (m, 2H), 1.25 (t, 7.1 Hz, 12H) ppm. ¹³C-NMR (100 MHz): δ=167.2, 165.0 (×2), 161.8, 158.3 (×2), 157.5, 156.7, 156.2 (×2), 154.3 (×2), 149.6, 140.7, 140.2, 136.6, 132.7 (×2), 131.2, 130.5, 130.4, 130.0, 128.0, 120.5, 114.8 (×2), 114.6 (×2), 114.2, 106.8, 100.6 (×2), 96.4 (×2), 74.6, 60.6, 56.4 (×2), 56.3, 55.2, 53.3, 52.9, 48.8, 47.7, 46.2 (×4), 27.9, 12.4 (×4), 8.9 ppm. LCMS(+): tᵣₑₜ=3.25 & 3.45 min, each 883 Th═[MH]⁺: peaks assigned as cis & trans isomers respectively since the second peak has substantially greater absorbance at 390 nm. HRMS (ESI+) calcd for [C₅₂H₆₃N₆O₇]⁺═[M]⁺: m/z 883.47582. found 883.47453.

### Meta-Hydro, Ortho-Anilide Derivative (I.26)

The synthesis of I.26 is presented on Scheme 21 hereafter.

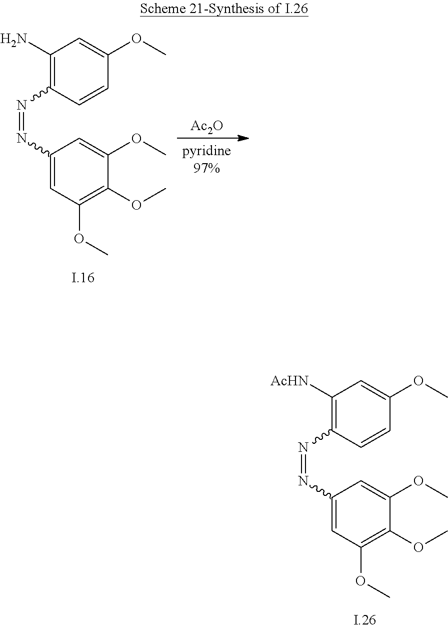 [SMILES: "Ac2O" ; COc1ccc(N=Nc2cc(OC)c(OC)c(OC)c2)c(N)c1 ; COc1ccc(N=Nc2cc(OC)c(OC)c(OC)c2)c(NC(C)=O)c1]

### N-(5-methoxy-2-((3,4,5-trimethoxyphenyl)diazenyl)phenyl)acetamide (I.26)

To I.16 (51 mg, 0.16 mmol) were added pyridine (5 mL) and acetic anhydride (0.5 mL) and the mixture stirred overnight. After evaporation of the volatiles at 2 mbar and 30° C., the residue was partitioned between aqueous HCl (1 M, 5 mL) and EtOAc (5 mL), then the aqueous layer was extracted with EtOAc (2×10 mL); the combined organic layers were washed with aqueous HCl (1 M, 5 mL) and brine (5 mL), then dried on Na₂SO₄, filtered and concentrated to an olive powder which was spectroscopically pure I.26 (56 mg, 0.16 mmol, 97%; R_{f}=0.15 on 2.4:1 Hx:EA (trans and as isomers overlapped), FeCl₃). ¹H-NMR (400 MHz): δ=10.53 (s, 1H), 8.24 (d, 2.7 Hz, 1H), 7.76 (d, 9.0 Hz, 1H), 7.04 (s, 2H), 6.65 (dd, 9.0 & 2.8 Hz, 1H), 3.89 (s, 6H), 3.87 (s, 3H), 3.84 (s, 3H), 2.20 (s, 3H) ppm. ¹³C-NMR (100 MHz): δ=168.8, 163.3, 153.7 (×2), 148.2, 140.4, 137.1, 133.1, 124.9, 110.8, 103.7, 99.7 (×2), 61.2, 56.2 (×2), 55.8, 25.6 ppm. HRMS (ESI−) calcd for [C₁₈H₂₀N₃O₅]⁻═[M-H]⁻: m/z 358.14030. found 358.14059. LCMS(+): tᵣₑₜ=3.12 & 4.41 min, each 360 Th═[MH]⁺: these peaks were assigned to the cis & trans isomers respectively since the UV absorption profile of the first peak (cis) featured a shoulder centred around 450 nm which was absent in the second peak.

### Meta-Bromo Derivative (I.27)

The synthesis of I.27 is presented on Scheme 22 hereafter.

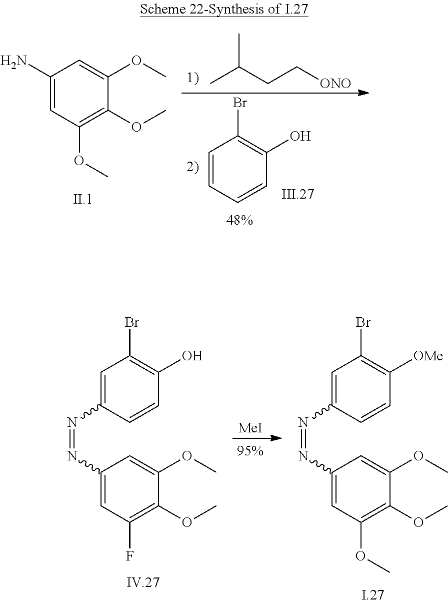

### 2-bromo-4-((3,4,5-trimethoxyphenyl)diazenyl)phenol (IV.27)

By Standard Procedure A, II.1 (3.67 g, 20.0 mmol) was reacted with commercial 2-bromophenol (III.27; 3.46 g, 20.0 mmol). Chromatography of the orange crude oil on 5:1->1:1 Hx:EA returned IV.27 (3.52 g, 9.62 mmol, 48%; R_{f}=0.66 on 1:1 Hx:EA, Han) as a yellow solid. ¹H-NMR (400 MHz): δ=8.03 (d, 2.3 Hz, 1H), 7.80 (dd, 8.7 & 2.3 Hz, 1H), 7.15 (s, 2H), 7.09 (d, 8.7 Hz, 1H), 3.89 (s, 6H), 3.87 (s, 3H) ppm. ¹³C-NMR (100 MHz): δ=154.4, 153.5 (×2), 148.2, 147.1, 140.6, 125.5, 125.5, 116.1, 111.1, 100.3 (×2), 61.1, 56.2 (×2) ppm. HRMS (ESI+) calcd for [C₁₅H₁₆N₂O₄Br]⁺═[MH]⁺: m/z 367.02880. found 367.02857. LCMS(+): tᵣₑₜ=4.38 min, 367 and 369 Th=[MH]⁺.

### 1-(3-bromo-4-methoxyphenyl)-2-(3,4,5-trimethoxyphenyl)diazene (I.27)

By Standard Procedure B, IV.27 (2.40 g, 6.54 mmol) was methylated overnight. Chromatography on 5:1->1:1 Hx:EA returned I.27 (2.37 g, 6.21 mmol, 95%; R_{f}=0.69 and 0.51 on 1:1 Hx:EA, trans and as isomers, FeCl₃) as orange crystals. ¹H-NMR (400 MHz): δ=8.19 (d, 2.3 Hz, 1H), 7.94 (dd, 8.7 & 2.4 Hz, 1H), 7.24 (s, 2H), 7.05 (d, 8.8 Hz, 1H), 4.01 (s, 3H), 3.99 (s, 6H), 3.96 (s, 3H ppm. ¹³C-NMR (100 MHz): δ=157.8, 153.5 (×2), 148.3, 146.9, 140.5, 126.0, 125.6, 112.7, 111.4, 100.3 (×2), 61.1, 56.6, 56.2 (×2) ppm. LCMS(+): tᵣₑₜ=4.20 & 5.28 min, each 381 and 383 Th═[MH]⁺: these peaks were assigned to the cis & trans isomers respectively since the UV absorption profile of the first peak (cis) featured a shoulder centred around 445 nm which was absent in the second peak. HRMS (ESI+) calcd for [C₁₆H₁₈H₂O₄Br]⁺═[MH]⁺: m/z 381.04445. found 381.04419.

### Meta-Electrophile Derivatives (I.28) and (I.29)

Compound (I.27) could be used as a convenient starting point for divergent synthesis of a variety of meta-substituted polar derivatives, via lithium-halogen exchange followed by a range of electrophilic quenches. The syntheses of I.28 and I.29 are presented on Scheme 23 hereafter.

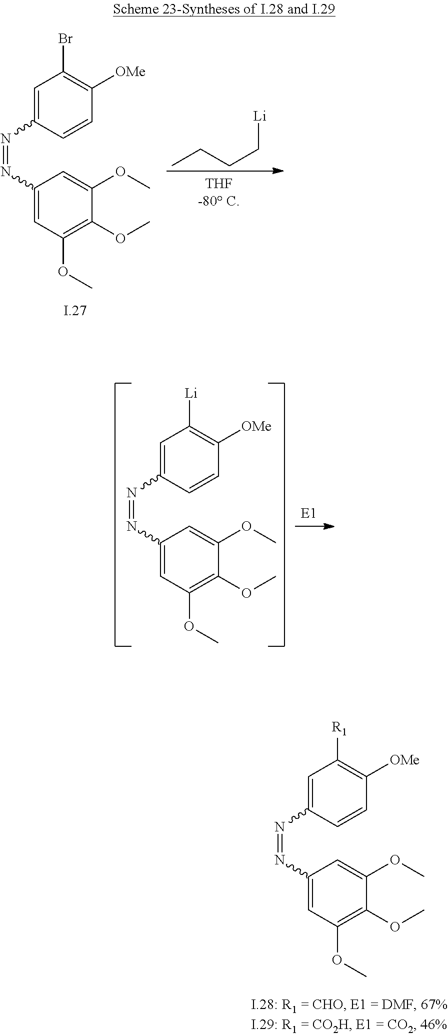

### (2-methoxy-5-((3,4,5-trimethoxyphenyl)diazenyl)phenyl)lithium stock solution “ALi”

To I.27 (1.20 g, 3.15 mmol) under nitrogen at −80° C. were added dry THF (9 mL) and, dropwise, n-butyllithium (2.5 M in hexanes, 1.32 mL, 3.30 mmol). The solution darkened significantly over the course of the addition. This stock solution “ALi” of the azoaryllithium intermediate (approximately 0.30 M) was aged at −80° C. for 1 hour, then used without further delay.

### 2-methoxy-5-((3,4,5-trimethoxyphenyl)diazenyl)benzaldehyde (I.28)

To a solution of DMF (0.2 mL) in dry THF (3 mL) at −80° C. under nitrogen was added dropwise the stock solution “ALi” (1.0 mL, approx. 0.30 mmol). The solution was warmed to room temperature and stirred for 30 min, then quenched by its dropwise addition into a rapidly-stirred mixture of aqueous KH₂PO₄ solution (10%, 15 mL) and Et₂O (10 mL). The aqueous layer was extracted with Et₂O (2×10 mL), then the combined organic layers were washed with water (10 mL), brine (10 mL), dried on Na₂SO₄, filtered and concentrated. The brown crude oil was separated by chromatography on 5:1->1:1 Hx:EA gradient yielding 1.28 (67 mg, 0.21 mmol, 67%; R_{f}=0.50 and 0.29 on 1:1 Hx:EA, trans and cis isomers, Han) as a yellow oil. ¹H-NMR (400 MHz): δ=10.45 (s, 1H), 8.34 (d, 2.6 Hz, 1H), 8.08 (dd, 8.9 & 2.6 Hz, 1H), 7.17 (s, 2H), 7.06 (d, 8.9 Hz, 1H), 3.96 (s, 3H), 3.90 (s, 6H), 3.86 (s, 3H) ppm. ¹³C-NMR (100 MHz): δ=189.4, 163.3, 153.5 (×2), 148.3, 146.3, 140.6, 130.0, 125.0, 123.4, 112.1, 100.3 (×2), 61.1, 56.2 (×3) ppm. HRMS (ESI+) calcd for [C₁₇H₁₉N₂O₅]⁺═[MH]⁺: m/z 331.12885. found 331.12855. LCMS(+): tᵣₑₜ=3.61 & 4.59 min, each 331 Th═[MH]⁺: these peaks were assigned to the cis & trans isomers respectively since the UV absorption profile of the first peak (cis) featured a shoulder centred around 440 nm which was absent in the second peak.

### 2-methoxy-5-((3,4,5-trimethoxyphenyl)diazenyl)benzoic acid (I.29)

To a mixture of solid CO₂ (1 g) in dry THF (5 mL) at −80° C. under nitrogen was added dropwise the stock solution “ALi” (1.0 mL, approx. 0.30 mmol). The drops of “ALi” lightened in colour immediately upon leaving the needle. The resultant yellow solution was warmed to room temperature and stirred for 5 min, then poured into a rapidly-stirred mixture of aqueous KH₂PO₄ solution (10%, 15 mL) and Et₂O (10 mL), rinsing the flask once with Et₂O (5 mL). The aqueous layer was extracted with Et₂O (2×10 mL), then the combined organic layers were washed with water (10 mL), brine (10 mL), dried on Na₂SO₄, filtered and concentrated. The brown crude oil was separated by chromatography on 5:1:0->1:1:0->1:1:0.5 Hx:EA:MeOH gradient yielding 1.29 (48 mg, 0.14 mmol, 46%; R_{f}=0.13 on 1:1:0.5 Hx:EA:MeOH, trans and as isomers overlap, Han) as a brown solid. ¹H-NMR (400 MHz, CD₃CN): δ=8.46 (d, 2.6 Hz, 1H), 8.13 (dd, 8.9 & 2.6 Hz, 1H), 7.35 (d, 8.9 Hz, 1H), 7.30 (s, 2H), 4.09 (s, 3H), 3.93 (s, 6H), 3.83 (s, 3H) ppm. ¹³C-NMR (100 MHz): δ=165.6, 159.7, 153.6 (×2), 148.3, 147.0, 140.9, 129.0, 128.6, 118.4, 112.3, 100.5 (×2), 61.2, 57.2, 56.3 (×2) ppm. HRMS (ESI+) calcd for [C₁₇H₁₉N₂O₆]⁺═[MH]⁺: m/z 347.12376. found 347.12343. LCMS(+): tᵣₑₜ=2.95 & 3.69 min, each 347 Th═[MH]⁺: these peaks were assigned to the cis & trans isomers respectively since the UV absorption profile of the first peak (cis) featured a shoulder centred around 440 nm which was absent in the second peak.

### Para-Trifluoromethoxy Derivative (I.30) Formed by Mills Reaction

The synthesis of I.30 is presented on Scheme 24 hereafter.

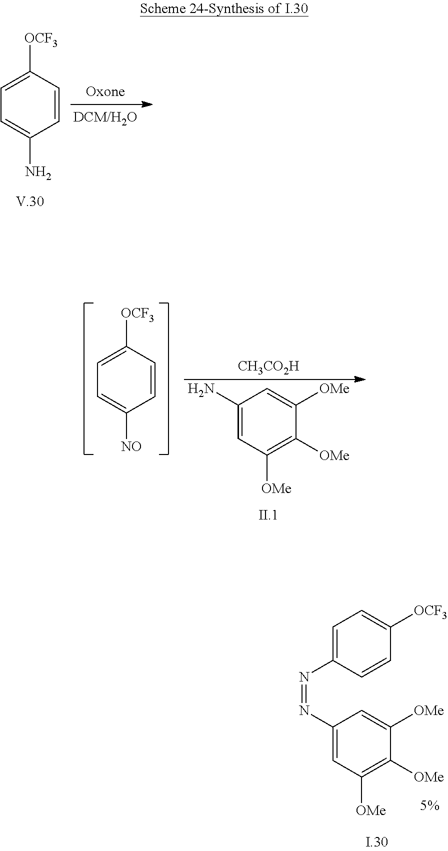

### 1-(4-(trifluoromethoxy)phenyl)-2-(3,4,5-trimethoxyphenyl)diazene (I.30)

To commercial 4-(trifluoromethoxy)aniline V.30 (250 mg, 1.41 mmol) were added DCM (10 mL) and water (10 mL). Oxone® (867 mg, 2.83 mmol) was added and the mixture was stirred vigorously at room temperature for 16 hours. The organic layer was separated, washed with aqueous HCl (1M, 2×10 mL) and brine (10 mL), dried on Na₂SO₄, filtered and concentrated quickly to 40 mbar at 40° C. on the rotavap. Glacial acetic acid (6 mL) and II.1 (256 mg, 1.40 mmol) were added and the mixture stirred at 60° C. for 6 hours. The reaction was neutralised by pouring into a saturated solution of NaHCO₃ and K₂CO₃ (50 mL), then extracted with EtOAc (2×20 mL). The combined organic layers were washed with brine (10 mL), dried on Na₂SO₄, filtered and concentrated. The crude residue was separated by chromatography with 10:1->2.4:1 Hx:EA gradient, giving I.30 (25 mg, 0.070 mmol, 5%; R_{f}=0.75 and 0.58 on 2.4:1 Hx:EA, Han) as a red solid. ¹H-NMR (400 MHz): δ=7.87 (˜d, 8.9 Hz, 2H), 7.28 (d, 8.0 Hz, 1H), 7.18 (s, 2H), 3.90 (s, 6H), 3.87 (s, 3H) ppm. ¹³C-NMR (100 MHz): δ=153.6 (×2), 150.7, 150.7, 148.3, 141.0, 124.2 (×2), 121.3 (×2), 120.4 (q, 258 Hz), 100.5 (×2), 61.1, 56.2 (×2) ppm. HRMS (ESI+) calcd for [C₁₆H₁₆N₂O₄F₃]⁺═[MH]⁺: m/z 357.10567. found 357.1054. LCMS(+): tᵣₑₜ=4.53 and 5.41 min, 357 Th=[MH]⁺: these peaks were assigned to the cis & trans isomers respectively since the UV absorption profile of the first peak (cis) featured a shoulder centred around 435 nm which was absent in the second peak.

### Prior Art Hydro-Derivative (I.6)

Compound I.6^{[13,14]} was also synthesized, and its synthesis (presented on Scheme 25 hereafter) and properties are therefore reported.

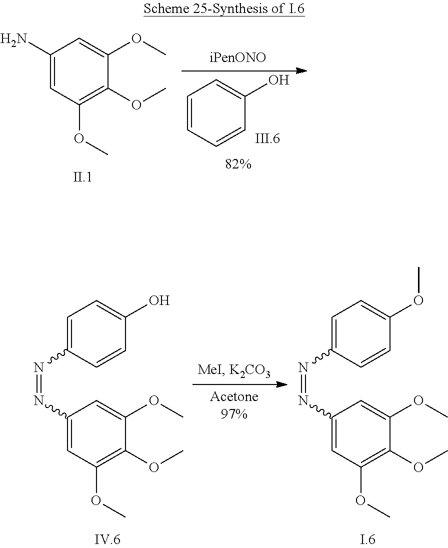

### 4-((3,4,5-trimethoxyphenyl)diazenyl)phenol (IV.6)^{[13]}

By Standard Procedure A, II.1 (174 mg, 0.95 mmol) was reacted with commercial phenol (III.6; 102 mg, 1.08 mmol). Chromatography of the red crude oil on 5:1->2.4:1 Hx:EA returned IV.6 (227 mg, 0.78 mmol, 82%; R_{f}=0.64 on 1:1 Hx:EA, FeCl₃) as a red oil. ¹H-NMR (400 MHz): δ=7.81 (d, 8.8 Hz, 2H), 7.19 (s, 2H), 6.89 (d, 8.9 Hz, 2H), 3.89 (s, 6H), 3.86 (s, 3H) ppm. ¹³C-NMR (100 MHz): δ=159.1, 153.5 (×2), 148.2, 146.6, 140.2, 125.1 (×2), 116.0 (×2), 100.1 (×2), 61.1, 56.2 (×2) ppm. HRMS (ESI+) calcd for [C₁₅H₁₇N₂O₄]⁺═[MH]⁺: m/z 289.11828. found 289.11813.

### 1-(4-methoxyphenyl)-2-(3,4,5-trimethoxyphenyl)diazene (I.6)^{[13]}

By Standard Procedure B, IV.6 (226 mg, 0.77 mmol) was methylated overnight. Chromatography on 5:1->3:1 Hx:EA returned I.6 (231 mg, 0.76 mmol, 97%; R_{f}=0.76 and 0.60 on 1:1 Hx:EA, trans and cis isomers, FeCl₃) as a red oil. ¹H-NMR (400 MHz): δ=7.85 (˜d, 8.9 Hz, 2H), 7.16 (s, 2H), 6.95 (d, 9.0 Hz, 2H), 3.90 (s, 6H), 3.86 (s, 3H), 3.83 (s, 3H) ppm. ¹³C-NMR (100 MHz): δ=162.0, 153.5 (×2), 148.5, 146.7, 140.2, 124.7 (×2), 114.3 (×2), 100.1 (×2), 61.1, 56.2 (×2), 55.6 ppm. LCMS(+): tᵣₑₜ=3.80 & 4.78 min, each 303 Th═[MH]⁺: these peaks were assigned to the cis & trans isomers respectively since the UV absorption profile of the first peak (cis) featured a shoulder centred around 440 nm which was absent in the second peak. HRMS (ESI+) calcd for [C₁₆H₁₉N₂O₄]⁺═[MH]⁺: m/z 303.13393. found 303.13371.

### EXAMPLES PART B

### Photocharacterisation In Vitro

### Rationale for In Vitro Studies:

The purpose of these in vitro photocharacterisations was to analyse and compare qualitatively the behaviour of the synthesized azoaryls according to the invention, as regards important factors for their photoisomerisation, under conditions which could be easily measured, and which could most easily and generally be translated into qualitatively correct behaviour in cellulo and hopefully in vivo. Therefore favoured measurement conditions were taken at 37° C. and in aqueous solution at pH˜7 containing a minimum of cosolvent. As discussed in the literature^{[7]}, three key isomerisation parameters for sophisticated biological applications were: (1) PSS(λ), the fraction of as isomer established in a sample at the photostationary state (when the trans<->cis photoisomerisations are in equilibrium under saturating photon flux) as a function of wavelength. PSS(λ) depends strongly on the relative ratio between ε_{E}(λ) and ε_{Z}(λ) (the absorption coefficients of the trans and cis forms at wavelength λ), among other factors; (2) the relative efficiency E(λ) of approaching the PSS(λ) as a function of the applied wavelength λ. E(λ) reflects the magnitude of the photon flux one would need to apply to photoisomerise a mixture of trans and cis forms from a starting cis/trans ratio by a given percentage towards the cis/trans ratio at PSS(λ). E(λ) depends strongly on the absolute magnitudes of both ε_{E}(λ) and ε_{Z}(λ), among other factors; (3) the thermal reversion halflife τ for the spontaneous cis->trans isomerisation.

PSS(λ), E(λ) and τ inform the design of lighting conditions for realistic long-term biological experiments where samples should not be irradiated at high intensities or with high net flux^{[10,47]}. To illustrate: a typical biological study examining light-dependent inhibition of tubulin polymerisation (See Part D for details) incubated cells with a known concentration of azoaryl according to the invention, maintaining a negative control group in the dark, while irradiating active groups using light at wavelengths (λ), each with relatively narrow bandwidth such as ±15 nm. Irradiation could be applied in pulses, with the interval tₚₐᵤₛₑ between pulses chosen to be significantly less than τ. Then, knowing I(λ) (the relative intensity of the light source as a function of wavelength), 1/[I(λ)×E(λ)] could be used as a scaling factor to determine the relative pulse durations to apply as a function of the chosen wavelengths, thus ensuring that the PSS was approximately reached at each wavelength applied. If the PSS(λ) were known, an estimation of [Z]* (the time-average concentration of cis-azoaryl present during the assay) could then be possible for each wavelength applied. The local net antitubulin or cytotoxic effect generated in the assay protocol could then be understood simplistically as the product of [Z]* and a factor which would describe the cis-azoaryl's strength of tubulin polymerisation inhibition or cytotoxicity. Therefore it was considered important to determine at least some estimates for PSS(λ), E(λ) and τ which could preferably be intercomparable between compounds, in order to design and analyse biological experimental data later.

Sufficiently determining ε_{E}(λ) and ε_{Z}(λ), PSS(λ), E(λ) and τ:

A(λₘₑₐₛ, λ_{irrad}) is used to indicate the measured UV-Vis absorption spectrum in the range 340 nm<λₘₑₐₛ<650 nm, as a function of the irradiating wavelength λ_{irrad} once PSS(λ_{irrad}) has been established. UV-Vis measurement of A(λₘₑₐₛ, λ_{irrad}) was performed. It was considered that the PSS had been established when the absorbance profile ceased to alter under continued irradiation. An isosbestic point (wavelength λᵢₛₒ) was found for each compound by examining where the absorption A(λₘₑₐₛ) was invariant with respect to changing λ_{irrad}. A(λₘₑₐₛ, dark) was also acquired with samples that had been kept in the dark for at least several hours, and it was assumed that no significant spectral contribution from the cis isomer was present in these spectra. Standard cell culture purpose phosphate buffer saline at pH˜7.4 (PBS) with a fixed percentage of cosolvent, at 37° C., under air atmosphere, was used throughout.

HPLC (high-performance liquid chromatography) was then used as described in Part A to measure ε*_{E}(λ) and ε*_{Z}(λ) (the separated absorption profiles of trans and cis isomers respectively, in arbitrary units which are not intercomparable in an absolute sense). It was approximated that these spectra would be identical in shape if measured under the UV-Vis conditions, so once ε*_{E}(λ) and ε*_{Z}(λ) were scaled appropriately relative to each other, these scaled spectra could be used to deconvolute A(λₘₑₐₛ, λ_{irrad}). This approximation was considered useful especially in the regions of greatest spectral interest, ie. regions of relatively strong absorption (since only rather well-absorbed wavelengths will allow significant photoisomerisation under finite photon flux). To place the arbitrary-unit absorption spectra on an absolute scale, the maximal absorption wavelength λ_{strong} of the trans-isomer was first selected by examining A(λₘₑₐₛ, dark): typically, λ_{strong}≈380 nm. with Expressing A(λ_{strong}, dark) in units M⁻¹cm⁻¹, ε*_{E}(λ_{strong}) was then scaled so that ε_{E}(λ_{strong})=A(λ_{strong}, dark), giving:

ε_{E}(λ)=ε*_{E}(λ)×[A(λ_{strong},dark)/ε*_{E}(λ_{strong)]}

and the isosbestic point was used to scale the cis-isomer spectrum as per:

ε_{Z}(λ)=ε*_{Z}(λ)×[ε_{E}(λᵢₛₒ)/ε*_{Z}(λᵢₛₒ)]

A(λᵢₛₒ) could also be used to scale the spectra, with the advantage that finding A(λᵢₛₒ) does not require samples to be kept in the dark prior to UV-Vis measurement. However, using A(λ_{strong}, dark) was preferred, since A(λᵢₛₒ) was typically so much smaller that the standard deviation in the scaled absorptivities which it generated was far greater. It should be noted that certain compounds displayed a rather strong dependency of absorption spectrum upon the pH, possibly connected to their protonation state; and some compounds were markedly solvatochromic; therefore ε_{E}(λ) and ε_{Z}(λ) are considered only as reasonable approximations to the absorptivities that could be expected under diverse biological conditions.

In a simple analysis of the PSS(λ) and E(λ), it was approximated that Φ_{EZ} and Φ_{ZE} (the quantum efficiencies of the photoisomerisations trans->cis and cis->trans, respectively) are equal and independent of wavelength, and also that the kinetics of spontaneous reversion could be ignored (eg. high-intensity light source). A function φ(λ) was then calculated as per:

φ(λ)=ε_{E}(λ)/[ε_{Z}(λ)+ε_{E}(λ)]

If Φ_{EZ} and Φ_{ZE} are equal and independent of wavelength, φ(λ) gives the true PSS(λ). The wavelength independence of Φ_{EZ} and Φ_{ZE} may be an acceptable approximation in regions of relatively strong absorption within the visible spectrum. Their equality may also be an acceptable assumption: since if Φ_{EZ} and Φ_{ZE} are unequal but only depend weakly on wavelength in regions of relatively strong absorption, then φ(λ) is a transform of the true PSS(λ); this transform preserves the features of PSS(λ) which were most desired for evaluation in this study as long as Φ_{EZ} and Φ_{ZE} are not too different (eg less than a factor of ten difference). Indeed, the calculated φ(λ) were later confirmed by in cellulo experiments to give reliable indications of favourable wavelengths for bulk trans->cis and bulk cis->trans photoisomerisation, which is the benchmark for a suitable analysis of the PSS(λ). The advantages of calculating φ(λ) rather than exhaustively measuring the PSS(λ) experimentally are that (a) the throughput is much faster, (b) the true PSS(λ) may be difficult to achieve at weakly-absorbing wavelengths or with fast-relaxing compounds, (c) the true PSS(λ) may be difficult to measure accurately at wavelengths where one isomer strongly dominates the sample even if both are relatively strongly absorbing, and (d) the variation of PSS(λ) with changes in the local biological microenvironment (polarity, pH etc) may anyway be larger than the error incurred by the assumption φ(λ)═PSS(λ).

Also following the assumption that Φ_{EZ} and Φ_{ZE} are equal and independent of wavelength, a photoisomerisation efficiency E(λ) was calculated as per:

E(λ)=[ε_{E}(λ)+ε_{Z}(λ)]/[ε_{E}(390)+ε_{Z}(390)]

Larger values of E(λ) therefore denote higher efficiency photoisomerisation (less photon flux is required to approach the PSS(λ)). E(λ) was parametrised to the absorption coefficients at 390 nm, since typically that wavelength gave satisfactorily strong absorption and efficient photoisomerisation. It should be noted that E(λ) are not intercomparable between different compounds. This reflects the realistic scenario that the Φ_{EZ} (or the Φ_{ZE}) of two arbitrarily chosen compounds may differ by a significant amount, even if the approximation that Φ_{EZ} and Φ_{ZE} are equal and independent of wavelength applies independently to each compound.

Finally, trends in τ were ascertained by establishing the PSS at λ_{strong}, then turning off the light source at time t₀, and measuring A(λ_{strong}, t) over time. Data were acquired for half an hour, or else the first three halflives (whichever was the shorter period), then fitted to the equation:

A(λ_{strong},t)=A(λ_{strong},dark)−[A(λ_{strong},dark)−A(λ_{strong},t₀)]×e^{-kt}

This fit was used to return an in vitro halflife τ* for the cis->trans spontaneous thermal reversion isomerisation process, defined as τ=ln 2/k. It should be noted however, that azoaryl compounds' thermal reversion rates are known to depend strongly upon their microenvironment, such as pH and solvent/solvation effects^{[7,38]}. This was shown for compound (I.1), where a change of only 1.4 pH units gave a 25% change in τ*; note that different compounds are expected to respond very differently to changes in pH so no unifying trends are expected. Therefore the τ* acquired in vitro were considered appropriate only for qualitative intercomparison between samples (trend of τ* vs structure), rather than quantitative prediction of the τ values which might be experienced in cellulo or in vivo. Indeed, these in vitro τ* values appeared at least three times longer than what would have been expected based on in cellulo experiments where the pause time between activating pulses was varied.

### Spectrophotometry Equipment:

Absorption spectra in cuvette (“UV-Vis”) were acquired on a Varian CaryScan 50 (1 cm, 100 μL or 1 mL volume) with Peltier cell temperature control unit maintained at 37° C., in PBS at pH˜7.4 containing a low percentage of cosolvent if needed (typically 2% MeCN or 5% DMSO). A ThorLabs Polychrome V monochromator with a fibre optic cable output directed into the cuvette was used to perform photoisomerisation studies by UV-Vis spectrophotometry although single LEDs of approximately 10-20 mW output, 15-20° cone angle, and 10-15 nm bandwidth FWHM, obtained commercially from Roithner Lasertechnik GmbH, were equally successful in providing repeatable monochromatic photoisomerisation. Separated spectra of trans and as forms were acquired from the inline Diode Array Detector on the AGILENT 1260 SL coupled LC-MS system after HPLC separation. Fluorescence spectrophotometry (excitation and emission spectra) were acquired on a Varian CaryEclipse fluorescence spectrophotometer.

### Photocharacterisation Results 1: Trans- and Cis-Absorption Spectra

By the above procedure, the trans- and cis-isomers of each species were separated by HPLC as described in Part A, and ε*_{E}(λ) and ε*_{Z}(λ) were measured on the inline ultraviolet/visible Diode Array Detector; these were then scaled as outlined above. The parameters λ_{strong}, ε_{E}(λ_{strong}) and λᵢₛₒ are tabulated for selected compounds below (Table 1).

TABLE 1
λ_{strong}, ε_{E}(λ_{strong}) and λᵢₛₒ for selected compounds, measured in
PBS, at pH = 7.4 unless indicated otherwise, containing 20% MeCN
to ensure solubility. “nd” indicates “not determined”, ie. no clear
isosbestic point could be established due to insufficient measurable
photoisomerisation. Compound I.25 displayed a bimodal absorbance
spectrum, mainly attributable to the azoaryl moiety in the spectral
range 360-450 nm, and mainly attributable to the fluorophore
moiety above 510 nm, hence values are given for each range;
“510 ff” indicates that A(λ > 510) was invariant upon
trans<->cis photoswitching.

| | λ_{strong} | ε_{E}(λ_{strong}) | |
|---|---|---|---|
| Compound | (nm) | (M⁻¹cm⁻¹) | λᵢₛₒ (nm) |
| I.1 | 375 | 17700 | 470 |
| I.1, pH = 6 | 380 | 17100 | 465 |
| I.2 | 365 | 11400 | 470 |
| I.3 | 365 | 13300 | 445 |
| I.4 | 365 | 19804 | 455 |
| I.5 | 365 | 19578 | 450 |
| I.6 | 365 | 16300 | 445 |
| I.7 | 365 | 13600 | 455 |
| I.8 | 390 | 14900 | nd |
| I.9 | 355 | 11400 | 430 |
| I.11 | 375 | 18163 | 460 |
| I.12 | 380 | 12400 | 485 |
| I.13 | 355 | 14200 | 440 |
| I.14 | 390 | 18000 | 485 |
| I.16 | 410 | 14430 | nd |
| I.17 | 385 | 10872 | nd |
| I.25 | 375 | 15300 | 460 |
| | (565) | (66600) | (510 ff) |
| I.26 | 380 | 16300 | 475 |
| I.27 | 365 | 19540 | 445 |
| I.28 | 360 | 17813 | 452 |
| I.29 | 365 | 20102 | 447 |

The complete absorbance spectra of selected compounds are presented in FIG. 1.

Table 1 and FIG. 1 illustrate the large single-photon absorption coefficients within the in vivo compatible wavelength range which are typical for the compounds of the invention, and which especially distinguish them from stilbenes. Such strong absorption coefficients, coupled with the high quantum efficiencies known to be typical of azobenzene photoisomerisation^{[7,32]}, enable efficient single-photon photoisomerisation of the compounds of the invention in both directions cis->trans and trans->cis, with low power irradiation as is in vivo compatible, cheap and practical.

### Photocharacterisation Results 2: Calculated φ(λ) and E(λ)

φ(λ) and E(λ) were calculated as described above and typical examples are presented in FIG. 2.

FIG. 2 indicates that structural modifications within the scope of the compounds of the invention may substantially alter both the proportion of the as form in the photostationary state at different wavelengths, and the relative efficiency of approaching those photostationary states. This illustrates the possibility of structural modifications within the scope of the compounds of the invention being used to give spectral tuning, both for better biological light penetration (red-shifting), and so that both trans->cis and (especially) cis->trans photoisomerisations may be conducted more efficiently and more completely.

Photocharacterisation Results 3: Trans<->Cis Photoisomerisations are Fully Reversible Over Thousands of Cycles Under Biologically Relevant Conditions

A(λ_{strong}) was measured over time in the UV-Vis cuvette in non-degassed PBS left open to the atmosphere, containing 10% MeCN, at 37° C., while the monochromator was used to apply λ_{irrad} alternating between two values λ₁ and λ₂, chosen to induce net trans->cis and net cis->trans isomerisation respectively. Typical results are presented for compound I.1 (FIG. 3). The absorbance of I.1 (18 μM) was measured at λ_{strong}=378 nm, while the irradiating wavelength was held alternately at λ₁=388 nm (50 s; bulk trans->cis) then λ₂=508 nm (180 s, bulk cis->trans). Higher absorbance corresponds to a greater amount of trans isomer: an absorbance of 0.27 here corresponds to an approximately 5:1 ratio of trans:cis isomer in the sample, and an absorbance of 0.12 here corresponds to an approximately 1:2 ratio of trans:cis isomer in the sample. Note that this bulk trans->cis isomerisation at 388 nm is significantly faster than the bulk cis->trans isomerisation at 508 nm, although the monochromator equipment used delivers light at 508 nm with approximately 1.5 times the intensity of the light delivered at 388 nm. This is consistent with the expectation that photoisomerisation toward the respective photostationary states should be significantly more efficient at 388 nm than at 508 nm for this compound, since E(388)/E(508)=0.04. Typically, compounds of the invention could be bulk-photoisomerised hundreds of times over a timescale of hours (50 representative cycles are shown in FIG. 3 for I.1), such that the majority species in a sample was photocontrollably alternated between the trans-isomer and the cis-isomer.

However, the photoisomerisation behaviour of compound I.25, which features a reporter moiety (a rhodamine B fluorophore derivative) attached to the azoaryl moiety via a linker as explained in the Description, was qualitatively different in a very important fashion. As noted in Table 1 and shown in FIG. 4, the absorbance spectra of the trans and cis forms of I.25 are bimodal, with the region λ<450 nm dominated by the azoaryl moiety and the region λ>510 nm dominated by the rhodamine moiety. This particular rhodamine was chosen due to a literature report that its fluorescence emission occurs with high brightness (the product of the excitation wavelength absorption coefficient and the fluorescence quantum yield), and only above 525 nm (peak emission reported at 561 nm).^{[46]} Above 525 nm is a spectral region where the absorbance of only the as (but not the trans) isomers of prototypical azoaryl moieties of compounds of the invention (such as I.1) is non-negligible. Therefore it was considered possible that irradiation of a mixture of cis and trans isomers of I.25 in the spectral region dominated by the strongly-absorbing rhodamine moiety might result in favourable resonant energy transfer from the rhodamine moiety to the azoaryl moiety only when the azoaryl is in the as form, and that this energy transfer could result in isomerisation of the diazenyl bond, thus that such irradiation could rapidly decrease the subpopulation in the as form, perhaps even without substantial photoisomerisation of the subpopulation in the trans form. Firstly, irradiation at 554 nm of a sample of I.25 which had been kept in the dark resulted in visible fluorescence, but without the absorbance spectrum of the sample in the region 360-450 nm being changed. This is consistent with the hypothesis that irradiation at this wavelength does not substantially decrease the population of the trans form. As was observed previously with similar azoaryl moieties, such as for compound I.1, irradiation of this sample of I.25 at 384 nm induced a rapid decrease of the absorbance around 375 nm, consistent with the interpretation that irradiation at this wavelength effects bulk trans->cis photoisomerisation. Subsequent irradiation of this sample at 554 nm then resulted in the very rapid return of the absorbance spectrum of the sample entirely to its dark-adapted state (all-trans). This is consistent with the hypothesis that irradiation at this wavelength effects quantitative cis->trans photoisomerisation of I.25, presumably by highly selective resonant energy transfer to the cis isomer. For illustrative purposes, the A(λ_{strong}=375 nm) of I.25 was monitored while λ_{irrad} was alternated between λ₁=384 nm and λ₂=554 nm, and the results are also shown in FIG. 4.

The unusual rapidity and the quantitative nature of the cis->trans photoisomerisation of I.25 should especially be noted, and may be compared with the slower and non-quantitative nature of the cis->trans photoisomerisation of I.1 which was depicted in FIG. 3.

The cyclical photoisomerisations of all compounds tested proceeded without any detectable photobleaching, or decreases in photoswitching speed, or photoswitching efficiency or limiting isomeric percentage obtained, despite the fact that measurements were conducted in non-degassed aqueous solution under open air atmosphere at 37° C. This highlights the extreme robustness of the azoaryl compounds of the invention towards photochemical reaction or damage in a biologically relevant setting. This robustness is a key advantage of the compounds of the invention when compared to the prior art relying on stilbene photoisomerisation, which is a process known to give extensive and rapid degradation (principally to phenanthrenes) when biologically relevant conditions (eg. presence of dissolved oxygen) are used^{[11]}. These results thus demonstrate that photocontrolled trans<->cis isomerisation of the compounds of the invention is fully reversible, in a highly robust fashion which it is practical to implement under biologically relevant conditions.

### Photocharacterisation Results 4: Thermal Reversion Times

Thermal reversion times were measured as described above. Those for selected compounds are presented below (Table 2).

TABLE 2
reversion times for selected compounds,
measured in PBS at pH = 7.4 unless
indicated otherwise, containing 20%
MeCN to ensure solubility. “Too Fast”
indicates that no clear isosbestic point
could be established due to insufficient
measurable photoisomerisation, which was
attributed to very fast thermal reversion.

| Compound | τ (min) |
|---|---|
| I.1 | 6.2 |
| I.1, pH = 6 | 8.4 |
| I.2 | 0.75 |
| I.3 | 61 |
| I.4 | 119 |
| I.5 | 134 |
| I.6 | 83 |
| I.7 | 50 |
| I.8 | TOO FAST |
| I.9 | 116 |
| I.11 | 40 |
| I.12 | 28 |
| I.13 | 270 |
| I.14 | 3.3 |
| I.16 | TOO FAST |
| I.17 | TOO FAST |
| I.25 | 16.2 |
| I.26 | 8.4 |
| I.27 | 129 |
| I.28 | 248 |
| I.29 | 104 |

Table 2 shows that structural modifications within the scope of the compounds of the invention can greatly alter the timescale of spontaneous cis->trans reversion. Therefore different compounds of the invention may be appropriate for different types of biological experiments, especially when these are carried out over significantly different timescales (eg. seconds to minutes for experimental biology applications, or hours to days for biomedical applications), or if weak (ie. non-saturating) light intensities are to be used. Note that compounds I.8, I.16 and I.17 showed no measurable change in absorbance spectrum upon irradiation at 390 nm. This is interpreted as being due to very fast kinetics of spontaneous reversion which deplete the cis population before it can contribute to the absorbance; thus they are indicated to possess “too fast” a reversion time to be measured by this method. Proton transfer to the diazenyl bond from the hydroxyl or amino group in ortho to this diazenyl bond, is the assumed mechanism underlying these fast kinetics.

### Photocharacterisation Results 5: Fluorescence of I.25

Fluorescence imaging is commonly used to sensitively, conveniently and non-invasively determine the local concentration of fluorescent species in biological and medical settings. Considering I.25 as an example of a compound of the invention bearing a fluorescent reporter, it was considered desirable that its rhodamine moiety would allow fluorescence detection of I.25. Fluorescence excitation and emission spectra of I.25 were therefore acquired and are shown in FIG. 5.

The fluorescence emission spectra in the top panel of FIG. 5 show that I.25 can give a relatively broad fluorescence signal with an emission maximum at 590 nm when excited either at wavelengths which are also appropriate for effecting bulk trans->cis isomerisation (eg. 380 nm, dotted line), or else at wavelengths which are also appropriate for effecting near-quantitative cis->trans isomerisation (eg. 554 nm, solid line; note that the vertical scale is in arbitrary units not comparable between measurements).

The excitation spectrum of I.25 in the bottom panel of FIG. 5 shows the relative intensity of fluorescent emission at 590 nm, depending on the excitation wavelength used. While excitation at 570 nm gives the maximum emission intensity, many other wavelengths provide satisfactory fluorescent readout, eg. the spectral range between 350 nm and 440 nm, and that between 470 nm and 580 nm. It should also be noted that irradiation between 455-465 nm does not result in significant fluorescence output, which may be useful if azoaryl trans<->cis photoisomerisation is desired without risking fluorescent output.

Therefore, I.25 provides an example of a compound of the invention bearing a reporter chosen such that the fluorescence output of I.25 is well-defined, and can be produced by a broad range of excitation wavelengths covering much of the wavelength range commonly used for fluorescence imaging in biological and medical settings, and can be produced either by excitation wavelengths favouring the generation of the as isomer (eg. 384 nm) or favouring the generation of the trans isomer (eg. 554 nm), which factors should allow sophisticated biological applications eg. in fluorescent tracking, as well as benefiting from the advantage of resonant energy transfer allowing near-quantitative cis->trans photoisomerisation as described above.

### Part C: Biochemistry In Vitro

Turbidimetric tubulin polymerisation assays were performed as described in the literature^{[29]}, following the increase in absorbance at 340 nm, but with the addition of irradiation supplied by the monochromator setup described in Part B. Two wavelengths were chosen for each compound, λ_{E->Z} (chosen to effect bulk trans->cis isomerisation, typically 390 nm), and λ_{Z->E} (chosen to effect bulk cis->trans isomerisation, typically 505 nm). The raw absorbance A(t) could be more clearly discussed by first correcting for any change in azoaryl absorbance over time by subtracting the absorbance of a control run performed without tubulin, yielding the corrected absorbance A*(t), and then taking its derivative R=dA*(t)/dt. Experiments were performed under six sets of conditions to illustrate the light control of tubulin polymerisation inhibition effected by the compounds of the invention: (a) in the dark (typical result: large R at the start decreasing rapidly over time), or (b) with constant irradiation at λ_{E->Z} (typical result: small R throughout the experiment), or (c) with constant irradiation at λ_{Z->E} (typical result: moderate R at the start decreasing slowly over time), or (d) with irradiation at λ_{E->Z} for an initial period then darkness (typical result: initial period with small R, then R increases after a while), or (e) with irradiation at λ_{E->Z} for a given period (typically 30 min) then irradiation at λ_{Z->E} (typical result: 30 minutes with small R, then R increases quickly to a moderate value which decreases slowly over time), or (f) with irradiation at λ_{E->Z} for a given period (usually 30 min) then a short irradiation at λ_{Z->E} (usually 30 seconds) then darkness (typical result: 30 minutes with small R, then R increases quickly to a moderate-to-large value which decreases slowly over time); the last three conditions required correction of A(t) to A*(t) to reveal the changes in absorption which were due only to tubulin polymerisation. It would be costly to determine light-dependent in vitro tubulin polymerisation IC₅₀ values for each compound, eg. under protocols (a) and (b) (and/or (c)). This was not performed here since it was considered that such parameters have little predictive value in their intended complex biological settings, especially in light of the importance of biodistribution, tubulin-binding kinetics and lability (rather than thermodynamic binding strength) for tubulin disruption in biological settings.

Table 3 below illustrates typical results from experiments of type (a) (“DARK”) and (b) (“390 nm”) as described above, with compound (I.1) at concentrations well above the IC₅₀ for the toxic regime, compared to a PBS-only control CTRL (no I.1 present).

TABLE 3
A turbidimetric tubulin polymerization assay showing
the absorbance A(t) as defined above, comparing the
behaviour of a control run CTRL (no azoaryl added)
vs runs using compound I.1 at 50 and 25 μM,
with constant 390 nm illumination or else in the dark.

| | A(λ = 340 nm) | | | | |
|---|---|---|---|---|---|
| time | CTRL | I.1, 50 μM | | I.1, 25 μM | |
| (min) | DARK | DARK | 390 nm | DARK | 390 nm |
| 0.0 | 0.0000 | 0.0000 | 0.0000 | 0.0000 | 0.0000 |
| 0.5 | −0.0011 | 0.0010 | 0.0014 | 0.0001 | −0.0026 |
| 1.0 | −0.0015 | 0.0014 | 0.0020 | −0.0016 | −0.0022 |
| 1.5 | 0.0061 | 0.0034 | 0.0033 | 0.0018 | −0.0022 |
| 2.0 | 0.0220 | 0.0128 | 0.0027 | 0.0119 | −0.0010 |
| 2.5 | 0.0486 | 0.0305 | 0.0052 | 0.0274 | −0.0001 |
| 3.0 | 0.0754 | 0.0552 | 0.0066 | 0.0492 | 0.0004 |
| 3.5 | 0.1013 | 0.0734 | 0.0074 | 0.0689 | 0.0029 |
| 4.0 | 0.1195 | 0.0926 | 0.0100 | 0.0851 | 0.0027 |
| 4.5 | 0.1324 | 0.1046 | 0.0084 | 0.0984 | 0.0034 |
| 5.0 | 0.1398 | 0.1139 | 0.0122 | 0.1073 | 0.0100 |
| 5.5 | 0.1456 | 0.1209 | 0.0166 | 0.1133 | 0.0094 |
| 6.0 | 0.1474 | 0.1269 | 0.0163 | 0.1150 | 0.0132 |
| 6.5 | 0.1491 | 0.1284 | 0.0172 | 0.1212 | 0.0174 |
| 7.0 | 0.1537 | 0.1262 | 0.0213 | 0.1249 | 0.0171 |
| 7.5 | 0.1545 | 0.1379 | 0.0209 | 0.1228 | 0.0222 |
| 8.0 | 0.1553 | 0.1338 | 0.0221 | 0.1250 | 0.0241 |
| 8.5 | 0.1544 | 0.1359 | 0.0268 | 0.1259 | 0.0259 |
| 9.0 | 0.1559 | 0.1367 | 0.0268 | 0.1240 | 0.0288 |
| 9.5 | 0.1562 | 0.1374 | 0.0293 | 0.1276 | 0.0313 |
| 10.0 | 0.1552 | 0.1397 | 0.0320 | 0.1276 | 0.0341 |
| 10.5 | 0.1545 | 0.1370 | 0.0318 | 0.1255 | 0.0358 |
| 11.0 | 0.1576 | 0.1405 | 0.0327 | 0.1290 | 0.0402 |
| 11.5 | 0.1556 | 0.1417 | 0.0333 | 0.1299 | 0.0413 |
| 12.0 | 0.1584 | 0.1402 | 0.0359 | 0.1333 | 0.0443 |
| 12.5 | 0.1567 | 0.1444 | 0.0391 | 0.1330 | 0.0457 |
| 13.0 | 0.1588 | 0.1440 | 0.0385 | 0.1317 | 0.0473 |
| 13.5 | 0.1569 | 0.1483 | 0.0381 | 0.1311 | 0.0519 |
| 14.0 | 0.1577 | 0.1453 | 0.0420 | 0.1355 | 0.0539 |
| 14.5 | 0.1567 | 0.1478 | 0.0436 | 0.1345 | 0.0531 |
| 15.0 | 0.1592 | 0.1515 | 0.0448 | 0.1403 | 0.0576 |

Table 3 indicates that tubulin polymerisation is very strongly inhibited by (I.1) in a dose-dependent fashion when it is exposed to 390 nm irradiation, but is not inhibited at these concentrations in the dark. This can be understood as a strong tubulin polymerisation inhibition effected only by (I.1)-cis, since if darkness is maintained, (I.1)-trans is the isomeric form present in the sample, and tubulin polymerisation is seen here to proceed identically to the control case.

These results indicate that the compounds of the invention can act in vitro as inhibitors of tubulin polymerisation whose inhibitory activity may be controlled (activated or deactivated) by the choice of illumination conditions over time.

### Part D: Cell Biology

### From Photopharmacology Principles to Biology Assay Design:

For clarity of discussion, [Z] is defined as the instantaneous local concentration of the cis-azoaryl isomer; [Z]* is defined as the time-average [Z] experienced during a significant phase of an experiment (eg. the first phase of a two-phase experiment; see below); and tₚₐᵤₛₑ t is defined as the interval between light pulses in a pulsed experiment (if the experiment is a dual-wavelength experiment, each pulse is defined as containing both λ_{ACT} and λ_{DEACT}). Recall that the target is defined as the spatiotemporal region where it is desired that the biological effects of the azoaryl compound be most strongly applied, while it is considered beneficial to avoid generating biological effects in off-target zones, whether far from or near to the target.

Two strong light-dependent steady-state biomedically relevant protocols are evident: (1) a toxic regime designed to maximise the pharmacological toxicity of the azoaryl compound, by applying activating irradiation at wavelength λ_{ACT} so as to generate a significant [Z]* in the target; continuous irradiation could be used, or else pulsed irradiation if tₚₐᵤₛₑ were significantly shorter than τ; an example pulsed toxic regime for compound I.1 (τ estimated as 6 minutes) could thus be “390 nm applied in pulses of 200 ms with tₚₐᵤₛₑ=30 s”; and (2) a strong rescue regime, designed to deliver a rigorous test of the degree of photocontrol which can be exerted over the azoaryl compound's toxicity, by applying λ_{ACT} as for the toxic regime, but competitively applying deactivating irradiation at wavelength λ_{DEACT} in order to reduce [Z]* relative to the value experienced in the toxic regime; like the toxic regime, this regime could be pulsed or continuous; an example pulsed strong rescue regime for compound I.1 could thus be “390 nm applied for 200 ms then 505 nm applied for 600 ms, with tₚₐᵤₛₑ=30 s”.

One likely design for localised therapeutic applications of the compounds of the invention is by spatially separated application of a toxic regime on a target synchronously with the application of a deactivating regime (featuring only the λ_{DEACT} component of an optimised strong rescue regime) in a thin protection zone surrounding this target (in order to reduce the exposure of the rest of the organism or sample to any cis-azoaryl isomer escaping the target). Especially with strongly nonlinear dose-response relationships, as the present compounds feature, this may maximise the biological effects in a target while keeping the off-target [Z]* below the minimal response concentration, thereby avoiding side effects. In the context of biomedical applications therefore, the toxic regime thus gives an estimate of the maximum strength of the biological effects that can be exerted in a target zone by a given concentration of the azoaryl compound; and assuming that a deactivating regime can be applied in those off-target zones which are the very closest neighbours to this target zone, then the strong rescue regime estimates the maximum strength of the (undesirable) biological effects that could be experienced in the very nearest off-target zones, eg. due to the diffusion of cis isomer out of the target zone or due to a degree of scattering of λ_{ACT}; weaker biological effects are to be anticipated in off-target zones still further from the target.

Weak light-dependent protocols are defined as those where spontaneous reversion plays a significant role in reducing [Z]*, and these may also be very important in medical and especially fundamental research applications. Examples include (3) a dark rescue regime, where a toxic regime would be applied for the first phase of an experiment, then all light switched off and darkness maintained throughout a second phase of the experiment thus allowing [Z] to reach zero; and (4) a weak pulsed rescue regime similar to the strong pulsed rescue regime but where tₚₐᵤₛₑ t is instead significantly longer than τ, such that the component of λ_{DEACT} in each pulse primes the sample to decay more rapidly to [Z]˜0 than would be possible by spontaneous reversion alone. Note that a weak pulsed rescue regime will always display lesser biological effects than the corresponding strong rescue regime which has a shorter tₚₐᵤₛₑ. Note also that dynamic protocols exploiting the spatiotemporal control of appropriate irradiation could find even more sophisticated applications than these steady-state protocols.

In any chosen regime, light could be applied either continuously (allowing very low intensities to be used), or in pulses (allowing for fixed source intensity and probably permitting higher-performance implementation of rescue protocols). Example experiments showed that pulsed protocols were well-adapted to illustrating the relationship between observed photopharmacological effects and the underlying qualitative trends of PSS(λ), E(λ) and τ described previously, so pulsed protocols were used throughout biological studies. Results from the strong light-dependent steady-state protocols are given here, as these provide a more demanding proof of the principle of fully reversibly light-controllable biological effects than do the weak protocols described above. The predictable and successful outcome of experiments under these protocols serves as a generalised indication that weak protocols can also be applied with at least as much, if not more, success. Lastly, note that λ_{E->Z} and λ_{Z->E} as defined above also give initial estimates for “biologically good” values of λ_{ACT} and λ_{DEACT} by balancing the need to restrict the light flux applied, while still favouring one or the other isomer's formation as much as possible. The values of λ_{ACT} and λ_{DEACT} used in optimised experiments could be refined empirically from these initial guesses, however this was typically not necessary.

### Use of Photoswitchable Compounds in Cell Culture:

Irradiated cell culture was performed using a self-built computer-controlled system of arrays of LEDs, where each array irradiated a standard 24-well or 96-well cell culture plate, and these were contained in separate light-proof gas-permeable boxes in a cell culture incubator; the system is illustrated schematically in FIG. 6. Either one or two arrays were conveniently used per wellplate (typically, an array at an activating wavelength illuminating from the bottom, with an optional second array at a deactivating wavelength illuminating from the top down on a different timing sequence if desired), thus enabling pulsed or continuous implementation of eg. toxic regime, strong rescue regime, weak rescue regime, or dark rescue regime protocols, in a straightforward manner.

### Results 1: Photoactuated Toxicity of a Range of Compounds:

Seven of the compounds according to the invention were selected for biological tests. Their cytotoxicity was assessed using crystal violet staining as adapted from standard procedure^{[31]}. Briefly, HeLa cells were seeded on 96-well plates, treated with the given concentrations of the selected compound, and exposed or not to the pulse protocol of illumination with light at 390 nm (pulses of 75 ms every 15 s). After 40 h cells were stained with crystal violet solution (0.5% crystal violet in 20% methanol) for 10 min. Unbound crystal violet was removed by rinsing with distilled water and cells were air-dried. Crystal violet was subsequently eluted from cells with 0.1 M sodium citrate in 50% ethanol. The absorbance of crystal violet is proportional to the cell number and was determined at 590 nm with a FLUOstar Omega microplate reader (BMG Labtech).

Each compound was tested at 6 doses: 100 nM, 500 nM, 1 μM, 2 μM, 5 μM and 10 μM. In order to ensure their solubility in the cell culture media, DMSO was used as a co-solvent, and to provide comparability between all samples the volume of DMSO was adjusted to obtain its final concentration as 1% in the cell culture media for all the compounds at all concentrations tested.

Results presented in Table 4 below are expressed as a fold growth relative to the control growth of the cells treated only with a co-solvent (1% DMSO), and are represented as a mean value from triplicates coming from a representative experiment.

TABLE 4
Cytotoxicity assessed by crystal violet staining, followed 40 h
of treatment with indicated concentrations of the panel of
indicated compounds in the dark or upon application of a pulsed
toxic regime protocol (390 nm for 75 ms every 15 s).

| | | | Dark | | 390 nm | |
|---|---|---|---|---|---|---|
| | | | mean | | mean | |
| | I.1 | | fold | | fold | |
| No. | concentration | | growth | StDev | growth | StDev |
| 1 | 0 | nM | 1.00 | 0.05 | 1.00 | 0.006 |
| 2 | 100 | nM | 0.95 | 0.04 | 0.95 | 0.04 |
| 3 | 500 | nM | 0.89 | 0.008 | 0.85 | 0.03 |
| 4 | 1 | μM | 0.87 | 0.03 | 0.43 | 0.01 |
| 5 | 2 | μM | 0.89 | 0.02 | 0.35 | 0.004 |
| 6 | 5 | μM | 0.84 | 0.009 | 0.27 | 0.04 |
| 7 | 10 | μM | 0.75 | 0.02 | 0.35 | 0.04 |
| | | | Dark | | 390 nm | |
| | | | mean | | mean | |
| | I.2 | | fold | | fold | |
| No. | concentration | | growth | StDev | growth | StDev |
| 1 | 0 | nM | 1.00 | 0.03 | 1.00 | 0.04 |
| 2 | 100 | nM | 0.97 | 0.04 | 1.10 | 0.16 |
| 3 | 500 | nM | 0.90 | 0.02 | 0.83 | 0.07 |
| 4 | 1 | μM | 0.89 | 0.04 | 0.50 | 0.07 |
| 5 | 2 | μM | 0.81 | 0.03 | 0.47 | 0.05 |
| 6 | 5 | μM | 0.86 | 0.008 | 0.32 | 0.08 |
| 7 | 10 | μM | 0.70 | 0.05 | 0.34 | 0.06 |
| | | | Dark | | 390 nm | |
| | | | mean | | mean | |
| | I.3 | | fold | | fold | |
| No. | concentration | | growth | StDev | growth | StDev |
| 1 | 0 | nM | 1.00 | 0.08 | 1.00 | 0.08 |
| 2 | 100 | nM | 1.03 | 0.04 | 0.93 | 0.08 |
| 3 | 500 | nM | 1.00 | 0.01 | 0.88 | 0.06 |
| 4 | 1 | μM | 0.95 | 0.03 | 0.82 | 0.06 |
| 5 | 2 | μM | 0.90 | 0.02 | 0.62 | 0.06 |
| 6 | 5 | μM | 0.96 | 0.02 | 0.39 | 0.02 |
| 7 | 10 | μM | 0.99 | 0.04 | 0.47 | 0.07 |
| | | | Dark | | 390 nm | |
| | | | mean | | mean | |
| | I.4 | | fold | | fold | |
| No. | concentration | | growth | StDev | growth | StDev |
| 1 | 0 | nM | 1.00 | 0.09 | 1.00 | 0.08 |
| 2 | 100 | nM | 0.93 | 0.04 | 0.98 | 0.11 |
| 3 | 500 | nM | 0.93 | 0.08 | 0.78 | 0.02 |
| 4 | 1 | μM | 0.89 | 0.07 | 0.53 | 0.08 |
| 5 | 2 | μM | 0.94 | 0.07 | 0.48 | 0.04 |
| 6 | 5 | μM | 0.94 | 0.07 | 0.44 | 0.06 |
| 7 | 10 | μM | 0.78 | 0.07 | 0.36 | 0.09 |
| | | | Dark | | 390 nm | |
| | | | mean | | mean | |
| | I.6 | | fold | | fold | |
| No. | concentration | | growth | StDev | growth | StDev |
| 1 | 0 | nM | 1.00 | 0.03 | 1.00 | 0.02 |
| 2 | 100 | nM | 1.00 | 0.03 | 0.99 | 0.02 |
| 3 | 500 | nM | 1.07 | 0.02 | 1.09 | 0.04 |
| 4 | 1 | μM | 0.96 | 0.01 | 0.79 | 0.11 |
| 5 | 2 | μM | 0.96 | 0.01 | 0.47 | 0.07 |
| 6 | 5 | μM | 0.93 | 0.05 | 0.68 | 0.06 |
| 7 | 10 | μM | 0.88 | 0.14 | 0.37 | 0.12 |
| | | | Dark | | 390 nm | |
| | | | mean | | mean | |
| | I.20 | | fold | | fold | |
| No. | concentration | | growth | StDev | growth | StDev |
| 1 | 0 | nM | 1.00 | 0.04 | 1.00 | 0.01 |
| 2 | 100 | nM | 0.99 | 0.06 | 1.09 | 0.11 |
| 3 | 500 | nM | 1.11 | 0.05 | 1.10 | 0.19 |
| 4 | 1 | μM | 1.12 | 0.12 | 1.00 | 0.14 |
| 5 | 2 | μM | 1.10 | 0.04 | 0.96 | 0.04 |
| 6 | 5 | μM | 1.03 | 0.04 | 0.68 | 0.02 |
| 7 | 10 | μM | 1.01 | 0.03 | 0.51 | 0.06 |
| | | | Dark | | 390 nm | |
| | | | mean | | mean | |
| | I.10 | | fold | | fold | |
| No. | concentration | | growth | StDev | growth | StDev |
| 1 | 0 | nM | 1.00 | 0.008 | 1.000 | 0.05 |
| 2 | 100 | nM | 0.95 | 0.08 | 0.986 | 0.08 |
| 3 | 500 | nM | 0.99 | 0.01 | 0.945 | 0.03 |
| 4 | 1 | μM | 0.92 | 0.07 | 0.387 | 0.02 |
| 5 | 2 | μM | 0.90 | 0.009 | 0.357 | 0.03 |
| 6 | 5 | μM | 0.99 | 0.18 | 0.217 | 0.03 |
| 7 | 10 | μM | 0.78 | 0.08 | 0.293 | 0.009 |

For all the compounds tested, strong dose-dependent toxicity was observed with activating irradiation (390 nm regimen), while in the dark no such significant cytotoxic effect was observed. The results of this experiment therefore support the conclusion that the compounds of the invention act as light-inducible cytotoxins. Based on the strong performance of compound I.1 and the similarity of its behavior to the other compounds tested, further tests in detail were pursued focusing on compound I.1 exclusively.

### Results 2: Photocontrolled Cytoxicity of I.1

The cytotoxic properties of I.1 were subsequently confirmed with another method, using the quantification of mitochondrial dehydrogenase activity of cells as determined by the level of 3-(4.5-dimethylthiazol-2-yl)-2.5-diphenyl tetrazolium bromide (MTT) reduction to its purple formazan, according to standard protocol^{[30]}. Briefly, HeLa cells were seeded on 96-well plates and treated with compound I.1 at concentrations ranging from 10 nM-2 μM in PBS containing 0.5% MeCN to ensure the compound's solubility. Cells were kept in the dark, or exposed to a pulsed toxic regime of 390 nm only, or exposed to a strong rescue protocol with pulses of 390 nm then 505 nm light. Pulses of light were applied every 30 s; 390 nm light pulses lasted 150 ms every 30 s, while the 505 nm light pulses were applied for 500 ms; in the strong rescue regime, the 505 nm pulse was synchronised so that it began immediately after the 390 nm pulse ended.

Cellular viability was measured 48 h later, namely, cells were incubated with MTT for three hours and after dissolution of the formazan crystals with DMSO, absorbance at 550 nm was measured using a FLUOstar Omega microplate reader. Results are shown in Table 5 below and are expressed as a fold growth relative to the control growth of the cells treated only with a co-solvent (0.5% MeCN) and represent mean and standard deviation of quadruplicates of the optical density values, which are proportional to the cell number.

TABLE 5
Values of formazan absorbance measured by MTT assay
were used to derive fold values correlated to cell count number,
which were used to quantify the cytotoxicity of compound I.1.
Light pulses were applied every 30 s; 390 nm light pulses
lasted 150 ms, 505 nm light pulses lasted 500 ms.

| | | | | | | | rescue: | |
|---|---|---|---|---|---|---|---|---|
| | | | | | | | 390 nm then | |
| | | | Dark | | 390 nm | | 505 nm | |
| | | | mean | | mean | | mean | |
| | | | fold | | fold | | fold | |
| No. | compound | | growth | StDev | growth | StDev | growth | StDev |
| 1 | I.1 0 | nM | 1.00 | 0.08 | 1.00 | 0.05 | — | — |
| 2 | I.1 10 | nM | 0.93 | 0.08 | 0.95 | 0.03 | — | — |
| 3 | I.1 20 | nM | 1.02 | 0.04 | 0.96 | 0.03 | — | — |
| 4 | I.1 50 | nM | 1.01 | 0.03 | 0.96 | 0.04 | — | — |
| 5 | I.1 100 | nM | 1.01 | 0.05 | 0.93 | 0.02 | — | — |
| 6 | I.1 400 | nM | 1.00 | 0.05 | 0.28 | 0.02 | 0.46 | 0.04 |
| 7 | I.1 600 | nM | 1.05 | 0.06 | 0.21 | 0.01 | — | — |
| 8 | I.1 800 | nM | 0.98 | 0.07 | 0.17 | 0.02 | — | — |
| 9 | I.1 1 | μM | 0.93 | 0.02 | 0.18 | 0.02 | — | — |
| 10 | I.1 2 | μM | 0.98 | 0.03 | 0.21 | 0.01 | — | — |
| 11 | CA4P 50 | nM | 0.29 | 0.05 | 0.38 | 0.10 | — | — |

In the dark no toxicity of compound I.1 was observed within the concentration range tested, while upon exposure to the light, compound I.1 showed an induction of severe cytotoxicity at a concentration ≧400 nM and the cytotoxic effect was comparable to that of the 50 nM CA4P positive control (Table 5). In the strong rescue protocol, tested here only with compound I.1 at 400 nM, approximately 5700 pulses were applied over the 48 hours of the experiment, each time cycling between bulk trans->cis then bulk cis->trans photoisomerisation. It should be noted that the rescue protocol cells were under irradiation for significantly longer than the toxic protocol cells due to the double irradiation, however this rescue protocol reduced the cytotoxic effect of compound I.1 compared to the 390 nm-only protocol, which is strong evidence of the possibility of fully reversible light-control of the anti-proliferative properties of compound I.1.

### Results 3: Cell Cycle Arrest

The effect of compound I.1 on cell cycle progression was assessed by flow cytometry. Briefly, following the application of compound I.1 and exposure to the indicated light regime, cells were harvested on ice and incubated in a hypotonic buffer [0.1% sodium citrate, 0.1% Triton X-100 and 50 μg/mL propidium iodide (PI)] for 30 min at 4° C. Following the PI staining cells were analysed by flow cytometry using FACSCalibur flow cytometer (Becton Dickinson, Heidelberg, Germany) and Cell Quest Pro Software (Becton Dickinson). Subsequently the cell cycle analysis was performed using the FlowJo software (Tree Star Inc., Ashland, Oreg., USA).

As is typical for tubulin-binding agents, compound I.1 induced cell cycle arrest in G₂/M phase, but for compound I.1 this was dependent on the light regimen. Table 6 below shows the repartition of cells between different phases of the cell cycle. This shows dose-dependent arrest in G₂/M phase in MDA-MB-231 cells treated with compound I.1 and exposed to a 390 nm toxic regime. This effect is not cell type specific: it was observed in all the tested cell types MDA-MB-231, HEK-293, HeLa and HepG2 (Tables 7-8); a significantly higher response threshold was only observed for the HepG2 cells. Therefore the invention shows a light-activated mode of action, and one which is generalizable across cell types (as appropriate to their generalisable mechanism of cytotoxicity).

Controls supported these experimental conclusions as to the light-dependency of the cytotoxicity of compound I.1. Analysis of control cells treated with CA4P showed the same level of cell cycle arrest regardless of illumination conditions; notably though, when compound I.1 was not subjected to light, no cell cycle arrest was evident within the whole concentration range tested (Table 7 and Table 8 below).

Again, compound I.1 was shown to be able to induce toxicity in a more sophisticated light-dependent manner when different wavelengths were considered. Table 9 below presents results again illustrating that the biological effects induced under the toxic regime can be reduced by applying a strong rescue regime. This strong rescue protocol succeeds in reducing the toxicity to levels approximately the same as are seen when only the “deactivating” pulse component is applied, although applying a total dose of light approximately four times higher than in the toxic regime (either in terms of illumination time and/or applied energy), which supports the idea that the cis-form of compound I.1 was the main determinant of toxicity. Again, the rescue protocol thus isomerised the compounds of the invention, to a proportion clearly significant for determining toxicity, back and forth between cis and trans forms more than 5000 times over the experiment, thereby supporting the claim of full and reversible light control of the toxicity of the compounds of the invention, demonstrable in a robust and practical setting.

Result values in Tables 6-9 below are presented as mean+/−standard deviation calculated for triplicates from one representative experiment out of three independent trials.

TABLE 6
Repartition of cells between the phases of the cell cycle in
MDA-MB-231 cells treated with compound I.1 and
exposed for 48 h to 390 nm regimen (150 ms pulses every 30 s).

| | Concen- | | 390 nm | | | | | |
|---|---|---|---|---|---|---|---|---|
| | tration | | % G1 | | % S | | % G2 | |
| No | of I.1 | | mean | StDev | mean | StDev | mean | StDev |
| 1 | 0 | | 77.4 | 7.1 | 16.9 | 1.5 | 2.7 | 0.5 |
| 2 | 10 | nM | 74.6 | 6.0 | 17.1 | 0.8 | 1.9 | 0.3 |
| 3 | 20 | nM | 79.5 | 8.7 | 18.0 | 1.9 | 2.5 | 0.6 |
| 4 | 50 | nM | 77.1 | 4.8 | 18.0 | 1.9 | 1.8 | 0.2 |
| 5 | 100 | nM | 76.6 | 4.8 | 18.5 | 1.9 | 2.0 | 0.3 |
| 6 | 400 | nM | 53.1 | 3.6 | 27.6 | 0.7 | 15.1 | 1.4 |
| 7 | 600 | nM | 18.0 | 1.6 | 31.0 | 0.4 | 42.6 | 0.8 |
| 8 | 800 | nM | 9.5 | 4.0 | 27.5 | 2.1 | 54.2 | 10.9 |
| 9 | 1 | μM | 5.5 | 0.7 | 27.6 | 3.4 | 52.9 | 3.9 |
| 10 | 2 | μM | 4.8 | 0.2 | 26.4 | 4.0 | 60.1 | 1.3 |
| 11 | 5 | μM | 5.0 | 0.7 | 23.9 | 2.1 | 70.2 | 9.1 |

TABLE 7
G2/M phase arrest in the panel of cell lines:
HEK-293, HeLa and MDA-MB-231. Cells were exposed
to compound I.1 at indicated concentrations and exposed to a
390 nm regimen (1 s pulses every 15 min), or not (“dark”). The
cell cycle analysis was performed 30 h post-treatment.

| | | | | HEK | | HeLa | | MDA | |
|---|---|---|---|---|---|---|---|---|---|
| | | | | mean % | | mean % | | mean % | |
| | light | | | G2/M | | G2/M | | G2/M | |
| No. | type | Compound | | phase | StDev | phase | StDev | phase | StDev |
| 1 | dark | — | | 19.8 | 0.4 | 22.7 | 0.4 | 14.1 | 0.9 |
| 2 | dark | I.1 200 | nM | 20.2 | 0.4 | 23.5 | 0.9 | 14.3 | 0.9 |
| 3 | dark | I.1 600 | nM | 21.2 | 0.3 | 24.8 | 0.3 | 12.8 | 1.5 |
| 4 | dark | I.1 1.5 | μM | 21.1 | 0.1 | 27.1 | 0.5 | 13.1 | 0.6 |
| 5 | dark | I.1 5 | μM | 24.6 | 0.07 | 28.4 | 0.8 | 13.8 | 1.4 |
| 6 | dark | CA4P 15 | nM | 72.0 | 0.5 | 69.0 | 1.2 | 51.3 | 2.3 |
| 7 | 390 nm | — | | 18.5 | 0.5 | 24.4 | 0.4 | 10.5 | 3.3 |
| 8 | 390 nm | I.1 200 | nM | 19.0 | 0.4 | 26.2 | 0.6 | 8.2 | 2.0 |
| 9 | 390 nm | I.1 600 | nM | 19.4 | 1.6 | 33.7 | 1.8 | 16.3 | 0.07 |
| 10 | 390 nm | I.1 1.5 | μM | 65.3 | 3.0 | 59.9 | 2.1 | 40.6 | 2.6 |
| 11 | 390 nm | I.1 5 | μM | 71.5 | 2.6 | 68.7 | 0.9 | 49.4 | 1.9 |
| 12 | 390 nm | CA4P 15 | nM | 70.1 | 5.5 | 69.5 | 1.1 | 50.9 | 1.6 |

TABLE 8
Cell cycle analysis of HepG2 cells following 48 h
treatment with compound I.1 and exposure to a
390 nm light regimen (250 ms pulses every 15 s)
or not (“dark”).

| | | | | | HepG2 | |
|---|---|---|---|---|---|---|
| | | | | | mean % | |
| | lighting | | | | G2/M | |
| No | type | | Compound | | phase | StDev |
| 1 | dark | | — | | 9.9 | 0.5 |
| 2 | dark | | 1.1 100 | nM | 10.0 | 1.0 |
| 3 | dark | | 1.1 600 | nM | 8.6 | 0.9 |
| 4 | dark | | 1.1 1 | μM | 10.1 | 0.5 |
| 5 | dark | | 1.1 3 | μM | 11.2 | 2.1 |
| 6 | dark | | 1.1 10 | μM | 11.7 | 1.3 |
| 7 | 390 | nm | — | | 8.7 | 1.2 |
| 8 | 390 | nm | I.1 100 | nM | 7.8 | 0.6 |
| 9 | 390 | nm | I.1 600 | nM | 9.4 | 0.7 |
| 10 | 390 | nm | I.1 1 | μM | 10.7 | 1.5 |
| 11 | 390 | nm | I.1 3 | μM | 9.3 | 1.5 |
| 12 | 390 | nm | I.1 10 | μM | 42.3 | 2.8 |

TABLE 9
Cell cycle analysis of MDA-MB-231 cells treated with compound I.1 and exposed
for 48 h to a 390 nm regimen (a 150 ms pulse at 390 nm every 30 s), or a 515 nm
regimen (a 500 ms pulse at 515 nm every 30 s), or a rescue regimen (a 150 ms pulse at
390 nm then immediately a 500 ms pulse at 515 nm, one such pulse pair every 30 s).

| | | | | | | | 390 nm + 515 nm | |
|---|---|---|---|---|---|---|---|---|
| | | | 390 nm | | 515 nm | | (“rescue”) | |
| | | | mean % | | mean % | | mean % | |
| | | | G2/M | | G2/M | | G2/M | |
| No | Compound | | phase | StDev | phase | StDev | phase | StDev |
| 1 | — | | 2.7 | 0.5 | 1.6 | 1.5 | 1.2 | 0.6 |
| 2 | I.1 10 | nM | 1.9 | 0.3 | 1.1 | 1.1 | 0.8 | 0.5 |
| 3 | I.1 100 | nM | 2.0 | 0.3 | 1.2 | 1.2 | 0.9 | 0.5 |
| 4 | I.1 400 | nM | 15.1 | 1.4 | 8.2 | 9.7 | 6.4 | 4.5 |
| 5 | I.1 600 | nM | 42.6 | 0.8 | 21.7 | 29.5 | 17.3 | 14.8 |
| 6 | I.1 800 | nM | 54.2 | 10.9 | 32.5 | 30.7 | 24.7 | 12.0 |
| 7 | I.1 1 | μM | 52.9 | 3.9 | 28.4 | 34.7 | 22.3 | 16.3 |

### Results 4: Light-Dependent Cell Proliferation Studied in Depth:

MTT assays on HEK-293T cells were performed with the MTT-assay procedure described in Results 2 above, but examining the light-dependency of the antiproliferative effect of compound I.1 in more depth. Cells were incubated in the dark, or with pulses of light at single wavelengths ranging from 525 nm to 390 nm, or under strong rescue regimes. The results are presented alongside the appropriate E(λ) values from the in vitro modelling above (Table 10 below).

TABLE 10
HEK 293T cells were treated for 72 h with compound I.1 while
being exposed to different irradiation patterns, each of which was applied
every 2 min (pulse durations and wavelengths are as indicated in column headings).

| | | | | | | | | | 390 nm, | |
|---|---|---|---|---|---|---|---|---|---|---|
| | | | | | | | | | 0.35s | 410 nm, |
| | | | | | | | | | then | 0.35 s |
| | | | 525 | 505 | 490 | 475 | 410 | 390 | 490 nm, | then 505 |
| | | | nm, | nm, | nm, | nm, | nm, | nm, | 3 s | nm, 3 s |
| HEK | | Dark | 3 s | 3 s | 3 s | 3 s | 0.35 s | 0.35 s | (rescue) | (rescue) |
| PBS only | | 0.99 | 1.18 | 1.12 | 1.21 | 1.03 | 1.06 | 1.18 | 1.27 | 1.16 |
| Cosolvent | | 1.00 | 1.00 | 1.00 | 1.00 | 1.00 | 1.00 | 1.00 | 1.00 | 1.00 |
| CA4P 5 | nM | 0.82 | 0.86 | 1.07 | 1.43 | 1.12 | 1.19 | 1.29 | 1.28 | 1.20 |
| CA4P 15 | nM | 0.79 | 0.81 | 0.75 | 0.97 | 0.70 | 0.80 | 0.88 | 0.82 | 0.81 |
| I.1 100 | nM | 1.29 | 1.12 | 1.22 | 1.10 | 1.11 | 1.19 | 1.31 | 1.26 | 1.29 |
| I.1 400 | nM | 0.87 | 0.82 | 0.87 | 0.69 | 0.55 | 0.70 | 0.67 | 0.69 | 0.86 |
| I.1 800 | nM | 0.95 | 1.00 | 0.92 | 0.43 | 0.37 | 0.51 | 0.38 | 0.33 | 0.67 |
| I.1 1.5 | μM | 0.71 | 0.66 | 0.42 | 0.19 | 0.19 | 0.25 | 0.22 | 0.18 | 0.25 |
| I.1 3 | μM | 0.66 | 0.64 | 0.27 | 0.20 | 0.15 | 0.18 | 0.17 | 0.14 | 0.22 |
| I.1 6 | μM | 0.80 | 0.39 | 0.27 | 0.34 | 0.21 | 0.23 | 0.29 | 0.18 | 0.30 |
| I.1 15 | μM | 0.87 | 0.29 | 0.20 | 0.31 | 0.17 | 0.23 | 0.28 | 0.21 | 0.24 |

| | | | | | | | |
|---|---|---|---|---|---|---|---|
| E(λ) | — | 0.02 | 0.04 | 0.08 | 0.14 | 0.59 | 1.00 |

It was observed that under the given experimental conditions, to achieve high cytotoxicity, either a high-efficiency wavelength could be applied in short pulses (eg. 390 nm or 410 nm), or else relatively long pulses of less efficient wavelengths could be applied (compare results for 3 s pulses of 475 nm with those for 0.35 s pulses of 410 nm), or else high doses of the compound of the invention could be applied even with a low-efficiency wavelength (results for 525 nm irradiation with 6 μM of compound I.1 are similar to those for 390 nm with 800 nM). It was also observed that the rescue protocol using 490 nm was not successful in reducing toxicity, while the rescue using 505 nm was successful (each considered relative to the toxicity given by their activating wavelength components, 390 nm and 410 nm respectively; see eg. the effects at 400-800 nM where the toxicity difference is clearest). The difference in rescue success can be understood by reference to the relative difference in the calculated values φ(490)=0.30 and φ(505)=0.23 (see Photocharacterisation Results). 505 nm light is thereby understood as producing a more complete overall isomerisation of active cis isomer towards the inactive trans isomer than 490 nm, despite its comparatively lower overall efficiency of producing photoisomerisation at all. These data are therefore consistent with the hypothesis that a certain threshold amount of cis isomer, formed by overall light-induced trans->cis isomerism, is needed to achieve a cytotoxic effect, and that this threshold may be reached by modulating the combination of (a) the total dose of the compound applied, (b) the wavelength(s) applied (with regards to their E(λ) and PSS(λ)), and (c) the time-average duration of each applied wavelength(s). This highlights the principle of rationally-understood light control of the compounds of the invention, as well as their reversible light control. It also shows that lighting and dose conditions to be applied can be chosen and rationally tuned to respond to experimental constraints while achieving the desired biochemical outcome, and that such tuning can be understood to depend on the key isomerisation parameters which can be influenced by chemical design^{[38,48]} and then measured experimentally as performed above in vitro.

### Results 5: Membrane Permeability

Membrane integrity was assessed as a marker of cellular viability, via examining the uptake of propidium iodide (PI) in nonpermeabilized cells according to a standard protocol. Namely, cells were harvested and incubated with 5 μg/mL PI in PBS containing 2% FCS (foetal calf serum), and immediately analysed by flow cytometry using a FACSCalibur flow cytometer.

The effect of compound I.1 was analysed in three cell lines: HeLa and MDA-MB-231 (Table 11 below) and Jurkat cells (Table 12 below). Treatment with compound I.1 combined with exposure to a 390 nm regimen led to increased membrane permeability in all cell lines tested. Similarly to the other experiments presented in this section, no dose-dependent toxicity of compound I.1 was observed in the dark. Therefore the cytotoxic effect of compound I.1 was light dependent. This is in notable contrast to the prior art of “always-active” compounds, as represented here by CA4P, which in both lighting conditions showed the same level of toxicity.

Data presented in Table 11 and Table 12 below are expressed as means and standard deviations of one representative experiment out of three independent trials performed in triplicate.

TABLE 11
The effects of compound I.1 on cell membrane permeability are presented.
HeLa and MDA-MB-231 cells were treated for 70 h with compound I.1
while being exposed to irradiation at 390 nm (1 s every 15 min), or not (“dark”).
The percentage of PI positive cells in the total amount of cells is shown.

| | | | | HeLa | | MDA-MB-231 | |
|---|---|---|---|---|---|---|---|
| | light | | | mean % | | mean % | |
| No | conditions | Compound | | cells PI+ | StDev | cells PI+ | StDev |
| 1 | dark | — | | 4.2 | 0.1 | 5.1 | 0.7 |
| 2 | dark | I.1 200 | nM | 4.5 | 0.02 | 5.0 | 0.9 |
| 3 | dark | I.1 600 | nM | 4.3 | 0.3 | 4.6 | 0.2 |
| 4 | dark | I.1 1.5 | μM | 4.2 | 0.5 | 4.3 | 0.4 |
| 5 | dark | I.1 5 | μM | 5.1 | 0.1 | 5.0 | 0.5 |
| 6 | dark | CA4P 15 | nM | 62.5 | 0.8 | 45.1 | 0.9 |
| 7 | 390 nm | — | | 4.1 | 0.6 | 5.0 | 1.6 |
| 8 | 390 nm | I.1 200 | nM | 4.5 | 0.1 | 4.4 | 0.8 |
| 9 | 390 nm | I.1 600 | nM | 8.2 | 0.1 | 8.2 | 0.5 |
| 10 | 390 nm | I.1 1.5 | μM | 46.1 | 0.7 | 24.1 | 1.3 |
| 11 | 390 nm | I.1 5 | μM | 73.0 | 0.07 | 47.2 | 1.7 |
| 12 | 390 nm | CA4P 15 | nM | 63.9 | 1.3 | 43.2 | 1.8 |

TABLE 12
The effects of compound I.1 on cell membrane permeability
in Jurkat cells treated for 48 h with compound I.1 and
exposed to irradiation at 390 nm (350 ms every 5 min)
or not (“dark”). The percentage of PI positive
cells in the total amount of cells is shown.

| | | | | | Jurkat | |
|---|---|---|---|---|---|---|
| | light | | | | mean % | |
| No | conditions | | Compound | | cells PI+ | StDev |
| 1 | dark | | — | | 1.4 | 0.5 |
| 2 | dark | | I.1 50 | nM | 1.6 | 0.02 |
| 3 | dark | | I.1 100 | nM | 2.2 | 0.3 |
| 4 | dark | | I.1 200 | nM | 2.3 | 0.5 |
| 5 | dark | | I.1 400 | nM | 2.3 | 0.3 |
| 6 | dark | | I.1 600 | nM | 2.8 | 0.4 |
| 7 | dark | | CA4P 15 | nM | 8.0 | 1.6 |
| 8 | 390 | nm | — | | 1.1 | 0.08 |
| 9 | 390 | nm | I.1 50 | nM | 1.3 | 0.3 |
| 10 | 390 | nm | I.1 100 | nM | 2.3 | 0.2 |
| 11 | 390 | nm | I.1 200 | nM | 3.4 | 0.3 |
| 12 | 390 | nm | I.1 400 | nM | 6.3 | 0.3 |
| 13 | 390 | nm | I.1 600 | nM | 8.1 | 1.4 |
| 14 | 390 | nm | CA4P 15 | nM | 5.7 | 0.9 |

### Results 6: Nuclear Fragmentation

In order to determine whether cells treated with compound I.1 exhibit functional parameters of apoptosis, a quantification of nuclear fragmentation was performed according to the protocol established by Nicoletti^{[49]}. Different cell types, including MDA-MB-231, Jurkat, HeLa and HepG2 cells, were treated with indicated concentrations of compound I.1 and exposed to a 390 nm toxic regime, a rescue regime with both 390 nm and 515 nm light, or kept in the dark. Following the treatment, cells were stained with PI and the percentage of cells containing a hypodiploid amount of DNA (subG1 phase of cell cycle), was determined. Briefly, prior to analysis, cells were harvested on ice and incubated in a hypotonic buffer [0.1% sodium citrate, 0.1% Triton X-100 and 50 μg/mL propidium iodide (PI)] for 30 min at 4° C. Following the PI staining cells were analysed by flow cytometry using FACSCalibur flow cytometer (Becton Dickinson, Heidelberg, Germany) and Cell Quest Pro Software (Becton Dickinson). Subdiploid cells containing an amount of DNA inferior to that of the cells in the G1 phase were considered as apoptotic.

Results shown in Tables 13-17 represent means and standard deviations calculated for triplicates from one representative experiment out of three independent trials.

Compound I.1 showed similar behavior in a range of cell lines tested. Jurkat cells (Table 15) were most sensitive to compound I.1, while HepG2 cells showed higher resistance to this compound than HeLa cells, even when irradiated under more favourable conditions (Table 16), however all responded in the same qualitative way. This indicates that the invention's compounds have a generalizable mode of action as claimed for its mechanism of cytotoxicity.

EC₅₀ values (concentration required for a 50% increase of the subG1 population percentage from control conditions towards the plateau maximum value) were calculated for MDA-MB-231 cells treated with compound I.1 in two different lighting conditions. Compound I.1 exposed to the 390 nm regimen showed an EC₅₀ of approximately 1 μM, while in the dark it showed an EC₅₀ of approximately 120 μM (Tables 13 and 14). Moreover a reduction of the cytotoxic effect of compound I.1 (by comparison to the 390 nm regimen) was observed upon application of the rescue protocol (Table 17), similar to results reported in the context of cell cycle arrest. The results for control compound CA4P (Table 15) showed the same response regardless of the illumination protocol. Therefore taken together, the results demonstrate that the ability of compound I.1 to induce this key indicator of apoptosis can be fully reversibly controlled by light.

TABLE 13
Effect of compound I.1 on the induction of apoptosis in
MDA-MB-231 cells exposed to irradiation at 390 nm
(350 ms pulses every 5 min), or not (“dark”), for 48 h. The
mean percentage of cells in sub-G1 phase is shown.

| | | | MDA-MB-231 | | | |
|---|---|---|---|---|---|---|
| | | | DARK | | 390 nm | |
| | | | mean % | | mean % | |
| | | | subG1 | | subG1 | |
| No | Compound | | phase | StDev | phase | StDev |
| 1 | — | | 6.3 | 5.6 | 5.7 | 3.6 |
| 2 | I.1 10 | nM | 6.7 | 3.6 | 3.6 | 1.2 |
| 3 | I.1 100 | nM | 6.0 | 2.6 | 3.9 | 1.6 |
| 4 | I.1 400 | nM | 3.3 | 0.2 | 4.2 | 0.8 |
| 5 | I.1 600 | nM | 3.9 | 0.6 | 16.8 | 8.4 |
| 6 | I.1 800 | nM | 3.0 | 0.9 | 32.1 | 3.2 |
| 7 | I.1 1 | μM | 3.1 | 1.2 | 55.7 | 5.5 |
| 8 | I.1 1.2 | μM | 3.6 | 0.3 | 56.6 | 11.0 |
| 9 | I.1 1.5 | μM | 3.3 | 1.2 | 76.1 | 5.6 |
| 10 | I.1 3 | μM | 6.0 | 1.1 | 79.0 | 6.7 |
| 11 | I.1 6 | μM | 3.9 | 0.2 | 83.5 | 0.06 |
| 12 | I.1 15 | μM | 5.4 | 1.8 | 83.7 | 4.8 |

TABLE 14
Effect of high concentrations of
compound I.1 on the induction of
apoptosis in MDA-MB-231 cells kept
in the dark for 48 hours. The mean
percentage of cells in sub-G1
phase is shown.

| | | | MDA-MB-231 | |
|---|---|---|---|---|
| | | | DARK | |
| | | | mean % | |
| | | | subG1 | standard |
| No | Compound | | phase | deviation |
| 1 | — | | 1.6 | 0.2 |
| 2 | I.1 100 | nM | 2.1 | 0.6 |
| 3 | I.1 1 | μM | 2.0 | 0.6 |
| 4 | I.1 10 | μM | 2.0 | 0.2 |
| 5 | I.1 50 | μM | 4.5 | 1.1 |
| 6 | I.1 75 | μM | 7.9 | 1.2 |
| 7 | I.1 85 | μM | 12.7 | 3.5 |
| 8 | I.1 100 | μM | 26.8 | 6.1 |
| 9 | I.1 125 | μM | 51.1 | 25.5 |
| 10 | I.1 150 | μM | 92.2 | 4.9 |
| 11 | I.1 175 | μM | 95.7 | 0.5 |
| 12 | I.1 200 | μM | 95.5 | 0.2 |

TABLE 15
Effect of compound I.1 on the induction of
apoptosis in Jurkat cells exposed to irradiation
at 390 nm (350 ms pulses every 5 min) or not
(“dark”) for 48 h. The mean percentage
of cells in sub-G1 phase is shown.

| | | | Jurkat | | | |
|---|---|---|---|---|---|---|
| | | | DARK | | 390 nm | |
| | | | mean % | | mean % | |
| | | | subG1 | | subG1 | |
| No | Compound | | phase | StDev | phase | StDev |
| 1 | — | | 3.0 | 0.3 | 3.7 | 0.8 |
| 2 | I.1 5 | nM | 3.0 | 0.5 | 2.7 | 0.4 |
| 3 | I.1 50 | nM | 3.4 | 0.3 | 4.9 | 0.7 |
| 4 | I.1 100 | nM | 3.5 | 0.6 | 9.1 | 0.06 |
| 5 | I.1 150 | nM | 3.3 | 0.7 | 18.5 | 2.0 |
| 6 | I.1 200 | nM | 3.0 | 0.5 | 49.7 | 5.7 |
| 7 | I.1 400 | nM | 4.4 | 0.6 | 88.5 | 0.9 |
| 8 | I.1 800 | nM | 5.5 | 0.9 | 89.5 | 0.6 |
| 9 | I.1 1.5 | μM | 7.9 | 1.2 | 89.6 | 0.6 |
| 10 | I.1 5 | μM | 12.0 | 1.3 | 91.0 | 0.6 |
| 11 | CA4P 5 | nM | 37.5 | 2.2 | 40.4 | 1.0 |
| 12 | CA4P 15 | nM | 89.7 | 1.2 | 91.5 | 1.4 |

TABLE 16
Effect of compound I.1 on the induction of apoptosis in HeLa
and HepG2 cells. HeLa cells were kept in dark conditions
or exposed to 390 nm light (1 s pulses every 15 min) for
30 h, while HepG2 cells were kept in dark conditions or
exposed to 390 nm light (250 ms pulses every 15 s) for 48 h.
HeLa

| | | | | | mean % | |
|---|---|---|---|---|---|---|
| | light | | | | subG1 | |
| No | regime | | Compound | | phase | StDev |
| 1 | dark | | — | | 1.8 | 0.1 |
| 2 | dark | | I.1 200 | nM | 1.8 | 0.2 |
| 3 | dark | | I.1 600 | nM | 2.3 | 0.1 |
| 4 | dark | | I.1. 1.5 | μM | 2.9 | 0.01 |
| 5 | dark | | I.1 5 | μM | 3.2 | 0.1 |
| 6 | dark | | CA4P 15 | nM | 11.5 | 0.6 |
| 7 | 390 | nm | — | | 1.9 | 0.04 |
| 8 | 390 | nm | I.1 200 | nM | 2.4 | 0.2 |
| 9 | 390 | nm | I.1 600 | nM | 6.2 | 0.2 |
| 10 | 390 | nm | I.1 1.5 | μM | 16.1 | 0.9 |
| 11 | 390 | nm | I.1 5 | μM | 12.8 | 0.8 |
| 12 | 390 | nm | CA4P 15 | nM | 12.3 | 0.07 |

HepG2

| | | | | | mean % | |
|---|---|---|---|---|---|---|
| | light | | | | subG1 | |
| No | regime | | Compound | | phase | StDev |
| 1 | dark | | — | | 1.8 | 0.2 |
| 2 | dark | | I.1 100 | nM | 2.5 | 0.3 |
| 3 | dark | | I.1 600 | nM | 3.1 | 0.5 |
| 4 | dark | | I.1 1 | μM | 2.3 | 0.2 |
| 5 | dark | | I.1 3 | μM | 1.9 | 0.2 |
| 6 | dark | | I.1 10 | μM | 2.8 | 0.1 |
| 7 | 390 | nm | — | | 4.7 | 0.6 |
| 8 | 390 | nm | I.1 100 | nM | 5.4 | 0.9 |
| 9 | 390 | nm | I.1 600 | nM | 5.2 | 0.1 |
| 10 | 390 | nm | I.1 1 | μM | 4.6 | 1.2 |
| 11 | 390 | nm | I.1 3 | μM | 6.5 | 0.5 |
| 12 | 390 | nm | I.1 10 | μM | 14 | 2.1 |

TABLE 17
The “rescue” protocol of double illumination
reduced the induction of apoptosis in MDA-MB-231
cells relative to a 390 nm regimen. Cells were
illuminated for 48 h either with 390 nm
light only (pulses of 150 ms pulse every 30 s)
or in a rescue protocol where each such 390 nm pulse
was immediately followed by a pulse at 515 nm (500 ms).

| | | | | | 390 nm + | |
|---|---|---|---|---|---|---|
| | | | 390 nm | | 515 nm | |
| | | | mean | | mean | |
| | | | % | | % | |
| | | | subG1 | | subG1 | |
| No | Compound | | phase | StDev | phase | StDev |
| 1 | — | | 0.3 | 0.1 | 0.5 | 0.2 |
| 2 | I.1 10 | nM | 0.3 | 0.05 | 0.3 | 0.06 |
| 3 | I.1 100 | nM | 0.3 | 0.1 | 0.4 | 0.1 |
| 4 | I.1 400 | nM | 14.0 | 1.6 | 1.7 | 0.4 |
| 5 | I.1 600 | nM | 22.9 | 0.4 | 5.4 | 1.1 |
| 6 | I.1 800 | nM | 19.4 | 0.3 | 11.7 | 1.0 |
| 7 | I.1 1 | μM | 22.5 | 0.6 | 15.3 | 2.0 |
| 8 | I.1 2 | μM | 35.4 | 2.6 | 25.0 | 1.3 |

### Results 7: In Cellulo Tubulin Imaging

In order to investigate the influence of compound I.1 on microtubule structure in cellulo, immunostaining of the microtubule cytoskeleton in MDA-MB-231 cells was performed. Cells were treated with compound I.1 and then kept in dark conditions, or exposed to 390 nm light (200 ms every 2 min), or exposed to a rescue protocol with sequential pulses of 390 nm light (200 ms every 2 min) then immediately 505 nm (600 ms every 2 min). After 20 h of treatment cells were fixed, stained and subjected to confocal microscopy.

Briefly, prior to analysis, cells were washed with pre-warmed PBS then cell extraction buffer (80 mM piperazine-N,N′-bis(2-ethanesulfonic acid) [abbreviated PIPES], 1 mM MgCl₂, 5 mM EGTA-K and 0.5% Triton X-100, at pH 6.8) was added to remove monomeric and dimeric tubulin subunits. After 30 s of extraction, cells were fixed for 10 min with 0.5% glutaraldehyde. Following glutaraldehyde quenching with 0.1% NaBH₄ in PBS for 7 min, coverslips were blocked with PBS containing 0.2% BSA (bovine serum albumin). Immunostaining was performed using anti-α-tubulin antibody (ab18251) and the AlexaFluor 488 secondary antibody (A 11008), purchased from Abcam (Cambridge, UK) and Invitrogen (Darmstadt, Germany), respectively. Hoechst 33342 (bisbenzimide), purchased from Sigma-Aldrich (catalogue number B2261; Taufkirchen, Germany), was used at a concentration of 1 ug/ml for nuclear staining. Cells were mounted with PermaFluor™ mounting medium (Beckman Coulter) and analyzed with a Zeiss LSM 510 Meta confocal microscope (Jena, Germany). Acquired images were processed using the ImageJ program (National Institutes of Health) and representative images are collected in FIG. 7.

Non-treated cells showed intact, long and polarized microtubules. In the 390 nm regimen, treatment with 1.5 μM of compound I.1 led to degradation of the microtubule cytoskeleton, and 4 μM of compound I.1 led to complete microtubule breakdown plus fragmentation of the nuclei (typical for apoptotic cells). Such nuclear fragmentation and microtubule destruction were not observed in control cells treated with compound I.1 but kept in the dark, indicating the light-controlled toxic effect of compound I.1. The rescue protocol also led to a dose-dependent reduction of the signs of cytotoxic effects of compound I.1, indicating the reversible photo-control of the tubulin polymerisation inhibitor properties of compound I.1 in cellulo.

### BIBLIOGRAPHY

[1] G. C. Tron, T. Pirali, G. Sorba, F. Pagliai, S. Busacca, A. A. Genazzani, J. Med. Chem. 2006, 49, 3033.
[2] G. M. Tozer, C. Kanthou, C. S. Parkins, S. A. Hill, Int J Exp Pathol 2002, 83, 21.
[3] M. Cushman, D. Nagarathnam, D. Gopal, H. M. He, C. M. Lin, E. Hamel, J. Med. Chem. 1992, 35, 2293.
[4] K. Ohsumi, R. Nakagawa, Y. Fukuda, T. Hatanaka, Y. Morinaga, Y. Nihei, K. Ohishi, Y. Suga, Y. Akiyama, T. Tsuji, J. Med. Chem. 1998, 41, 3022.
[5] K. Ohsumi, T. Hatanaka, K. Fujita, R. Nakagawa, Y. Fukuda, Y. Nihei, Y. Suga, Y. Morinaga, Y. Akiyama, T. Tsuji, Bioorg. Med. Chem. Lett. 1998, 8, 3153; N. Mur Blanch, G. G. Chabot, L. Quentin, D. Scherman, S. Bourg, D. Dauzonne, Eur. J. Med. Chem. 2012, 54, 22.
[6] R. A. Stanton, K. M. Gernert, J. H. Nettles, R. Aneja, Medicinal Research Reviews 2011, 31, 443.
[7] K. Helmut, in CRC Handbook of Organic Photochemistry and Photobiology, Volumes 1 & 2, Second Edition (Ed.: J. F. Rabek), CRC Press, 2003.
[8] J. Hadfield, A. McGown, S. Mayalarp, E. Land, I. Hamblett, K. Gaukroger, N. Lawrence, L. Hepworth, J. Butler, US2013023663, 2013.
[9] R. Bisby, S. Botchway, J. Hadfield, A. McGown, K. M. Scherer, WO2013021208, 2013.
[10] L. Grossweiner, The Science of Phototherapy: An Introduction, Springer Netherlands, 2005.
[11] F. B. Mallory, C. W. Mallory, in Organic Reactions, John Wiley & Sons, Inc., 2004.
[12] M. Smith, J. March, March's Advanced Organic Chemistry, Sixth Edition ed., John Wiley & Sons, Hoboken, N.J., 2007.
[13] 0. M. Friedman, R. M. Gofstein, A. M. Seligman, J. Am. Chem. Soc. 1949, 71, 3010.
[14] A. M. Seligman, R. M. Gofstein, O. M. Friedman, Cancer 1952, 5, 613.
[15] F. M. H. de Groot, L. W. A. van Berkom, H. W. Scheeren, J. Med. Chem. 2000, 43, 3093.
[16] R. Hewitt, K. Dano, Enzyme Protein 1996, 49, 163.
[17] L. D. Lavis, R. T. Raines, ACS Chem. Biol. 2014.
[18] M. Bio, P. Rajaputra, G. Nkepang, S. G. Awuah, A. M. L. Hossion, Y. You, J. Med. Chem. 2013, 56, 3936; E. Escalona-Benz, M. E. Jockovich, T. G. Murray, B. Hayden, E. Hernandez, W. Feuer, J. J. Windle, Investigative Ophthalmology & Visual Science 2005, 46, 8; P. Thomson, M. A. Naylor, S. A. Everett, M. R. L. Stratford, G. Lewis, S. Hill, K. B. Patel, P. Wardman, P. D. Davis, Molecular Cancer Therapeutics 2006, 5, 2886.
[19] G. R. Pettit, M. R. Rhodes, US20097557096, 2009.
[20] R. J. Amir, D. Shabat, Chem. Commun. 2004, 1614; D. Shabat, J. Polym. Sci., Part A: Polym. Chem. 2006, 44, 1569; D. Shabat, R. J. Amir, A. Gopin, N. Pessah, M. Shamis, Chem.-Eur. J 2004, 10, 2626.
[21] W. S. Saari, J. E. Schwering, P. A. Lyle, S. J. Smith, E. L. Engelhardt, J. Med. Chem. 1990, 33, 97; M. A. DeWit, E. R. Gillies, Org. Biomol. Chem. 2011, 9, 1846.
[22] O. Thorn-Seshold, M. Vargas-Sanchez, S. McKeon, J. Hasserodt, Chem. Commun. 2012, 48, 6253.
[23] M. H. Lee, J. H. Han, J.-H. Lee, H. G. Choi, C. Kang, J. S. Kim, J. Am. Chem. Soc. 2012, 134, 17314; M. A. Dewit, A. Beaton, E. R. Gillies, J. Polym. Sci., Part A: Polym. Chem. 2010, 48, 3977.
[24] M. N. Levine, R. T. Raines, Chem. Sci. 2012, 3, 2412; M. N. Levine, R. T. Raines, Anal. Biochem. 2011, 418, 247; S. S. Chandran, K. A. Dickson, R. T. Raines, J. Am. Chem. Soc. 2005, 127, 1652.
[25] L. Zhang, D. Duan, Y. Liu, C. Ge, X. Cui, J. Sun, J. Fang, J. Am. Chem. Soc. 2013.
[26] M. Freccero, Mini-Rev. Org. Chem, 2004, 1, 403; D. H. Kwan, H.-M. Chen, K. Ratananikom, S. M. Hancock, Y. Watanabe, P. T. Kongsaeree, A. L. Samuels, S. G. Withers, Angew. Chem., Int. Ed. 2011, 50, 300; S. E. Rokita, J. Yang, P. Pande, W. A. Greenberg, J. Org. Chem. 1997, 62, 3010; J. D. Sellars, M. Landrum, A. Congreve, D. P. Dixon, J. A. Mosely, A. Beeby, R. Edwards, P. G. Steel, Org. Biomol. Chem. 2010, 8, 1610; E. E. Weinert, R. Dondi, S. Colloredo-Melz, K. N. Frankenfield, C. H. Mitchell, M. Freccero, S. E. Rokita, J. Am. Chem. Soc. 2006, 128, 11940; Z. Zhang, K. Tanabe, H. Hatta, S.-i. Nishimoto, Org. Biomol. Chem. 2005, 3, 1905.
[27] P. Wang, Asian Journal of Organic Chemistry 2013, 2, 452.
[28] G. R. Pettit, C. Temple, V. L. Narayanan, R. Varma, M. J. Simpson, M. R. Boyd, G. A. Rener, N. Bansal, Anti-Cancer Drug Des. 1995, 10, 299.
[29] C. M. Lin, S. B. Singh, P. S. Chu, R. O. Dempcy, J. M. Schmidt, G. R. Pettit, E. Hamel, Molecular Pharmacology 1988, 34, 200.
[30] M. Cushman, D. Nagarathnam, D. Gopal, A. K. Chakraborti, C. M. Lin, E. Hamel, J. Med. Chem. 1991, 34, 2579.
[31] K. Saotome, H. Morita, M. Umeda, Toxicology in Vitro 1989, 3, 317.
[32] A. A. Beharry, G. A. Woolley, Chem. Soc. Rev. 2011, 40, 4422.
[33] D. Shcherbakov, K. Motovilov, I. Erofeev, A. Astafiev, ArXiv e-prints 2014, 1401.0346.
[34] J. P. Krise, R. Oliyai, in Prodrugs, Vol. 5, Springer, New York, 2007, pp. 801; S. Papot, I. Tranoy, F. Tillequin, J. C. Florent, J. P. Gesson, Current Medicinal Chemistry—Anti-Cancer Agents 2002, 2, 155; M. Rooseboom, J. N. M. Commandeur, N. P. E. Vermeulen, Pharmacological Reviews 2004, 56, 53; B. Seddon, L. R. Kelland, P. Workman, in Suicide Gene Therapy: Methods and Reviews (Ed.: C. Springer), Springer, 2003, pp. 515; V. Stella, S. Dhareshwar, in Prodrugs: Challenges and Rewards, Springer, New York, 2007, pp. 731.
[35] D. Bléger, J. Schwarz, A. M. Brouwer, S. Hecht, J. Am. Chem. Soc. 2012, 134, 20597.
[36] J. Griggs, J. N. Skepper, G. A. Smith, K. M. Brindle, J. C. Metcalfe, R. Hesketh, Am J Pathol 2002, 160, 1097.
[37] C. Conde, A. Caceres, Nat. Rev. Neurosci. 2009, 10, 319; S. K. Dutcher, Curr. Opin. Cell Biol. 2001, 13, 49.
[38] A. A. Beharry, G. A. Woolley, in Photosensitive Molecules for Controlling Biological Function, 2011, pp. 171.
[39] E. Merino, Chem. Soc. Rev. 2011, 40, 3835.
[40] T. W. Greene, P. G. M. Wuts, Protective Groups in Organic Synthesis, Third ed., John Wiley & Sons, Inc., 2002.
[41] Kocienski, Protecting Groups 3rd ed., Thieme Verlag, 2005.
[42] H. E. Gottlieb, V. Kotlyar, A. Nudelman, J. Org. Chem. 1997, 62, 7512.
[43] E. L. Bastos, L. F. M. L. Ciscato, W. J. Baader, Synth. Commun. 2005, 35, 1501.
[44] K. Bailey, R. Cowling, E. W. Tan, D. Webb, Bioorg. Med. Chem. Lett. 2004, 12, 595.
[45] K. Shindo, R. Nakamura, I. Chinda, Y. Ohnishi, S. Horinouchi, H. Takahashi, K. Iguchi, S. Harayama, K. Furukawa, N. Misawa, Tetrahedron 2003, 59, 1895.
[46] T. Nguyen, M. B. Francis, Org. Lett. 2003, 5, 3245.
[47] E. Boonacker, C. J. F. Van Noorden, J. Histo. Cyto. 2001, 49, 1473; S. Ibsen, E. Zahavy, W. Wrasidlo, T. Hayashi, J. Norton, Y. Su, S. Adams, S. Esener, Photochem. Photobiol. 2013, 89, 698.
[48] A. A. Beharry, O. Sadovski, G. A. Woolley, J. Am. Chem. Soc. 2011, 133, 19684.
[49] I. Nicoletti, G. Migliorati, M. C. Pagliacci, F. Grignani, C. Riccardi, J Immunol Methods 1991, 139, 271.

1-55. (canceled)

56. Compounds corresponding to one of the following formulae: 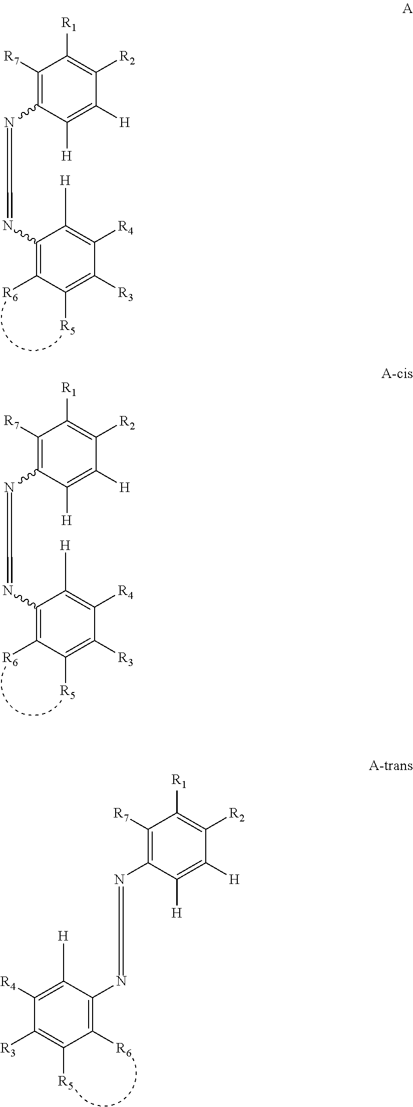
wherein:
the aryl ring bearing R₃ is denoted the “south ring”,
dotted lines indicate sites where a fused ring may be present;
R₂ is chosen among —OCH₃, —OCF₃, —F, —CH₃, —CF₃, —CH₂CH₃, —OCH₂CH₃, —SCH₃, —SCF₃, NHCH₃, —N(CH₃)₂ and —CN; and
R₁, R₆ and R₇ are defined as follows:
R₁ is chosen among hydrogen, —Y₁Rₐ, —S₂R_{b}, —NHR_{d}, —ORₑ, —OPO₃H₂, —NO₂, —B(OH)₂, —B(OR_{b})₂, —B(OR_{b}O), —N₃, —F, —Cl, —Br, —I, —CHO, —CO₂H, —CONH₂, —CN, —NC, —SO₃H, —CO₂R_{b}, —SO₂NH₂ and —R_{b};
R₇, and when R₆ is not linked to R₅, R₆ also, identical or different, are chosen among hydrogen, —Y₂R_{f}, —S₂R_{g}, —NHRᵢ, —ORⱼ, —OPO₃H₂, —NO₂, —B(OH)₂, —B(OR_{g})₂, —B(OR_{g}O), —N₃, —F, —Cl, —Br, —I, —CHO, —CO₂H, —CONH₂, —CN, —NC, —SO₃H, —CO₂R_{g}, —SO₂NH₂, —R_{g}, —CO₂NHR_{g}, —CO₂NR_{g}Rₕ, —N-piperazinyl, —N-morpholinyl, —N-pyrrolidinyl, —N-piperidinyl, and -linker-reporter units; and when R₆ is linked to R₅ then they are linked together forming a fused phenyl, 2-pyridinyl or 3-pyridinyl ring, the said phenyl, 2-pyridinyl or 3-pyridinyl being unsubstituted or substituted with one or several groups Rₙ, identical or different, such that the south ring is respectively a naphthalene, quinoline or isoquinoline;
considering that at least one of the substituents R₆, R₇ and R₁ is different from hydrogen; and
R₃ is chosen among —OCH₃, —OCF₃, —F, —CH₃, —CF₃, —CH₂CH₃, —OCH₂CH₃, —SCH₃, —SCF₃, —NHCH₃, —N(CH₃)₂ and —CN;
R₄ and, when R₆ is not linked to R₅, R₅ also, identical or different, are chosen among —OCH₃, —OCF₃, —F, —CH₃, —CF₃, —CH₂CH₃, —OCH₂CH₃, —SCH₃, —SCF₃, —NH₂, —NHCH₃, —N(CH₃)₂ and —CN;
Y₁═O, S, NH or NRₖ;
Y₂═O, S, NH or NR₁;
Rₐ is chosen among hydrogen, —R_{b}, —COR_{b}, —CO₂R_{b}, —CONH₂, —CONR_{b}R_{c}, —CONHR_{b}, —CH₂OC(O)R_{b}, and cleavable groups which after cleavage, for instance in vivo, lead either to R₁═—OH when Y₁═O, or to R₁═—NH₂ when Y₁═NH, or to R₁═—NHRₖ when Y₁═NRₖ, or to R₁═—SH when Y₁═S;
R_{b}, R_{c}, R_{g}, Rₕ, Rₖ and Rₗ, identical or different, are chosen among (C₁-C₆)alkyl, (C₁-C₆)alkyl-OH, (C₁-C₆)alkenyl, (C₁-C₆)alkynyl, (C₃-C₇)cycloalkyl, aryl, heteroaryl, heterocycle, (C₁-C₆)alkyl(C₃-C₇)cycloalkyl, (C₁-C₆)alkylaryl, (C₁-C₆)alkylheteroaryl, and (C₁-C₆)alkylheterocycle;
R_{d} and Rᵢ are identical or different, and are a peptidic group attached via its carboxyl terminus;
Rₑ and Rⱼ are identical or different, and are a glycosidyl group;
R_{f} is chosen among hydrogen, —R_{g}, —COR_{g}, —CO₂R_{g}, —CONH₂, —CONR_{g}Rₕ, —CONHR_{g}, —OCH₂OC(O)R_{g}, and cleavable groups which after, for instance in vivo, lead either to R₆ or R₇═—OH when Y₂═O, to R₆ or R₇═—NH₂ when Y₂═NH, or to R₆ or R₇═—NHR₁ when Y₂═NR₁, or to R₆ or R₇═—SH when Y₂═S;
Rₙ is chosen among —CH₃, —OH, —NH₂, —NHCOCH₃, —SO₃H, —CO₂H, —CONH₂, —CO₂CH₃, —PO₃H₂, —NO₂, —B(OH)₂, —N₃, —CN, —C≡CH, and —SO₂NH₂;
and their hydrates, pharmaceutically acceptable salts and solvates, as a mixture of isomers in any proportions and also as pure isomer.

57. Compounds according to claim 56, wherein R₁═Y₁Rₐ, with Y₁═O, NH or S and Rₐ being as defined in claim 56.

58. Compounds according to claim 56, wherein R₁ is chosen among —OH, —NH₂ or —SH.

59. Compounds according to claim 56, wherein R₇═—Y₂R_{f}, Y₂═O, NH or S and R_{f} is as defined in claim 1 and/or R₆═—Y₂R_{f}, Y₂═O, NH or S, and R_{f} is as defined in claim 56.

60. Compounds according to claim 56, wherein R₁═—NH-peptidic group and/or R₇═—NH-peptidic group and/or R₆ is a —NH-peptidic group.

61. Compounds according to claim 56, wherein R₆ is a linker-reporter unit -Link1-Rep1 and/or R₇ is a linker-reporter unit -Link2-Rep2 where:
the reporter Rep1 and Rep2, identical or different, are chosen among fluorophores, chromophores, antennas and tag moieties, and especially among fluorescein, rhodamine, coumarin, phenoxazine, acridine, boron-dipyrromethene, dansyl, propidium, nitrobenzofurazan, resorufin, cyanine, Cascade Yellow, Nile Red, carborhodamine, silarhodamine, DABCYL, black hole quencher moieties, (E)-4,4′-bis(diethylamino)stilbene, biotin, and tag protein substrates, especially O⁶-benzylguanine, O²-benzylcytosine or —((CH₂)₂O)₂(CH₂)₆Cl, and their derivatives, and
the linker Link1 and Link2, identical or different, are chosen among bivalent (C₁-C₁₂)alkyl; bivalent (C₁-C₁₂)alkenyl; —(CH₂)ₘ₁(C₃-C₇)cycloalkyl(CH₂)ₘ₂—, —(CH₂)ₘ₁aryl(CH₂)ₘ₂—; moieties including between 1 to 10 carbon atoms and 1 to 6 heteroatoms chosen from among oxygen, nitrogen and sulfur, such as —(CH₂)ₘ₁heteroaryl(CH₂)ₘ₂— especially when heteroaryl is a triazole, tetrazole or pyridazine, —(CH₂)ₘ₁heterocycle(CH₂)ₘ₂—, oligo(ethyleneglycol), —(CH₂)ₘ₁—C(O)O—(CH₂)ₘ₂—, —(CH₂)ₘ₁—C(O)NH—(CH₂)ₘ₂—, —C(O)—, —(CH₂)ₘ₁—S—S—(CH₂)ₘ₂—, —(CH₂)ₘ₁—N-succinimide-3-S(CH₂)ₘ₂—, —C(O)-(4-cyclohexyl)-CH₂—N-succinimide-3-S(CH₂)ₘ₂— and —(CH₂)ₘ₁—S—CH₂C(O)—(CH₂)ₘ₂— with m1 and m2, identical or different, being integers chosen in the range 0 to 6.

62. Compounds according to claim 56, wherein R₆ and R₇ are chosen among —H, —F, —Cl, —NO₂, —NHCOCH₃, —N(CH₃)₂, and —OCH₃.

63. Compounds according to claim 56, wherein R₆═H.

64. Compounds according to claim 56, wherein R₂ and R₃ are chosen separately among —OCH₃, —OCF₃, —F, —CH₃, —CF₃, —CH₂CH₃ and —OCH₂CH₃.

65. Compounds according to claim 56, wherein R₂, R₃, R₄, and R₅ are chosen separately among —OCH₃, —OCF₃, —F, —CH₃, —CF₃, —CH₂CH₃ and —OCH₂CH₃.

66. Compounds according to claim 56, wherein R₂═R₃═R₄═R₅═OCH₃.

67. Compounds according to claim 56, wherein R₂═R₃═R₄═R₅═OCH₃; R₆ and R₇ are chosen among —H, —F, —Cl, —NO₂, —NHCOCH₃, —N(CH₃)₂, and —OCH₃.

68. Compounds according to claim 56 chosen among:
2-methoxy-5-((3,4,5-trimethoxyphenyl)diazenyl)phenol (I.1): 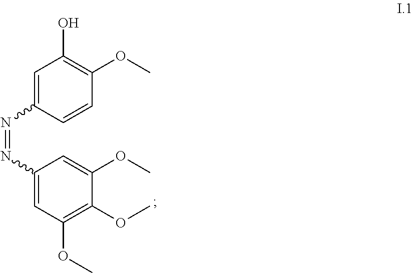 [SMILES: COc1ccc(N=Nc2cc(OC)c(OC)c(OC)c2)cc1O]
2-methoxy-5-((3,4,5-trimethoxyphenyl)diazenyl)aniline (I.2): 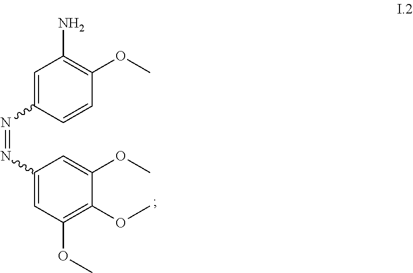 [SMILES: COc1ccc(N=Nc2cc(OC)c(OC)c(OC)c2)cc1N]
1-(3-fluoro-4-methoxyphenyl)-2-(3,4,5-trimethoxyphenyl)diazene (I.3): 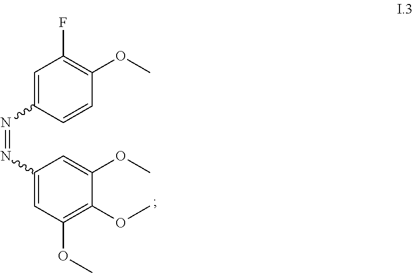 [SMILES: COc1ccc(N=Nc2cc(OC)c(OC)c(OC)c2)cc1F]
1-(2-fluoro-4-methoxyphenyl)-2-(3,4,5-trimethoxyphenyl)diazene (I.4): 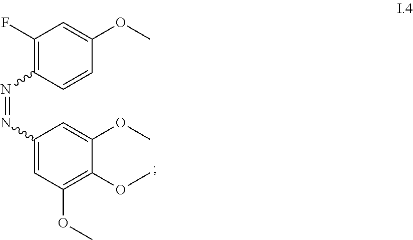 [SMILES: COc1ccc(N=Nc2cc(OC)c(OC)c(OC)c2)c(F)c1]
1-(2,3-difluoro-4-methoxyphenyl)-2-(3,4,5-trimethoxyphenyl)diazene (I.5): 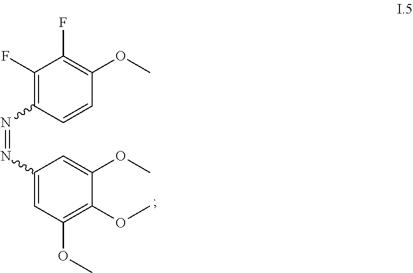 [SMILES: COc1ccc(N=Nc2cc(OC)c(OC)c(OC)c2)c(F)c1F]
1-(2-fluoro-4-methoxy-3-nitrophenyl)-2-(3,4,5-trimethoxyphenyl)diazene (I.7): 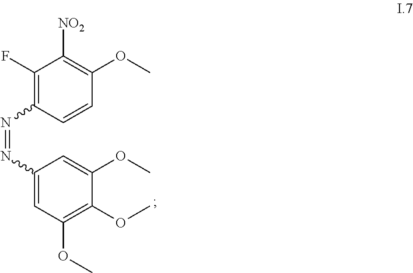 [SMILES: COc1cc(N=Nc2ccc(OC)c([N+](=O)[O-])c2F)cc(OC)c1OC]
5-methoxy-2-((3,4,5-trimethoxyphenyl)diazenyl)phenol (I.8): 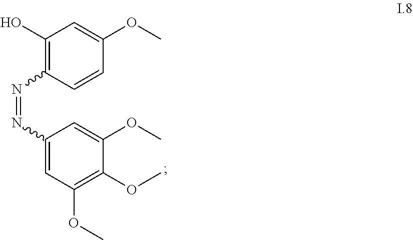 [SMILES: COc1ccc(N=Nc2cc(OC)c(OC)c(OC)c2)c(O)c1]
(2-methoxy 5-((3,4,5-trimethoxyphenyl)diazenyl)phenyl) 2-((L-leucinamido)methyl)piperidine 1-carboxylate 2,2,2-trifluoroacetate salt (I.10): 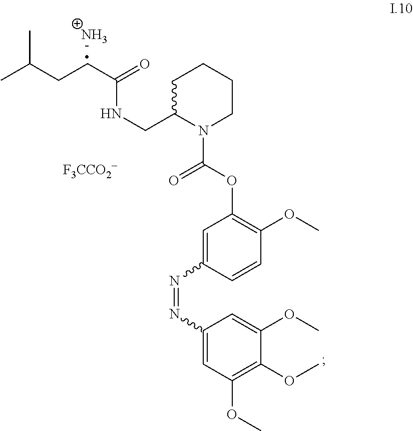
and its free form,
1-(3,4-dimethoxyphenyl)-2-(3,4,5-trimethoxyphenyl)diazene (I.11): 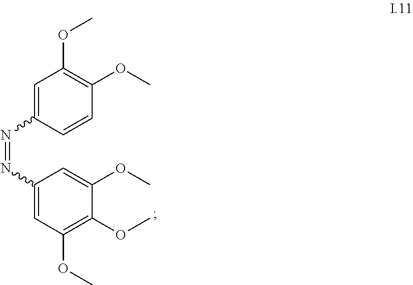 [SMILES: COc1ccc(N=Nc2cc(OC)c(OC)c(OC)c2)cc1OC]
1-(2,4-dimethoxyphenyl)-2-(3,4,5-trimethoxyphenyl)diazene (I.12): 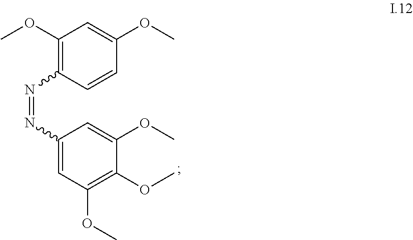
5-methoxy-2-((3,4,5-trimethoxyphenyl)diazenyl)aniline (I.16): 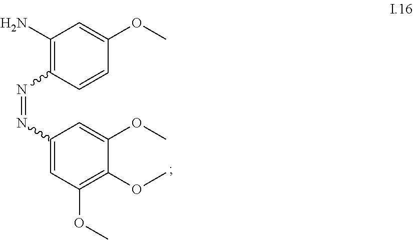 [SMILES: COc1ccc(N=Nc2cc(OC)c(OC)c(OC)c2)c(N)c1]
3-methoxy-2-methyl-6-((3,4,5-trimethoxyphenyl)diazenyl)phenol (I.17): 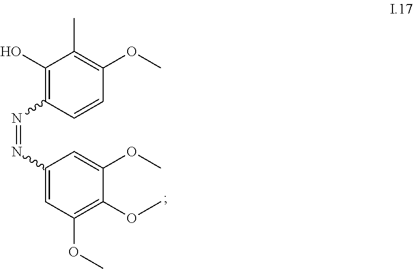 [SMILES: COc1ccc(N=Nc2cc(OC)c(OC)c(OC)c2)c(O)c1C]
2-(3-methoxy-2-methyl-6-((3,4,5-trimethoxyphenyl)diazenyl)phenoxy)ethan-1-ol (I.18): 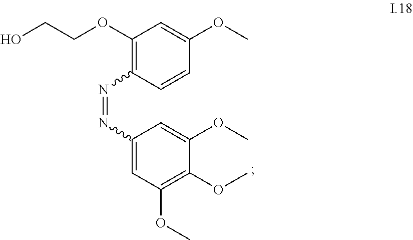 [SMILES: COc1ccc(N=Nc2cc(OC)c(OC)c(OC)c2)c(OCCO)c1]
2-(5-methoxy-2-((3,4,5-trimethoxyphenyl)diazenyl)phenoxy)ethan-1-ol (I.19): 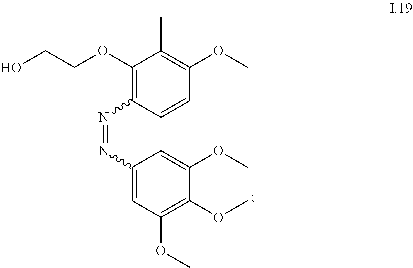 [SMILES: COc1ccc(N=Nc2cc(OC)c(OC)c(OC)c2)c(OCCO)c1C]
(2-methoxy 5-((3,4,5-trimethoxyphenyl)diazenyl)phenyl)) 1-L-serinamide 2,2,2-trifluoroacetate salt (I.20): 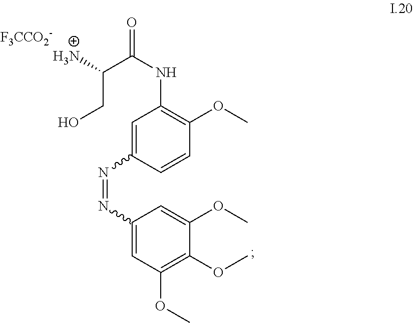 [SMILES: "F3CCO2-" ; COc1ccc(N=Nc2cc(OC)c(OC)c(OC)c2)cc1NC(=O)[C@@H]([NH3+])CO]
and its free form,
3-(3-methoxy-2-methyl-6-((3,4,5-trimethoxyphenyl)diazenyl)phenoxy)propan-1-ol (I.21): 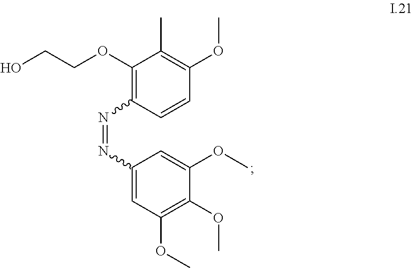 [SMILES: COc1ccc(N=Nc2cc(OC)c(OC)c(OC)c2)c(OCCO)c1C]
2-methoxy-5-((3,4,5-trimethoxyphenyl)diazenyl)phenyl phosphate disodium salt (I.24): 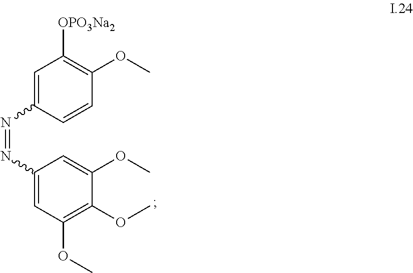
and its free form;
N-(6-(diethylamino)-9-(2-(4-(3-(3-methoxy-2-methyl-6-((3,4,5-trimethoxyphenyl)diazenyl)phenoxy)propyl)piperazine-1-carbonyl)phenyl)-3H-xanthen-3-ylidene)-N-ethylethanaminium bis(formate) salt (I.25): 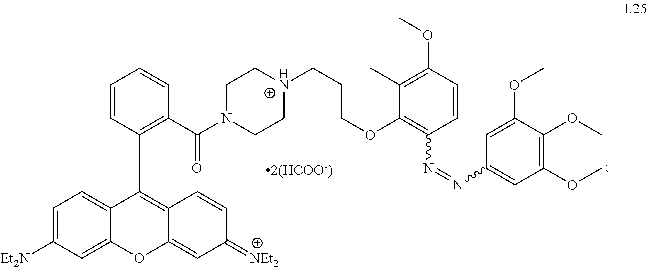
N-(5-methoxy-2-((3,4,5-trimethoxyphenyl)diazenyl)phenyl)acetamide (I.26): 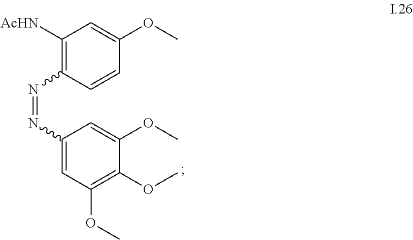 [SMILES: COc1ccc(N=Nc2cc(OC)c(OC)c(OC)c2)c(NC(C)=O)c1]
1-(3-bromo-4-methoxyphenyl)-2-(3,4,5-trimethoxyphenyl)diazene (I.27): 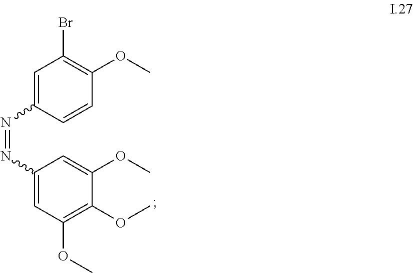 [SMILES: COc1ccc(N=Nc2cc(OC)c(OC)c(OC)c2)cc1Br]
2-methoxy-5-((3,4,5-trimethoxyphenyl)diazenyl)benzaldehyde (I.28): 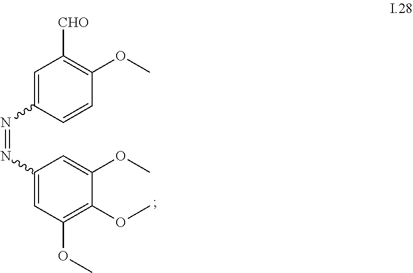 [SMILES: COc1ccc(N=Nc2cc(OC)c(OC)c(OC)c2)cc1C=O]
and 2-methoxy-5-((3,4,5-trimethoxyphenyl)diazenyl)benzoic acid (I.29): 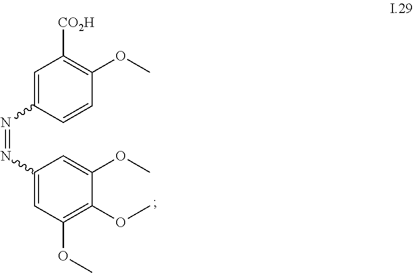 [SMILES: COc1ccc(N=Nc2cc(OC)c(OC)c(OC)c2)cc1C(=O)O]
as a mixture of cis and trans isomers in any proportions and also as a pure isomer either cis or trans, and their hydrates, pharmaceutically acceptable salts and solvates.

69. Compounds according to claim 56 chosen among: 5-((3,5-dimethoxy-4-(trifluoromethoxy)phenyl)diazenyl)-2-(trifluoromethoxy)phenol; 2-(trifluoromethoxy)-5-((3,4,5-trimethoxyphenyl)diazenyl)phenol; 5-((3,5-dimethoxy-4-(trifluoromethoxy)phenyl)diazenyl)-2-methoxyphenol; 2-fluoro-6-methoxy-3-((3,4,5-trimethoxyphenyl)diazenyl)aniline; N-(2-hydroxy-3-methoxy-6-((3,4,5-trimethoxyphenyl)diazenyl)phenyl)acetamide; 5-((3,5-dimethoxy-4-(trifluoromethoxy)phenyl)diazenyl)-2-(trifluoromethoxy)phenyl dihydrogen phosphate; 2-(trifluoromethoxy)-5-((3,4,5-trimethoxyphenyl)diazenyl)phenyl dihydrogen phosphate and 5-((3,5-dimethoxy-4-(trifluoromethoxy)phenyl)diazenyl)-2-methoxyphenyl dihydrogen phosphate; as a mixture of cis and trans isomers in any proportions and also as a pure isomer either cis or trans, and their hydrates, pharmaceutically acceptable salts and solvates.

70. Compounds according to claim 56 for their use as a medicament, and in particular as an anti-mitotic, anti-angiogenic, antitumoral or chemotherapeutic agent.

71. Compounds according to claim 56 for their use in the treatment of a disease for which the administration of a compound with antitubulin activity has a beneficial effect.

72. Compounds according to claim 56 for their use in the treatment of a cancer, such as melanoma, adenocarcinoma of the lung, neuroblastoma, small cell carcinoma of the lung, breast carcinoma, colon carcinoma, ovarian carcinoma, or bladder carcinoma, or of a disease characterized by abnormal vascularisation such as diabetic retinopathy, psoriasis or endometriosis, or of rheumatoid arthritis or atherosclerosis.

73. A pharmaceutical composition comprising a compound according to claim 56 with at least one pharmaceutically acceptable excipient.

74. A compound with an azoaryl structure for use in the treatment of a disease for which a tubulin polymerisation inhibitor activity has a beneficial effect, wherein the compound is administered to the patient in need of such treatment, at least partially in its trans isomeric form of the diazenyl bond, and where this trans form is inactive as regards a tubulin polymerisation inhibitory effect, and where the compound is photoisomerised in vivo to an azoaryl compound in its cis isomeric form of the diazenyl bond by the application of light, with optional modification in vivo of one or more substituents either before or after this photoisomerisation, and where this resultant cis form is active as regards a tubulin polymerisation inhibitory effect.

75. A compound for use according to claim 74, wherein the azoaryl compound is administered:
in its trans isomeric form of the diazenyl bond, and where its cis form is active as regards a tubulin polymerisation inhibitory effect;
or in a mixture of cis and trans isomeric forms of the diazenyl bond, and where its cis form is active as regards a tubulin polymerisation inhibitory effect.

76. A compound for use according to claim 74, wherein the application of light is localized.

77. A compound for use according to claim 74, wherein the isomerisation in vivo of the diazenyl bond from trans to cis form is followed by a conversion of cis to trans form by spontaneous thermal reversion or by application of light.

78. A compound for use according to claim 77, wherein the isomerisation in vivo of the diazenyl bond from trans to cis form leads to an inactive form as regards a tubulin polymerisation inhibitory effect.

79. A compound for use according to claim 74, wherein the disease is a cancer, such as melanoma, adenocarcinoma of the lung, neuroblastoma, small cell carcinoma of the lung, breast carcinoma, colon carcinoma, ovarian carcinoma, or bladder carcinoma, or a disease characterized by abnormal vascularisation such as diabetic retinopathy, psoriasis or endometriosis, or rheumatoid arthritis or atherosclerosis.

80. A compound for use according to claim 74, wherein the compound is selected among the compounds: 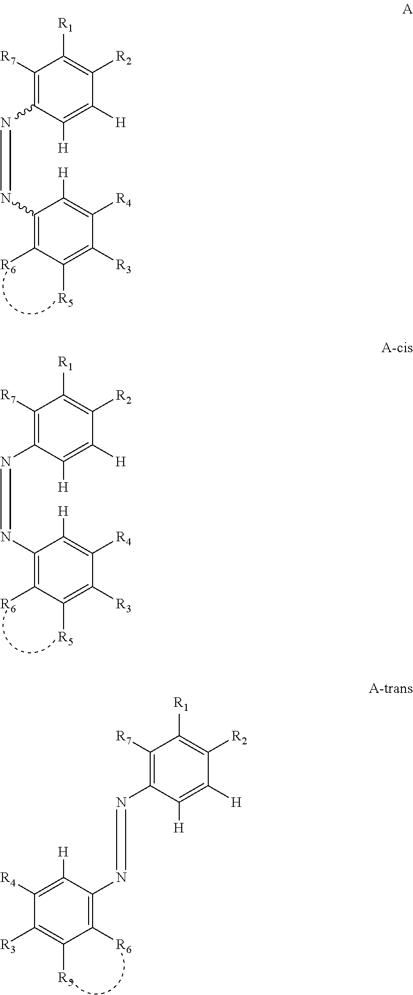
wherein:
the aryl ring bearing R₃ is denoted the “south ring”,
dotted lines indicate sites where a fused ring may be present;
R₂ is chosen among —OCH₃, —OCF₃, —F, —CH₃, —CF₃, —CH₂CH₃, —OCH₂CH₃, —SCH₃, —SCF₃, NHCH₃, —N(CH₃)₂ and —CN; and
R₁, R₆ and R₇ are defined as follows:
R₁ is chosen among hydrogen, —Y₁Rₐ, —S₂R_{b}, —NHR_{d}, —ORₑ, —OPO₃H₂, —NO₂, —B(OH)₂, —B(OR_{b})₂, —B(OR_{b}O), —N₃, —F, —Cl, —Br, —I, —CHO, —CO₂H, —CONH₂, —CN, —NC, —SO₃H, —CO₂R_{b}, —SO₂NH₂ and —R_{b};
R₇, and when R₆ is not linked to R₅, R₆ also, identical or different, are chosen among hydrogen, —Y₂R_{f}, —S₂R_{g}, —NHR₁, —ORⱼ, —OPO₃H₂, —NO₂, —B(OH)₂, —B(OR_{g})₂, —B(OR_{g}O), —N₃, —F, —Cl, —Br, —I, —CHO, —CO₂H, —CONH₂, —CN, —NC, —SO₃H, —CO₂R_{g}, —SO₂NH₂, —R_{g}, —CO₂NHR_{g}, —CO₂NR_{g}Rₕ, —N-piperazinyl, —N-morpholinyl, —N-pyrrolidinyl, —N-piperidinyl, and -linker-reporter units; and when R₆ is linked to R₅ then they are linked together forming a fused phenyl, 2-pyridinyl or 3-pyridinyl ring, the said phenyl, 2-pyridinyl or 3-pyridinyl being unsubstituted or substituted with one or several groups Rₙ, identical or different, such that the south ring is respectively a naphthalene, quinoline or isoquinoline;
considering that at least one of the substituents R₆, R₇ and R₁ is different from hydrogen; and
R₃ is chosen among —OCH₃, —OCF₃, —F, —CH₃, —CF₃, —CH₂CH₃, —OCH₂CH₃, —SCH₃, —SCF₃, —NHCH₃, —N(CH₃)₂ and —CN;
R₄ and, when R₆ is not linked to R₅, R₅ also, identical or different, are chosen among —OCH₃, —OCF₃, —F, —CH₃, —CF₃, —CH₂CH₃, —OCH₂CH₃, —SCH₃, —SCF₃, —NH₂, —NHCH₃, —N(CH₃)₂ and —CN;
Y₁═O, S, NH or NRₖ;
Y₂═O, S, NH or NRₗ;
Rₐ is chosen among hydrogen, —R_{b}, —COR_{b}, —CO₂R_{b}, —CONH₂, —CONR_{b}R_{c}, —CONHR_{b}, —CH₂OC(O)R_{b}, and cleavable groups which after cleavage, for instance in vivo, lead either to R₁═—OH when Y₁═O, or to R₁═—NH₂ when Y₁═NH, or to R₁═—NHRₖ when Y₁═NRₖ, or to R₁═—SH when Y₁═S;
R_{b}, R_{c}, R_{g}, Rₕ, Rₖ and Rₗ, identical or different, are chosen among (C₁-C₆)alkyl, (C₁-C₆)alkyl-OH, (C₁-C₆)alkenyl, (C₁-C₆)alkynyl, (C₃-C₇)cycloalkyl, aryl, heteroaryl, heterocycle, (C₁-C₆)alkyl(C₃-C₇)cycloalkyl, (C₁-C₆)alkylaryl, (C₁-C₆)alkylheteroaryl, and (C₁-C₆)alkylheterocycle;
R_{d} and Rᵢ are identical or different, and are a peptidic group attached via its carboxyl terminus;
Rₑ and Rⱼ are identical or different, and are a glycosidyl group;
R_{f} is chosen among hydrogen, —R_{g}, —COR_{g}, —CO₂R_{g}, —CONH₂, —CONR_{g}Rₕ, —CONHR_{g}, —OCH₂OC(O)R_{g}, and cleavable groups which after, for instance in vivo, lead either to R₆ or R₇═—OH when Y₂═O, to R₆ or R₇=—NH₂ when Y₂═NH, or to R₆ or R₇=—NHR₁ when Y₂═NR₁, or to R₆ or R₇=—SH when Y₂═S;
Rₙ is chosen among —CH₃, —OH, —NH₂, —NHCOCH₃, —SO₃H, —CO₂H, —CONH₂, —CO₂CH₃, —PO₃H₂, —NO₂, —B(OH)₂, —N₃, —CN, —C≡CH, and —SO₂NH₂;
and their hydrates, pharmaceutically acceptable salts and solvates, as a mixture of isomers in any proportions and also as pure isomer;
wherein the compounds are in the trans form of the diazenyl bond, or in a mixture of cis and trans forms of the diazenyl bond.

81. A compound for use according to claim 74, wherein the azoaryl compound is an azobenzene compound.

82. A method of studying the cytoskeleton and/or its associated processes wherein cells or a sample are treated with an azoaryl compound, at least partially in its trans isomeric form of the diazenyl bond, which is inactive as regards a tubulin polymerisation inhibitory effect, and where the compound is photoisomerised in vitro to an azoaryl compound in its cis isomeric form of the diazenyl bond by the application of light, with optional modification in vitro of one or more substituents either before or after this photoisomerisation, and where this cis form is active as regards a tubulin polymerisation inhibitory effect.

83. A method according to claim 82, wherein:
either, the azoaryl compound in its pure trans isomeric form of the diazenyl bond is the form of the compound used for treating the cells or the sample and where its cis form is active as regards a tubulin polymerisation inhibitory effect;
or, the azoaryl compound in a mixture of its cis and trans isomeric forms of the diazenyl bond is the form of the compound used for treating the cells or the sample and where its cis form is active as regards a tubulin polymerisation inhibitory effect.

84. A method according to claim 82, wherein the application of light is localized.

85. A method according to claim 82, wherein the conversion from the trans to the cis form of the diazenyl bond is followed by its conversion from the cis to the trans form by spontaneous thermal reversion or by application of light with a wavelength able to isomerise the compound from its cis form to its trans form of the diazenyl bond.

86. A method according to claim 85, wherein the isomerisation of the diazenyl bond from the cis to the trans form leads to an inactive form as regards a tubulin polymerisation inhibitory effect.

87. A method according to claim 82, wherein the compound for treating the cells or sample is selected among the compounds: 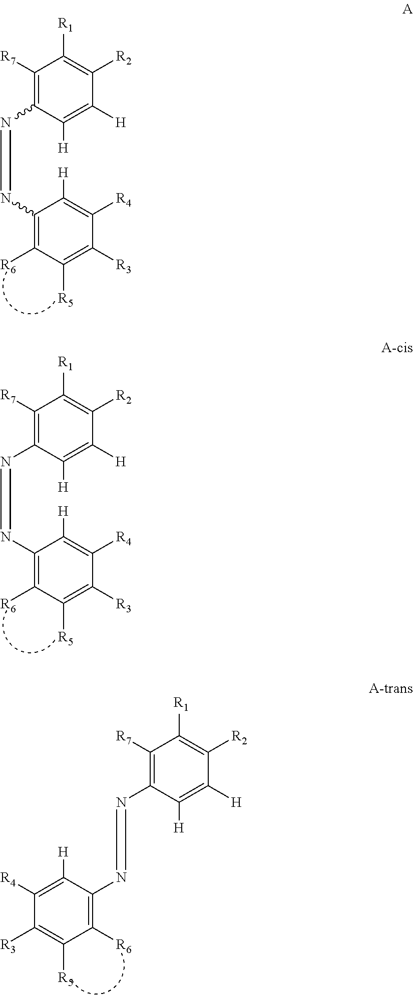
wherein:
the aryl ring bearing R₃ is denoted the “south ring”,
dotted lines indicate sites where a fused ring may be present;
R₂ is chosen among —OCH₃, —OCF₃, —F, —CH₃, —CF₃, —CH₂CH₃, —OCH₂CH₃, —SCH₃, —SCF₃, NHCH₃, —N(CH₃)₂ and —CN; and
R₁, R₆ and R₇ are defined as follows:
R₁ is chosen among hydrogen, —Y₁Rₐ, —S₂R_{b}, —NHR_{d}, —ORₑ, —OPO₃H₂, —NO₂, —B(OH)₂, —B(OR_{b})₂, —B(OR_{b}O), —N₃, —F, —Cl, —Br, —I, —CHO, —CO₂H, —CONH₂, —CN, —NC, —SO₃H, —CO₂R_{b}, —SO₂NH₂ and —R_{b};
R₇, and when R₆ is not linked to R₅, R₆ also, identical or different, are chosen among hydrogen, —Y₂R_{f}, —S₂R_{g}, —NHRᵢ, —ORⱼ, —OPO₃H₂, —NO₂, —B(OH)₂, —B(OR_{g})₂, —B(OR_{g}O), —N₃, —F, —Cl, —Br, —I, —CHO, —CO₂H, —CONH₂, —CN, —NC, —SO₃H, —CO₂R_{g}, —SO₂NH₂, —R_{g}, —CO₂NHR_{g}, —CO₂NR_{g}Rₕ, —N-piperazinyl, —N-morpholinyl, —N-pyrrolidinyl, —N— piperidinyl, and -linker-reporter units; and when R₆ is linked to R₅ then they are linked together forming a fused phenyl, 2-pyridinyl or 3-pyridinyl ring, the said phenyl, 2-pyridinyl or 3-pyridinyl being unsubstituted or substituted with one or several groups Rₙ, identical or different, such that the south ring is respectively a naphthalene, quinoline or isoquinoline;
considering that at least one of the substituents R₆, R₇ and R₁ is different from hydrogen; and
R₃ is chosen among —OCH₃, —OCF₃, —F, —CH₃, —CF₃, —CH₂CH₃, —OCH₂CH₃, —SCH₃, —SCF₃, —NHCH₃, —N(CH₃)₂ and —CN;
R₄ and, when R₆ is not linked to R₅, R₅ also, identical or different, are chosen among —OCH₃, —OCF₃, —F, —CH₃, —CF₃, —CH₂CH₃, —OCH₂CH₃, —SCH₃, —SCF₃, —NH₂, —NHCH₃, —N(CH₃)₂ and —CN;
Y₁═O, S, NH or NRₖ;
Y₂═O, S, NH or NRₗ;
Rₐ is chosen among hydrogen, —R_{b}, —COR_{b}, —CO₂R_{b}, —CONH₂, —CONR_{b}R_{c}, —CONHR_{b}, —CH₂OC(O)R_{b}, and cleavable groups which after cleavage, for instance in vivo, lead either to R₁═—OH when Y₁═O, or to R₁═—NH₂ when Y₁═NH, or to R₁═—NHRₖ when Y₁═NRₖ, or to R₁═—SH when Y₁═S;
R_{b}, R_{c}, R_{g}, Rₕ, Rₖ and Rₗ, identical or different, are chosen among (C₁-C₆)alkyl, (C₁-C₆)alkyl-OH, (C₁-C₆)alkenyl, (C₁-C₆)alkynyl, (C₃-C₇)cycloalkyl, aryl, heteroaryl, heterocycle, (C₁-C₆)alkyl(C₃-C₇)cycloalkyl, (C₁-C₆)alkylaryl, (C₁-C₆)alkylheteroaryl, and (C₁-C₆)alkylheterocycle;
R_{d} and Rᵢ are identical or different, and are a peptidic group attached via its carboxyl terminus;
Rₑ and Rⱼ are identical or different, and are a glycosidyl group;
R_{f} is chosen among hydrogen, —R_{g}, —COR_{g}, —CO₂R_{g}, —CONH₂, —CONR_{g}Rₕ, —CONHR_{g}, —OCH₂OC(O)R_{g}, and cleavable groups which after, for instance in vivo, lead either to R₆ or R₇═—OH when Y₂═O, to R₆ or R₇═—NH₂ when Y₂═NH, or to R₆ or R₇═—NHR₁ when Y₂═NR₁, or to R₆ or R₇═—SH when Y₂═S;
Rₙ is chosen among —CH₃, —OH, —NH₂, —NHCOCH₃, —SO₃H, —CO₂H, —CONH₂, —CO₂CH₃, —PO₃H₂, —NO₂, —B(OH)₂, —N₃, —CN, —C≡CH, and —SO₂NH₂;
and their hydrates, pharmaceutically acceptable salts and solvates, as a mixture of isomers in any proportions and also as pure isomer;
wherein the compounds are in the trans form of the diazenyl bond, or in a mixture of cis and trans forms of the diazenyl bond.

88. A method according to claim 82, wherein the azoaryl compound is an azobenzene compound.

89. A method according to claim 82, wherein the compound is 1-(4-methoxynaphthalen-1-yl)-2-(3,4,5-trimethoxyphenyl)diazene or 1-(4-methoxyphenyl)-2-(3,4,5-trimethoxyphenyl)diazene in its trans form or in a mixture of its trans and cis forms.
